SEQUENCE LISTING (31 sequences)

SEQ ID NO: 1 - protein, 7096 aa, Severe acute respiratory syndrome coronavirus 2 [sequence omitted]

SEQ ID NO: 2 - DNA, 29903 bp, Severe acute respiratory syndrome coronavirus 2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 3 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 4405 aa, Severe acute respiratory syndrome coronavirus 2 [sequence omitted]

SEQ ID NO: 5 - protein, 275 aa, Severe acute respiratory syndrome coronavirus 2
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 6 - protein, 75 aa, Severe acute respiratory syndrome coronavirus 2
MYSFVSEETGTLIVNSVLLFLAFVVFLLVTLAILTALRLCAYCCNIVNVSLVKPSFYVYSRVKNLNSSRVPDLLV

SEQ ID NO: 7 - protein, 222 aa, Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 8 - protein, 61 aa, Severe acute respiratory syndrome coronavirus 2
MFHLVDFQVTIAEILLIIMRTFKVSIWNLDYIINLIIKNLSKSLTENKYSQLDEEQPMEID

SEQ ID NO: 9 - protein, 121 aa, Severe acute respiratory syndrome coronavirus 2
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 10 - protein, 43 aa, Severe acute respiratory syndrome coronavirus 2
MIELSLIDFYLCFLAFLLFLVLIMLIIFWFSLELQDHNETCHA

SEQ ID NO: 11 - protein, 121 aa, Severe acute respiratory syndrome coronavirus 2
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 12 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 13 - protein, 38 aa, Severe acute respiratory syndrome coronavirus 2
MGYINVFAFPFTIYSLLLCRMNSRNYIAQVDVVNFNLT

SEQ ID NO: 14 - DNA, 8208 bp, Artificial Sequence
  Synthetic
ttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgccctttacacgtacttagtcgctgaagcatgcgaggaagcggaagagccagctggcttctatcctacgtcagccatcaggctaaggtcgggactagaagacaagctttaatacgactcactataggcaactcttctcggtccccacagactcagagagaacccaccatggcgccccgcagcgcccggcgacccctgctgctgctactgctgttgctgctgctcggcctcatgcattgtgcgtcagcagcaatgtttatggtgaaaaatggcaacgggaccgcgtgcataatggccaacttctctgctgccttctcagtgaactacgacaccaagagtggccctaagaacatgacccttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgtccagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagccggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagcggcggccgcatgtttgtgttcctggtgctgctgccactggtgtccagccagtgtgtgaacctgaccaccaggacccaacttcctcctgcctacaccaactccttcaccaggggagtctactaccctgacaaggtgttcaggtcctctgtgctgcacagcacccaggacctgttcctgccattcttcagcaatgtgacctggttccatgccatccatgtgtctggcaccaatggcaccaagaggtttgacaaccctgtgctgccattcaatgatggagtctactttgccagcacagagaagagcaacatcatcaggggctggatttttggcaccaccctggacagcaagacccagtccctgctgattgtgaacaatgccaccaatgtggtgattaaggtgtgtgagttccagttctgtaatgacccattcctgggagtctactaccacaagaacaacaagtcctggatggagtctgagttcagggtctactcctctgccaacaactgtacctttgaatatgtgagccaaccattcctgatggacttggagggcaagcagggcaacttcaagaacctgagggagtttgtgttcaagaacattgatggctacttcaagatttacagcaaacacacaccaatcaacctggtgagggacctgccacagggcttctctgccttggaaccactggtggacctgccaattggcatcaacatcaccaggttccagaccctgctggctctgcacaggtcctacctgacacctggagactcctcctctggctggacagcaggagcagcagcctactatgtgggctacctccaaccaaggaccttcctgctgaaatacaatgagaatggcaccatcacagatgctgtggactgtgccctggacccactgtctgagaccaagtgtaccctgaaatccttcacagtggagaagggcatctaccagaccagcaacttcagggtccaaccaacagagagcattgtgaggtttccaaacatcaccaacctgtgtccatttggagaggtgttcaatgccaccaggtttgcctctgtctatgcctggaacaggaagaggattagcaactgtgtggctgactactctgtgctctacaactctgcctccttcagcaccttcaagtgttatggagtgagcccaaccaaactgaatgacctgtgtttcaccaatgtctatgctgactcctttgtgattaggggagatgaggtgagacagattgcccctggacaaacaggcaagattgctgactacaactacaaactgcctgatgacttcacaggctgtgtgattgcctggaacagcaacaacctggacagcaaggtgggaggcaactacaactacctctacagactgttcaggaagagcaacctgaaaccatttgagagggacatcagcacagagatttaccaggctggcagcacaccatgtaatggagtggagggcttcaactgttactttccactccaatcctatggcttccaaccaaccaatggagtgggctaccaaccatacagggtggtggtgctgtcctttgaactgctccatgcccctgccacagtgtgtggaccaaagaagagcaccaacctggtgaagaacaagtgtgtgaacttcaacttcaatggactgacaggcacaggagtgctgacagagagcaacaagaagttcctgccattccaacagtttggcagggacattgctgacaccacagatgctgtgagggacccacagaccttggagattctggacatcacaccatgttcctttggaggagtgtctgtgattacacctggcaccaacaccagcaaccaggtggctgtgctctaccaggatgtgaactgtactgaggtgcctgtggctatccatgctgaccaacttacaccaacctggagggtctacagcacaggcagcaatgtgttccagaccagggctggctgtctgattggagcagagcatgtgaacaactcctatgagtgtgacatcccaattggagcaggcatctgtgcctcctaccagacccagaccaacagcccaaggagggcaaggtctgtggcaagccagagcatcattgcctacacaatgagtctgggagcagagaactctgtggcttacagcaacaacagcattgccatcccaaccaacttcaccatctctgtgaccacagagattctgcctgtgagtatgaccaagacctctgtggactgtacaatgtatatctgtggagacagcacagagtgtagcaacctgctgctccaatatggctccttctgtacccaacttaacagggctctgacaggcattgctgtggaacaggacaagaacacccaggaggtgtttgcccaggtgaagcagatttacaagacacctccaatcaaggactttggaggcttcaacttcagccagattctgcctgacccaagcaagccaagcaagaggtccttcattgaggacctgctgttcaacaaggtgaccctggctgatgctggcttcatcaagcaatatggagactgtctgggagacattgctgccagggacctgatttgtgcccagaagttcaatggactgacagtgctgcctccactgctgacagatgagatgattgcccaatacacctctgccctgctggctggcaccatcacctctggctggacctttggagcaggagcagccctccaaatcccatttgctatgcagatggcttacaggttcaatggcattggagtgacccagaatgtgctctatgagaaccagaaactgattgccaaccagttcaactctgccattggcaagattcaggactccctgtccagcacagcctctgccctgggcaaactccaagatgtggtgaaccagaatgcccaggctctgaacaccctggtgaagcaactttccagcaactttggagccatctcctctgtgctgaatgacatcctgagcagactggacaaggtggaggctgaggtccagattgacagactgattacaggcagactccaatccctccaaacctatgtgacccaacaacttatcagggctgctgagattagggcatctgccaacctggctgccaccaagatgagtgagtgtgtgctgggacaaagcaagagggtggacttctgtggcaagggctaccacctgatgagttttccacagtctgcccctcatggagtggtgttcctgcatgtgacctatgtgcctgcccaggagaagaacttcaccacagcccctgccatctgccatgatggcaaggctcactttccaagggagggagtgtttgtgagcaatggcacccactggtttgtgacccagaggaacttctatgaaccacagattatcaccacagacaacacctttgtgtctggcaactgtgatgtggtgattggcattgtgaacaacacagtctatgacccactccaacctgaactggactccttcaaggaggaactggacaaatacttcaagaaccacaccagccctgatgtggacctgggagacatctctggcatcaatgcctctgtggtgaacatccagaaggagattgacagactgaatgaggtggctaagaacctgaatgagtccctgattgacctccaagaactgggcaaatatgaacaatacatcaagtggccatggtacatctggctgggcttcattgctggactgattgccattgtgatggtgaccataatgctgtgttgtatgacctcctgttgttcctgtctgaaaggctgttgttcctgtggctcctgttgtaagtttgatgaggatgactctgaacctgtgctgaaaggagtgaaactgcactacacctgatctagaacgctgatccccatcgctgtgggtggtgccctggcggggctggtcctcatcgtcctcatcgcctacctcgtcggcaggaagaggagtcacgcaggctaccagactatctaaggatccttaagctgccttctgcggggcttgccttctggccatgcccttcttctctcccttgcacctgtacctcttggtctttgaataaagcctgagtaggaagaaggtaccgaattcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagagaccgtcttcactagtgagctccggattgccatccaagagctaagtctaagataggggcgcccatatgcgtcaatcgagttcgtacctaagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctt

SEQ ID NO: 15 - DNA, 7716 bp, Artificial Sequence
  Synthetic
tcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctaatacgactcactatagggagacaagcttgcatgcctgcaggtcgactctagaggatccgccgccaccatggcgccccgcagcgcccggcgacccctgctgctgctactgctgttgctgctgctcggcctcatgcattgtgcgtcagcagcaatgtttatggtgaaaaatggcaacgggaccgcgtgcataatggccaacttctctgctgccttctcagtgaactacgacaccaagagtggccctaagaacatgacccttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgtccagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagccggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagcggcggccgcatgtttgtgttcctggtgctgctgccactggtgtccagccagtgtgtgaacctgaccaccaggacccaacttcctcctgcctacaccaactccttcaccaggggagtctactaccctgacaaggtgttcaggtcctctgtgctgcacagcacccaggacctgttcctgccattcttcagcaatgtgacctggttccatgccatccatgtgtctggcaccaatggcaccaagaggtttgacaaccctgtgctgccattcaatgatggagtctactttgccagcacagagaagagcaacatcatcaggggctggatttttggcaccaccctggacagcaagacccagtccctgctgattgtgaacaatgccaccaatgtggtgattaaggtgtgtgagttccagttctgtaatgacccattcctgggagtctactaccacaagaacaacaagtcctggatggagtctgagttcagggtctactcctctgccaacaactgtacctttgaatatgtgagccaaccattcctgatggacttggagggcaagcagggcaacttcaagaacctgagggagtttgtgttcaagaacattgatggctacttcaagatttacagcaaacacacaccaatcaacctggtgagggacctgccacagggcttctctgccttggaaccactggtggacctgccaattggcatcaacatcaccaggttccagaccctgctggctctgcacaggtcctacctgacacctggagactcctcctctggctggacagcaggagcagcagcctactatgtgggctacctccaaccaaggaccttcctgctgaaatacaatgagaatggcaccatcacagatgctgtggactgtgccctggacccactgtctgagaccaagtgtaccctgaaatccttcacagtggagaagggcatctaccagaccagcaacttcagggtccaaccaacagagagcattgtgaggtttccaaacatcaccaacctgtgtccatttggagaggtgttcaatgccaccaggtttgcctctgtctatgcctggaacaggaagaggattagcaactgtgtggctgactactctgtgctctacaactctgcctccttcagcaccttcaagtgttatggagtgagcccaaccaaactgaatgacctgtgtttcaccaatgtctatgctgactcctttgtgattaggggagatgaggtgagacagattgcccctggacaaacaggcaagattgctgactacaactacaaactgcctgatgacttcacaggctgtgtgattgcctggaacagcaacaacctggacagcaaggtgggaggcaactacaactacctctacagactgttcaggaagagcaacctgaaaccatttgagagggacatcagcacagagatttaccaggctggcagcacaccatgtaatggagtggagggcttcaactgttactttccactccaatcctatggcttccaaccaaccaatggagtgggctaccaaccatacagggtggtggtgctgtcctttgaactgctccatgcccctgccacagtgtgtggaccaaagaagagcaccaacctggtgaagaacaagtgtgtgaacttcaacttcaatggactgacaggcacaggagtgctgacagagagcaacaagaagttcctgccattccaacagtttggcagggacattgctgacaccacagatgctgtgagggacccacagaccttggagattctggacatcacaccatgttcctttggaggagtgtctgtgattacacctggcaccaacaccagcaaccaggtggctgtgctctaccaggatgtgaactgtactgaggtgcctgtggctatccatgctgaccaacttacaccaacctggagggtctacagcacaggcagcaatgtgttccagaccagggctggctgtctgattggagcagagcatgtgaacaactcctatgagtgtgacatcccaattggagcaggcatctgtgcctcctaccagacccagaccaacagcccaaggagggcaaggtctgtggcaagccagagcatcattgcctacacaatgagtctgggagcagagaactctgtggcttacagcaacaacagcattgccatcccaaccaacttcaccatctctgtgaccacagagattctgcctgtgagtatgaccaagacctctgtggactgtacaatgtatatctgtggagacagcacagagtgtagcaacctgctgctccaatatggctccttctgtacccaacttaacagggctctgacaggcattgctgtggaacaggacaagaacacccaggaggtgtttgcccaggtgaagcagatttacaagacacctccaatcaaggactttggaggcttcaacttcagccagattctgcctgacccaagcaagccaagcaagaggtccttcattgaggacctgctgttcaacaaggtgaccctggctgatgctggcttcatcaagcaatatggagactgtctgggagacattgctgccagggacctgatttgtgcccagaagttcaatggactgacagtgctgcctccactgctgacagatgagatgattgcccaatacacctctgccctgctggctggcaccatcacctctggctggacctttggagcaggagcagccctccaaatcccatttgctatgcagatggcttacaggttcaatggcattggagtgacccagaatgtgctctatgagaaccagaaactgattgccaaccagttcaactctgccattggcaagattcaggactccctgtccagcacagcctctgccctgggcaaactccaagatgtggtgaaccagaatgcccaggctctgaacaccctggtgaagcaactttccagcaactttggagccatctcctctgtgctgaatgacatcctgagcagactggacaaggtggaggctgaggtccagattgacagactgattacaggcagactccaatccctccaaacctatgtgacccaacaacttatcagggctgctgagattagggcatctgccaacctggctgccaccaagatgagtgagtgtgtgctgggacaaagcaagagggtggacttctgtggcaagggctaccacctgatgagttttccacagtctgcccctcatggagtggtgttcctgcatgtgacctatgtgcctgcccaggagaagaacttcaccacagcccctgccatctgccatgatggcaaggctcactttccaagggagggagtgtttgtgagcaatggcacccactggtttgtgacccagaggaacttctatgaaccacagattatcaccacagacaacacctttgtgtctggcaactgtgatgtggtgattggcattgtgaacaacacagtctatgacccactccaacctgaactggactccttcaaggaggaactggacaaatacttcaagaaccacaccagccctgatgtggacctgggagacatctctggcatcaatgcctctgtggtgaacatccagaaggagattgacagactgaatgaggtggctaagaacctgaatgagtccctgattgacctccaagaactgggcaaatatgaacaatacatcaagtggccatggtacatctggctgggcttcattgctggactgattgccattgtgatggtgaccataatgctgtgttgtatgacctcctgttgttcctgtctgaaaggctgttgttcctgtggctcctgttgtaagtttgatgaggatgactctgaacctgtgctgaaaggagtgaaactgcactacacctctagaacgctgatccccatcgctgtgggtggtgccctggcggggctggtcctcatcgtcctcatcgcctacctcgtcggcaggaagaggagtcacgcaggctaccagactatctagtaagatctaattcttaattaaaaaaaaaaaaaaaaaaaaaaaaaaaaaaaaaaaaaaaaaaaaaaaaaaaaaaaaaaaactagtggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtc

SEQ ID NO: 16 - DNA, 6950 bp, Artificial Sequence
  Synthetic
ttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgccctttacacgtacttagtcgctgaagcatgcgaggaagcggaagagccagctggcttctatcctacgtcagccatcaggctaaggtcgggactagaagacaagctttaatacgactcactataggcaactcttctcggtccccacagactcagagagaacccaccatgtttgtgttcctggtgctgctgccactggtgtccagccagtgtgtgaacctgaccaccaggacccaacttcctcctgcctacaccaactccttcaccaggggagtctactaccctgacaaggtgttcaggtcctctgtgctgcacagcacccaggacctgttcctgccattcttcagcaatgtgacctggttccatgccatccatgtgtctggcaccaatggcaccaagaggtttgacaaccctgtgctgccattcaatgatggagtctactttgccagcacagagaagagcaacatcatcaggggctggatttttggcaccaccctggacagcaagacccagtccctgctgattgtgaacaatgccaccaatgtggtgattaaggtgtgtgagttccagttctgtaatgacccattcctgggagtctactaccacaagaacaacaagtcctggatggagtctgagttcagggtctactcctctgccaacaactgtacctttgaatatgtgagccaaccattcctgatggacttggagggcaagcagggcaacttcaagaacctgagggagtttgtgttcaagaacattgatggctacttcaagatttacagcaaacacacaccaatcaacctggtgagggacctgccacagggcttctctgccttggaaccactggtggacctgccaattggcatcaacatcaccaggttccagaccctgctggctctgcacaggtcctacctgacacctggagactcctcctctggctggacagcaggagcagcagcctactatgtgggctacctccaaccaaggaccttcctgctgaaatacaatgagaatggcaccatcacagatgctgtggactgtgccctggacccactgtctgagaccaagtgtaccctgaaatccttcacagtggagaagggcatctaccagaccagcaacttcagggtccaaccaacagagagcattgtgaggtttccaaacatcaccaacctgtgtccatttggagaggtgttcaatgccaccaggtttgcctctgtctatgcctggaacaggaagaggattagcaactgtgtggctgactactctgtgctctacaactctgcctccttcagcaccttcaagtgttatggagtgagcccaaccaaactgaatgacctgtgtttcaccaatgtctatgctgactcctttgtgattaggggagatgaggtgagacagattgcccctggacaaacaggcaagattgctgactacaactacaaactgcctgatgacttcacaggctgtgtgattgcctggaacagcaacaacctggacagcaaggtgggaggcaactacaactacctctacagactgttcaggaagagcaacctgaaaccatttgagagggacatcagcacagagatttaccaggctggcagcacaccatgtaatggagtggagggcttcaactgttactttccactccaatcctatggcttccaaccaaccaatggagtgggctaccaaccatacagggtggtggtgctgtcctttgaactgctccatgcccctgccacagtgtgtggaccaaagaagagcaccaacctggtgaagaacaagtgtgtgaacttcaacttcaatggactgacaggcacaggagtgctgacagagagcaacaagaagttcctgccattccaacagtttggcagggacattgctgacaccacagatgctgtgagggacccacagaccttggagattctggacatcacaccatgttcctttggaggagtgtctgtgattacacctggcaccaacaccagcaaccaggtggctgtgctctaccaggatgtgaactgtactgaggtgcctgtggctatccatgctgaccaacttacaccaacctggagggtctacagcacaggcagcaatgtgttccagaccagggctggctgtctgattggagcagagcatgtgaacaactcctatgagtgtgacatcccaattggagcaggcatctgtgcctcctaccagacccagaccaacagcccaaggagggcaaggtctgtggcaagccagagcatcattgcctacacaatgagtctgggagcagagaactctgtggcttacagcaacaacagcattgccatcccaaccaacttcaccatctctgtgaccacagagattctgcctgtgagtatgaccaagacctctgtggactgtacaatgtatatctgtggagacagcacagagtgtagcaacctgctgctccaatatggctccttctgtacccaacttaacagggctctgacaggcattgctgtggaacaggacaagaacacccaggaggtgtttgcccaggtgaagcagatttacaagacacctccaatcaaggactttggaggcttcaacttcagccagattctgcctgacccaagcaagccaagcaagaggtccttcattgaggacctgctgttcaacaaggtgaccctggctgatgctggcttcatcaagcaatatggagactgtctgggagacattgctgccagggacctgatttgtgcccagaagttcaatggactgacagtgctgcctccactgctgacagatgagatgattgcccaatacacctctgccctgctggctggcaccatcacctctggctggacctttggagcaggagcagccctccaaatcccatttgctatgcagatggcttacaggttcaatggcattggagtgacccagaatgtgctctatgagaaccagaaactgattgccaaccagttcaactctgccattggcaagattcaggactccctgtccagcacagcctctgccctgggcaaactccaagatgtggtgaaccagaatgcccaggctctgaacaccctggtgaagcaactttccagcaactttggagccatctcctctgtgctgaatgacatcctgagcagactggacaaggtggaggctgaggtccagattgacagactgattacaggcagactccaatccctccaaacctatgtgacccaacaacttatcagggctgctgagattagggcatctgccaacctggctgccaccaagatgagtgagtgtgtgctgggacaaagcaagagggtggacttctgtggcaagggctaccacctgatgagttttccacagtctgcccctcatggagtggtgttcctgcatgtgacctatgtgcctgcccaggagaagaacttcaccacagcccctgccatctgccatgatggcaaggctcactttccaagggagggagtgtttgtgagcaatggcacccactggtttgtgacccagaggaacttctatgaaccacagattatcaccacagacaacacctttgtgtctggcaactgtgatgtggtgattggcattgtgaacaacacagtctatgacccactccaacctgaactggactccttcaaggaggaactggacaaatacttcaagaaccacaccagccctgatgtggacctgggagacatctctggcatcaatgcctctgtggtgaacatccagaaggagattgacagactgaatgaggtggctaagaacctgaatgagtccctgattgacctccaagaactgggcaaatatgaacaatacatcaagtggccatggtacatctggctgggcttcattgctggactgattgccattgtgatggtgaccataatgctgtgttgtatgacctcctgttgttcctgtctgaaaggctgttgttcctgtggctcctgttgtaagtttgatgaggatgactctgaacctgtgctgaaaggagtgaaactgcactacacctgagcggccgcggatccttaagctgccttctgcggggcttgccttctggccatgcccttcttctctcccttgcacctgtacctcttggtctttgaataaagcctgagtaggaagaaggtaccgaattcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagagaccgtcttcactagtgagctccggattgccatccaagagctaagtctaagataggggcgcccatatgcgtcaatcgagttcgtacctaagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctt

SEQ ID NO: 17 - DNA, 4388 bp, Artificial Sequence
  Synthetic
ttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgccctttacacgtacttagtcgctgaagcatgcgaggaagcggaagagccagctggcttctatcctacgtcagccatcaggctaaggtcgggactagaagacaagctttaatacgactcactataggcaactcttctcggtccccacagactcagagagaacccaccatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaagcggccgcggatccttaagctgccttctgcggggcttgccttctggccatgcccttcttctctcccttgcacctgtacctcttggtctttgaataaagcctgagtaggaagaaggtaccgaattcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagagaccgtcttcactagtgagctccggattgccatccaagagctaagtctaagataggggcgcccatatgcgtcaatcgagttcgtacctaagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctt

SEQ ID NO: 18 - DNA, 3878 bp, Artificial Sequence
  Synthetic
gcttccggctcgtatgttgtgtggaattgtgagcggataacaatttcacacaggaaacagctatgaccatgattacgccaagctctaatacgactcactatagggagacaagcttgcggccgcatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaggatccccgggtaccgagctcgaattcttaattaaaaaaaaaaaaaaaaaaaaaaaaaaaaaaaaaaaaaaaaaaaaaaaaaaaaaaaaaaaaaactagtggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatcgaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactcgaaagcgggcagtgagcgcaacgcaattaatgtgagttagctcactcattaggcaccccaggctttacactttat

SEQ ID NO: 19 - DNA, 3797 bp, Artificial Sequence
  Synthetic
ttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgccctttacacgtacttagtcgctgaagcatgcgaggaagcggaagagccagctggcttctatcctacgtcagccatcaggctaaggtcgggactagaagacaagctttaatacgactcactataggcaactcttctcggtccccacagactcagagagaacccaccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagcggccgcggatccttaagctgccttctgcggggcttgccttctggccatgcccttcttctctcccttgcacctgtacctcttggtctttgaataaagcctgagtaggaagaaggtaccgaattcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagagaccgtcttcactagtgagctccggattgccatccaagagctaagtctaagataggggcgcccatatgcgtcaatcgagttcgtacctaagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctt

SEQ ID NO: 20 - DNA, 3287 bp, Artificial Sequence
  Synthetic
gcttccggctcgtatgttgtgtggaattgtgagcggataacaatttcacacaggaaacagctatgaccatgattacgccaagctctaatacgactcactatagggagacaagcttgcggccgcatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaaggatccccgggtaccgagctcgaattcttaattaaaaaaaaaaaaaaaaaaaaaaaaaaaaaaaaaaaaaaaaaaaaaaaaaaaaaaaaaaaaaactagtggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatcgaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactcgaaagcgggcagtgagcgcaacgcaattaatgtgagttagctcactcattaggcaccccaggctttacactttat

SEQ ID NO: 21 - DNA, 3356 bp, Artificial Sequence
  Synthetic
ttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgccctttacacgtacttagtcgctgaagcatgcgaggaagcggaagagccagctggcttctatcctacgtcagccatcaggctaaggtcgggactagaagacaagctttaatacgactcactataggcaactcttctcggtccccacagactcagagagaacccaccatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaagcggccgcggatccttaagctgccttctgcggggcttgccttctggccatgcccttcttctctcccttgcacctgtacctcttggtctttgaataaagcctgagtaggaagaaggtaccgaattcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagagaccgtcttcactagtgagctccggattgccatccaagagctaagtctaagataggggcgcccatatgcgtcaatcgagttcgtacctaagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctt

SEQ ID NO: 22 - DNA, 2846 bp, Artificial Sequence
  Synthetic
gcttccggctcgtatgttgtgtggaattgtgagcggataacaatttcacacaggaaacagctatgaccatgattacgccaagctctaatacgactcactatagggagacaagcttgcggccgcatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaggatccccgggtaccgagctcgaattcttaattaaaaaaaaaaaaaaaaaaaaaaaaaaaaaaaaaaaaaaaaaaaaaaaaaaaaaaaaaaaaaactagtggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatcgaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactcgaaagcgggcagtgagcgcaacgcaattaatgtgagttagctcactcattaggcaccccaggctttacactttat

SEQ ID NO: 23 - DNA, 5363 bp, Artificial Sequence
  Synthetic
ggcaacucuucucgguccccacagacucagagagaacccaccauggcgccccgcagcgcccggcgaccccugcugcugcuacugcuguugcugcugcucggccucaugcauugugcgucagcagcaauguuuauggugaaaaauggcaacgggaccgcgugcauaauggccaacuucucugcugccuucucagugaacuacgacaccaagaguggcccuaagaacaugacccuugaccugccaucagaugccacaguggugcucaaccgcagcuccuguggaaaagagaacacuucugaccccagucucgugauugcuuuuggaagaggacauacacucacucucaauuucacgagaaaugcaacacguuacagcguccagcucaugaguuuuguuuauaacuugucagacacacaccuuuuccccaaugcgagcuccaaagaaaucaagacuguggaaucuauaacugacaucagggcagauauagauaaaaaauacagauguguuaguggcacccagguccacaugaacaacgugaccguaacgcuccaugaugccaccauccaggcguaccuuuccaacagcagcuucagccggggagagacacgcugugaacaagacaggccuuccccaaccacagcgcccccugcgccacccagccccucgcccucacccgugcccaagagccccucuguggacaaguacaacgugagcggcaccaacgggaccugccugcuggccagcauggggcugcagcugaaccucaccuaugagaggaaggacaacacgacggugacaaggcuucucaacaucaaccccaacaagaccucggccagcgggagcugcggcgcccaccuggugacucuggagcugcacagcgagggcaccaccguccugcucuuccaguucgggaugaaugcaaguucuagccgguuuuuccuacaaggaauccaguugaauacaauucuuccugacgccagagacccugccuuuaaagcugccaacggcucccugcgagcgcugcaggccacagucggcaauuccuacaagugcaacgcggaggagcacguccgugucacgaaggcguuuucagucaauauauucaaaguguggguccaggcuuucaagguggaagguggccaguuuggcucuguggaggagugucugcuggacgagaacagcggcggccgcauguuuguguuccuggugcugcugccacugguguccagccagugugugaaccugaccaccaggacccaacuuccuccugccuacaccaacuccuucaccaggggagucuacuacccugacaagguguucagguccucugugcugcacagcacccaggaccuguuccugccauucuucagcaaugugaccugguuccaugccauccaugugucuggcaccaauggcaccaagagguuugacaacccugugcugccauucaaugauggagucuacuuugccagcacagagaagagcaacaucaucaggggcuggauuuuuggcaccacccuggacagcaagacccagucccugcugauugugaacaaugccaccaauguggugauuaaggugugugaguuccaguucuguaaugacccauuccugggagucuacuaccacaagaacaacaaguccuggauggagucugaguucagggucuacuccucugccaacaacuguaccuuugaauaugugagccaaccauuccugauggacuuggagggcaagcagggcaacuucaagaaccugagggaguuuguguucaagaacauugauggcuacuucaagauuuacagcaaacacacaccaaucaaccuggugagggaccugccacagggcuucucugccuuggaaccacugguggaccugccaauuggcaucaacaucaccagguuccagacccugcuggcucugcacagguccuaccugacaccuggagacuccuccucuggcuggacagcaggagcagcagccuacuaugugggcuaccuccaaccaaggaccuuccugcugaaauacaaugagaauggcaccaucacagaugcuguggacugugcccuggacccacugucugagaccaaguguacccugaaauccuucacaguggagaagggcaucuaccagaccagcaacuucaggguccaaccaacagagagcauugugagguuuccaaacaucaccaaccuguguccauuuggagagguguucaaugccaccagguuugccucugucuaugccuggaacaggaagaggauuagcaacuguguggcugacuacucugugcucuacaacucugccuccuucagcaccuucaaguguuauggagugagcccaaccaaacugaaugaccuguguuucaccaaugucuaugcugacuccuuugugauuaggggagaugaggugagacagauugccccuggacaaacaggcaagauugcugacuacaacuacaaacugccugaugacuucacaggcugugugauugccuggaacagcaacaaccuggacagcaaggugggaggcaacuacaacuaccucuacagacuguucaggaagagcaaccugaaaccauuugagagggacaucagcacagagauuuaccaggcuggcagcacaccauguaauggaguggagggcuucaacuguuacuuuccacuccaauccuauggcuuccaaccaaccaauggagugggcuaccaaccauacagggugguggugcuguccuuugaacugcuccaugccccugccacaguguguggaccaaagaagagcaccaaccuggugaagaacaagugugugaacuucaacuucaauggacugacaggcacaggagugcugacagagagcaacaagaaguuccugccauuccaacaguuuggcagggacauugcugacaccacagaugcugugagggacccacagaccuuggagauucuggacaucacaccauguuccuuuggaggagugucugugauuacaccuggcaccaacaccagcaaccagguggcugugcucuaccaggaugugaacuguacugaggugccuguggcuauccaugcugaccaacuuacaccaaccuggagggucuacagcacaggcagcaauguguuccagaccagggcuggcugucugauuggagcagagcaugugaacaacuccuaugagugugacaucccaauuggagcaggcaucugugccuccuaccagacccagaccaacagcccaaggagggcaaggucuguggcaagccagagcaucauugccuacacaaugagucugggagcagagaacucuguggcuuacagcaacaacagcauugccaucccaaccaacuucaccaucucugugaccacagagauucugccugugaguaugaccaagaccucuguggacuguacaauguauaucuguggagacagcacagaguguagcaaccugcugcuccaauauggcuccuucuguacccaacuuaacagggcucugacaggcauugcuguggaacaggacaagaacacccaggagguguuugcccaggugaagcagauuuacaagacaccuccaaucaaggacuuuggaggcuucaacuucagccagauucugccugacccaagcaagccaagcaagagguccuucauugaggaccugcuguucaacaaggugacccuggcugaugcuggcuucaucaagcaauauggagacugucugggagacauugcugccagggaccugauuugugcccagaaguucaauggacugacagugcugccuccacugcugacagaugagaugauugcccaauacaccucugcccugcuggcuggcaccaucaccucuggcuggaccuuuggagcaggagcagcccuccaaaucccauuugcuaugcagauggcuuacagguucaauggcauuggagugacccagaaugugcucuaugagaaccagaaacugauugccaaccaguucaacucugccauuggcaagauucaggacucccuguccagcacagccucugcccugggcaaacuccaagauguggugaaccagaaugcccaggcucugaacacccuggugaagcaacuuuccagcaacuuuggagccaucuccucugugcugaaugacauccugagcagacuggacaagguggaggcugagguccagauugacagacugauuacaggcagacuccaaucccuccaaaccuaugugacccaacaacuuaucagggcugcugagauuagggcaucugccaaccuggcugccaccaagaugagugagugugugcugggacaaagcaagaggguggacuucuguggcaagggcuaccaccugaugaguuuuccacagucugccccucauggagugguguuccugcaugugaccuaugugccugcccaggagaagaacuucaccacagccccugccaucugccaugauggcaaggcucacuuuccaagggagggaguguuugugagcaauggcacccacugguuugugacccagaggaacuucuaugaaccacagauuaucaccacagacaacaccuuugugucuggcaacugugauguggugauuggcauugugaacaacacagucuaugacccacuccaaccugaacuggacuccuucaaggaggaacuggacaaauacuucaagaaccacaccagcccugauguggaccugggagacaucucuggcaucaaugccucuguggugaacauccagaaggagauugacagacugaaugagguggcuaagaaccugaaugagucccugauugaccuccaagaacugggcaaauaugaacaauacaucaaguggccaugguacaucuggcugggcuucauugcuggacugauugccauugugauggugaccauaaugcuguguuguaugaccuccuguuguuccugucugaaaggcuguuguuccuguggcuccuguuguaaguuugaugaggaugacucugaaccugugcugaaaggagugaaacugcacuacaccugaucuagaacgcugauccccaucgcuguggguggugcccuggcggggcugguccucaucguccucaucgccuaccucgucggcaggaagaggagucacgcaggcuaccagacuaucuaaggauccuuaagcugccuucugcggggcuugccuucuggccaugcccuucuucucucccuugcaccuguaccucuuggucuuugaauaaagccugaguaggaagaagguaccgaauucaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 24 - DNA, 5215 bp, Artificial Sequence
  Synthetic
ggagacaagcuugcaugccugcaggucgacucuagaggauccgccgccaccauggcgccccgcagcgcccggcgaccccugcugcugcuacugcuguugcugcugcucggccucaugcauugugcgucagcagcaauguuuauggugaaaaauggcaacgggaccgcgugcauaauggccaacuucucugcugccuucucagugaacuacgacaccaagaguggcccuaagaacaugacccuugaccugccaucagaugccacaguggugcucaaccgcagcuccuguggaaaagagaacacuucugaccccagucucgugauugcuuuuggaagaggacauacacucacucucaauuucacgagaaaugcaacacguuacagcguccagcucaugaguuuuguuuauaacuugucagacacacaccuuuuccccaaugcgagcuccaaagaaaucaagacuguggaaucuauaacugacaucagggcagauauagauaaaaaauacagauguguuaguggcacccagguccacaugaacaacgugaccguaacgcuccaugaugccaccauccaggcguaccuuuccaacagcagcuucagccggggagagacacgcugugaacaagacaggccuuccccaaccacagcgcccccugcgccacccagccccucgcccucacccgugcccaagagccccucuguggacaaguacaacgugagcggcaccaacgggaccugccugcuggccagcauggggcugcagcugaaccucaccuaugagaggaaggacaacacgacggugacaaggcuucucaacaucaaccccaacaagaccucggccagcgggagcugcggcgcccaccuggugacucuggagcugcacagcgagggcaccaccguccugcucuuccaguucgggaugaaugcaaguucuagccgguuuuuccuacaaggaauccaguugaauacaauucuuccugacgccagagacccugccuuuaaagcugccaacggcucccugcgagcgcugcaggccacagucggcaauuccuacaagugcaacgcggaggagcacguccgugucacgaaggcguuuucagucaauauauucaaaguguggguccaggcuuucaagguggaagguggccaguuuggcucuguggaggagugucugcuggacgagaacagcggcggccgcauguuuguguuccuggugcugcugccacugguguccagccagugugugaaccugaccaccaggacccaacuuccuccugccuacaccaacuccuucaccaggggagucuacuacccugacaagguguucagguccucugugcugcacagcacccaggaccuguuccugccauucuucagcaaugugaccugguuccaugccauccaugugucuggcaccaauggcaccaagagguuugacaacccugugcugccauucaaugauggagucuacuuugccagcacagagaagagcaacaucaucaggggcuggauuuuuggcaccacccuggacagcaagacccagucccugcugauugugaacaaugccaccaauguggugauuaaggugugugaguuccaguucuguaaugacccauuccugggagucuacuaccacaagaacaacaaguccuggauggagucugaguucagggucuacuccucugccaacaacuguaccuuugaauaugugagccaaccauuccugauggacuuggagggcaagcagggcaacuucaagaaccugagggaguuuguguucaagaacauugauggcuacuucaagauuuacagcaaacacacaccaaucaaccuggugagggaccugccacagggcuucucugccuuggaaccacugguggaccugccaauuggcaucaacaucaccagguuccagacccugcuggcucugcacagguccuaccugacaccuggagacuccuccucuggcuggacagcaggagcagcagccuacuaugugggcuaccuccaaccaaggaccuuccugcugaaauacaaugagaauggcaccaucacagaugcuguggacugugcccuggacccacugucugagaccaaguguacccugaaauccuucacaguggagaagggcaucuaccagaccagcaacuucaggguccaaccaacagagagcauugugagguuuccaaacaucaccaaccuguguccauuuggagagguguucaaugccaccagguuugccucugucuaugccuggaacaggaagaggauuagcaacuguguggcugacuacucugugcucuacaacucugccuccuucagcaccuucaaguguuauggagugagcccaaccaaacugaaugaccuguguuucaccaaugucuaugcugacuccuuugugauuaggggagaugaggugagacagauugccccuggacaaacaggcaagauugcugacuacaacuacaaacugccugaugacuucacaggcugugugauugccuggaacagcaacaaccuggacagcaaggugggaggcaacuacaacuaccucuacagacuguucaggaagagcaaccugaaaccauuugagagggacaucagcacagagauuuaccaggcuggcagcacaccauguaauggaguggagggcuucaacuguuacuuuccacuccaauccuauggcuuccaaccaaccaauggagugggcuaccaaccauacagggugguggugcuguccuuugaacugcuccaugccccugccacaguguguggaccaaagaagagcaccaaccuggugaagaacaagugugugaacuucaacuucaauggacugacaggcacaggagugcugacagagagcaacaagaaguuccugccauuccaacaguuuggcagggacauugcugacaccacagaugcugugagggacccacagaccuuggagauucuggacaucacaccauguuccuuuggaggagugucugugauuacaccuggcaccaacaccagcaaccagguggcugugcucuaccaggaugugaacuguacugaggugccuguggcuauccaugcugaccaacuuacaccaaccuggagggucuacagcacaggcagcaauguguuccagaccagggcuggcugucugauuggagcagagcaugugaacaacuccuaugagugugacaucccaauuggagcaggcaucugugccuccuaccagacccagaccaacagcccaaggagggcaaggucuguggcaagccagagcaucauugccuacacaaugagucugggagcagagaacucuguggcuuacagcaacaacagcauugccaucccaaccaacuucaccaucucugugaccacagagauucugccugugaguaugaccaagaccucuguggacuguacaauguauaucuguggagacagcacagaguguagcaaccugcugcuccaauauggcuccuucuguacccaacuuaacagggcucugacaggcauugcuguggaacaggacaagaacacccaggagguguuugcccaggugaagcagauuuacaagacaccuccaaucaaggacuuuggaggcuucaacuucagccagauucugccugacccaagcaagccaagcaagagguccuucauugaggaccugcuguucaacaaggugacccuggcugaugcuggcuucaucaagcaauauggagacugucugggagacauugcugccagggaccugauuugugcccagaaguucaauggacugacagugcugccuccacugcugacagaugagaugauugcccaauacaccucugcccugcuggcuggcaccaucaccucuggcuggaccuuuggagcaggagcagcccuccaaaucccauuugcuaugcagauggcuuacagguucaauggcauuggagugacccagaaugugcucuaugagaaccagaaacugauugccaaccaguucaacucugccauuggcaagauucaggacucccuguccagcacagccucugcccugggcaaacuccaagauguggugaaccagaaugcccaggcucugaacacccuggugaagcaacuuuccagcaacuuuggagccaucuccucugugcugaaugacauccugagcagacuggacaagguggaggcugagguccagauugacagacugauuacaggcagacuccaaucccuccaaaccuaugugacccaacaacuuaucagggcugcugagauuagggcaucugccaaccuggcugccaccaagaugagugagugugugcugggacaaagcaagaggguggacuucuguggcaagggcuaccaccugaugaguuuuccacagucugccccucauggagugguguuccugcaugugaccuaugugccugcccaggagaagaacuucaccacagccccugccaucugccaugauggcaaggcucacuuuccaagggagggaguguuugugagcaauggcacccacugguuugugacccagaggaacuucuaugaaccacagauuaucaccacagacaacaccuuugugucuggcaacugugauguggugauuggcauugugaacaacacagucuaugacccacuccaaccugaacuggacuccuucaaggaggaacuggacaaauacuucaagaaccacaccagcccugauguggaccugggagacaucucuggcaucaaugccucuguggugaacauccagaaggagauugacagacugaaugagguggcuaagaaccugaaugagucccugauugaccuccaagaacugggcaaauaugaacaauacaucaaguggccaugguacaucuggcugggcuucauugcuggacugauugccauugugauggugaccauaaugcuguguuguaugaccuccuguuguuccugucugaaaggcuguuguuccuguggcuccuguuguaaguuugaugaggaugacucugaaccugugcugaaaggagugaaacugcacuacaccucuagaacgcugauccccaucgcuguggguggugcccuggcggggcugguccucaucguccucaucgccuaccucgucggcaggaagaggagucacgcaggcuaccagacuaucuaguaagaucuaauucuuaauuaaaaaaaaaaaaaaaaaaaaaaaaaaaaaaaaaaaaaaaaaaaaaaaaaaaaaaaaaaaa

SEQ ID NO: 25 - DNA, 4105 bp, Artificial Sequence
  Synthetic
ggcaacucuucucgguccccacagacucagagagaacccaccauguuuguguuccuggugcugcugccacugguguccagccagugugugaaccugaccaccaggacccaacuuccuccugccuacaccaacuccuucaccaggggagucuacuacccugacaagguguucagguccucugugcugcacagcacccaggaccuguuccugccauucuucagcaaugugaccugguuccaugccauccaugugucuggcaccaauggcaccaagagguuugacaacccugugcugccauucaaugauggagucuacuuugccagcacagagaagagcaacaucaucaggggcuggauuuuuggcaccacccuggacagcaagacccagucccugcugauugugaacaaugccaccaauguggugauuaaggugugugaguuccaguucuguaaugacccauuccugggagucuacuaccacaagaacaacaaguccuggauggagucugaguucagggucuacuccucugccaacaacuguaccuuugaauaugugagccaaccauuccugauggacuuggagggcaagcagggcaacuucaagaaccugagggaguuuguguucaagaacauugauggcuacuucaagauuuacagcaaacacacaccaaucaaccuggugagggaccugccacagggcuucucugccuuggaaccacugguggaccugccaauuggcaucaacaucaccagguuccagacccugcuggcucugcacagguccuaccugacaccuggagacuccuccucuggcuggacagcaggagcagcagccuacuaugugggcuaccuccaaccaaggaccuuccugcugaaauacaaugagaauggcaccaucacagaugcuguggacugugcccuggacccacugucugagaccaaguguacccugaaauccuucacaguggagaagggcaucuaccagaccagcaacuucaggguccaaccaacagagagcauugugagguuuccaaacaucaccaaccuguguccauuuggagagguguucaaugccaccagguuugccucugucuaugccuggaacaggaagaggauuagcaacuguguggcugacuacucugugcucuacaacucugccuccuucagcaccuucaaguguuauggagugagcccaaccaaacugaaugaccuguguuucaccaaugucuaugcugacuccuuugugauuaggggagaugaggugagacagauugccccuggacaaacaggcaagauugcugacuacaacuacaaacugccugaugacuucacaggcugugugauugccuggaacagcaacaaccuggacagcaaggugggaggcaacuacaacuaccucuacagacuguucaggaagagcaaccugaaaccauuugagagggacaucagcacagagauuuaccaggcuggcagcacaccauguaauggaguggagggcuucaacuguuacuuuccacuccaauccuauggcuuccaaccaaccaauggagugggcuaccaaccauacagggugguggugcuguccuuugaacugcuccaugccccugccacaguguguggaccaaagaagagcaccaaccuggugaagaacaagugugugaacuucaacuucaauggacugacaggcacaggagugcugacagagagcaacaagaaguuccugccauuccaacaguuuggcagggacauugcugacaccacagaugcugugagggacccacagaccuuggagauucuggacaucacaccauguuccuuuggaggagugucugugauuacaccuggcaccaacaccagcaaccagguggcugugcucuaccaggaugugaacuguacugaggugccuguggcuauccaugcugaccaacuuacaccaaccuggagggucuacagcacaggcagcaauguguuccagaccagggcuggcugucugauuggagcagagcaugugaacaacuccuaugagugugacaucccaauuggagcaggcaucugugccuccuaccagacccagaccaacagcccaaggagggcaaggucuguggcaagccagagcaucauugccuacacaaugagucugggagcagagaacucuguggcuuacagcaacaacagcauugccaucccaaccaacuucaccaucucugugaccacagagauucugccugugaguaugaccaagaccucuguggacuguacaauguauaucuguggagacagcacagaguguagcaaccugcugcuccaauauggcuccuucuguacccaacuuaacagggcucugacaggcauugcuguggaacaggacaagaacacccaggagguguuugcccaggugaagcagauuuacaagacaccuccaaucaaggacuuuggaggcuucaacuucagccagauucugccugacccaagcaagccaagcaagagguccuucauugaggaccugcuguucaacaaggugacccuggcugaugcuggcuucaucaagcaauauggagacugucugggagacauugcugccagggaccugauuugugcccagaaguucaauggacugacagugcugccuccacugcugacagaugagaugauugcccaauacaccucugcccugcuggcuggcaccaucaccucuggcuggaccuuuggagcaggagcagcccuccaaaucccauuugcuaugcagauggcuuacagguucaauggcauuggagugacccagaaugugcucuaugagaaccagaaacugauugccaaccaguucaacucugccauuggcaagauucaggacucccuguccagcacagccucugcccugggcaaacuccaagauguggugaaccagaaugcccaggcucugaacacccuggugaagcaacuuuccagcaacuuuggagccaucuccucugugcugaaugacauccugagcagacuggacaagguggaggcugagguccagauugacagacugauuacaggcagacuccaaucccuccaaaccuaugugacccaacaacuuaucagggcugcugagauuagggcaucugccaaccuggcugccaccaagaugagugagugugugcugggacaaagcaagaggguggacuucuguggcaagggcuaccaccugaugaguuuuccacagucugccccucauggagugguguuccugcaugugaccuaugugccugcccaggagaagaacuucaccacagccccugccaucugccaugauggcaaggcucacuuuccaagggagggaguguuugugagcaauggcacccacugguuugugacccagaggaacuucuaugaaccacagauuaucaccacagacaacaccuuugugucuggcaacugugauguggugauuggcauugugaacaacacagucuaugacccacuccaaccugaacuggacuccuucaaggaggaacuggacaaauacuucaagaaccacaccagcccugauguggaccugggagacaucucuggcaucaaugccucuguggugaacauccagaaggagauugacagacugaaugagguggcuaagaaccugaaugagucccugauugaccuccaagaacugggcaaauaugaacaauacaucaaguggccaugguacaucuggcugggcuucauugcuggacugauugccauugugauggugaccauaaugcuguguuguaugaccuccuguuguuccugucugaaaggcuguuguuccuguggcuccuguuguaaguuugaugaggaugacucugaaccugugcugaaaggagugaaacugcacuacaccugagcggccgcggauccuuaagcugccuucugcggggcuugccuucuggccaugcccuucuucucucccuugcaccuguaccucuuggucuuugaauaaagccugaguaggaagaagguaccgaauucaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 26 - DNA, 1543 bp, Artificial Sequence
  Synthetic
ggcaacucuucucgguccccacagacucagagagaacccaccaugucugauaauggaccccaaaaucagcgaaaugcaccccgcauuacguuugguggacccucagauucaacuggcaguaaccagaauggagaacgcaguggggcgcgaucaaaacaacgucggccccaagguuuacccaauaauacugcgucuugguucaccgcucucacucaacauggcaaggaagaccuuaaauucccucgaggacaaggcguuccaauuaacaccaauagcaguccagaugaccaaauuggcuacuaccgaagagcuaccagacgaauucgugguggugacgguaaaaugaaagaucucaguccaagaugguauuucuacuaccuaggaacugggccagaagcuggacuucccuauggugcuaacaaagacggcaucauauggguugcaacugagggagccuugaauacaccaaaagaucacauuggcacccgcaauccugcuaacaaugcugcaaucgugcuacaacuuccucaaggaacaacauugccaaaaggcuucuacgcagaagggagcagaggcggcagucaagccucuucucguuccucaucacguagucgcaacaguucaagaaauucaacuccaggcagcaguaggggaacuucuccugcuagaauggcuggcaauggcggugaugcugcucuugcuuugcugcugcuugacagauugaaccagcuugagagcaaaaugucugguaaaggccaacaacaacaaggccaaacugucacuaagaaaucugcugcugaggcuucuaagaagccucggcaaaaacguacugccacuaaagcauacaauguaacacaagcuuucggcagacgugguccagaacaaacccaaggaaauuuuggggaccaggaacuaaucagacaaggaacugauuacaaacauuggccgcaaauugcacaauuugcccccagcgcuucagcguucuucggaaugucgcgcauuggcauggaagucacaccuucgggaacgugguugaccuacacaggugccaucaaauuggaugacaaagauccaaauuucaaagaucaagucauuuugcugaauaagcauauugacgcauacaaaacauucccaccaacagagccuaaaaaggacaaaaagaagaaggcugaugaaacucaagccuuaccgcagagacagaagaaacagcaaacugugacucuucuuccugcugcagauuuggaugauuucuccaaacaauugcaacaauccaugagcagugcugacucaacucaggccuaagcggccgcggauccuuaagcugccuucugcggggcuugccuucuggccaugcccuucuucucucccuugcaccuguaccucuuggucuuugaauaaagccugaguaggaagaagguaccgaauucaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 27 - DNA, 1375 bp, Artificial Sequence
  Synthetic
ggagacaagcuugcggccgcaugucugauaauggaccccaaaaucagcgaaaugcaccccgcauuacguuugguggacccucagauucaacuggcaguaaccagaauggagaacgcaguggggcgcgaucaaaacaacgucggccccaagguuuacccaauaauacugcgucuugguucaccgcucucacucaacauggcaaggaagaccuuaaauucccucgaggacaaggcguuccaauuaacaccaauagcaguccagaugaccaaauuggcuacuaccgaagagcuaccagacgaauucgugguggugacgguaaaaugaaagaucucaguccaagaugguauuucuacuaccuaggaacugggccagaagcuggacuucccuauggugcuaacaaagacggcaucauauggguugcaacugagggagccuugaauacaccaaaagaucacauuggcacccgcaauccugcuaacaaugcugcaaucgugcuacaacuuccucaaggaacaacauugccaaaaggcuucuacgcagaagggagcagaggcggcagucaagccucuucucguuccucaucacguagucgcaacaguucaagaaauucaacuccaggcagcaguaggggaacuucuccugcuagaauggcuggcaauggcggugaugcugcucuugcuuugcugcugcuugacagauugaaccagcuugagagcaaaaugucugguaaaggccaacaacaacaaggccaaacugucacuaagaaaucugcugcugaggcuucuaagaagccucggcaaaaacguacugccacuaaagcauacaauguaacacaagcuuucggcagacgugguccagaacaaacccaaggaaauuuuggggaccaggaacuaaucagacaaggaacugauuacaaacauuggccgcaaauugcacaauuugcccccagcgcuucagcguucuucggaaugucgcgcauuggcauggaagucacaccuucgggaacgugguugaccuacacaggugccaucaaauuggaugacaaagauccaaauuucaaagaucaagucauuuugcugaauaagcauauugacgcauacaaaacauucccaccaacagagccuaaaaaggacaaaaagaagaaggcugaugaaacucaagccuuaccgcagagacagaagaaacagcaaacugugacucuucuuccugcugcagauuuggaugauuucuccaaacaauugcaacaauccaugagcagugcugacucaacucaggccuaaggauccccggguaccgagcucgaauucuuaauuaaaaaaaaaaaaaaaaaaaaaaaaaaaaaaaaaaaaaaaaaaaaaaaaaaaaaaaaaaaaaa

SEQ ID NO: 28 - DNA, 952 bp, Artificial Sequence
  Synthetic
ggcaacucuucucgguccccacagacucagagagaacccaccauggcagauuccaacgguacuauuaccguugaagagcuuaaaaagcuccuugaacaauggaaccuaguaauagguuuccuauuccuuacauggauuugucuucuacaauuugccuaugccaacaggaauagguuuuuguauauaauuaaguuaauuuuccucuggcuguuauggccaguaacuuuagcuuguuuugugcuugcugcuguuuacagaauaaauuggaucaccgguggaauugcuaucgcaauggcuugucuuguaggcuugauguggcucagcuacuucauugcuucuuucagacuguuugcgcguacgcguuccauguggucauucaauccagaaacuaacauucuucucaacgugccacuccauggcacuauucugaccagaccgcuucuagaaagugaacucguaaucggagcugugauccuucguggacaucuucguauugcuggacaccaucuaggacgcugugacaucaaggaccugccuaaagaaaucacuguugcuacaucacgaacgcuuucuuauuacaaauugggagcuucgcagcguguagcaggugacucagguuuugcugcauacagucgcuacaggauuggcaacuauaaauuaaacacagaccauuccaguagcagugacaauauugcuuugcuuguacaguaagcggccgcggauccuuaagcugccuucugcggggcuugccuucuggccaugcccuucuucucucccuugcaccuguaccucuuggucuuugaauaaagccugaguaggaagaagguaccgaauucaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 29 - DNA, 784 bp, Artificial Sequence
  Synthetic
ggagacaagcuugcggccgcauggcagauuccaacgguacuauuaccguugaagagcuuaaaaagcuccuugaacaauggaaccuaguaauagguuuccuauuccuuacauggauuugucuucuacaauuugccuaugccaacaggaauagguuuuuguauauaauuaaguuaauuuuccucuggcuguuauggccaguaacuuuagcuuguuuugugcuugcugcuguuuacagaauaaauuggaucaccgguggaauugcuaucgcaauggcuugucuuguaggcuugauguggcucagcuacuucauugcuucuuucagacuguuugcgcguacgcguuccauguggucauucaauccagaaacuaacauucuucucaacgugccacuccauggcacuauucugaccagaccgcuucuagaaagugaacucguaaucggagcugugauccuucguggacaucuucguauugcuggacaccaucuaggacgcugugacaucaaggaccugccuaaagaaaucacuguugcuacaucacgaacgcuuucuuauuacaaauugggagcuucgcagcguguagcaggugacucagguuuugcugcauacagucgcuacaggauuggcaacuauaaauuaaacacagaccauuccaguagcagugacaauauugcuuugcuuguacaguaaggauccccggguaccgagcucgaauucuuaauuaaaaaaaaaaaaaaaaaaaaaaaaaaaaaaaaaaaaaaaaaaaaaaaaaaaaaaaaaaaaaa

SEQ ID NO: 30 - DNA, 511 bp, Artificial Sequence
  Synthetic
ggcaacucuucucgguccccacagacucagagagaacccaccauguacucauucguuucggaagagacagguacguuaauaguuaauagcguacuucuuuuucuugcuuucgugguauucuugcuaguuacacuagccauccuuacugcgcuucgauugugugcguacugcugcaauauuguuaacgugagucuuguaaaaccuucuuuuuacguuuacucucguguuaaaaaucugaauucuucuagaguuccugaucuucuggucuaagcggccgcggauccuuaagcugccuucugcggggcuugccuucuggccaugcccuucuucucucccuugcaccuguaccucuuggucuuugaauaaagccugaguaggaagaagguaccgaauucaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 31 - DNA, 343 bp, Artificial Sequence
  Synthetic
ggagacaagcuugcggccgcauguacucauucguuucggaagagacagguacguuaauaguuaauagcguacuucuuuuucuugcuuucgugguauucuugcuaguuacacuagccauccuuacugcgcuucgauugugugcguacugcugcaauauuguuaacgugagucuuguaaaaccuucuuuuuacguuuacucucguguuaaaaaucugaauucuucuagaguuccugaucuucuggucuaaggauccccggguaccgagcucgaauucuuaauuaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2023/0226169 A1
(43) Pub. Date: Jul. 20, 2023

(54) MULTILAMELLAR RNA NANOPARTICLE VACCINE AGAINST SARS-COV-2

(71) Applicant: UNIVERSITY OF FLORIDA RESEARCH FOUNDATION INCORPORATED, GAINESVILLE, FL (US)

(72) Inventors: Elias Sayour, Newberry, FL (US); Hector Ruben Mendez-Gomez, Gainesville, FL (US); Paul Antonio Castillo Caro, Gainesville, FL (US); Noah Thomas Jones, Gainesville, FL (US); Duane A. Mitchell, Gainesville, FL (US)

(21) Appl. No.: 17/914,894

(22) PCT Filed: Mar. 31, 2021
(86) PCT No.: PCT/US2021/025222

§ 371 Date: Sep. 27, 2022

Related U.S. Application Data
(60) Provisional application No. 63/128,600, filed on Dec. 21, 2020.
(60) Provisional application No. 63/022,999, filed on May 11, 2020.
(60) Provisional application No. 63/003,766, filed on Apr. 1, 2020.

(51) Int. Cl. A61K 39/215; A61P 31/14

(52) U.S. Cl. CPC A61K 39/215; A61P 31/14; A61K 2039/55555

(57) ABSTRACT

The present disclosure provides a nanoparticle comprising a positively-charged surface and an interior comprising (i) a core and (ii) at least two nucleic acid layers, wherein each nucleic acid layer is positioned between a cationic lipid bilayer, wherein the nanoparticle comprises RNA molecules encoding a SARS-CoV-2 protein. Methods of making such nanoparticles are further provided herein. Additionally, related cells, populations of cells, pharmaceutical compositions comprising the presently disclosed nanoparticles are provided. Methods of increasing an immune response against a tumor in a subject, methods of delivering RNA molecules to an intra-tumoral microenvironment, lymph node, and/or a reticuloendothelial organ in a subject, and methods of treating a subject with a disease are furthermore provided.

### CROSS-REFERENCE TO RELATED APPLICATIONS

This application claims the benefit of priority to U.S. Provisional Pat. Application No. 63/003,766, filed on Apr. 1, 2020; U.S. Provisional Pat. Application No. 63/022,999, filed on May 11, 2020; and U.S. Provisional Pat. Application No. 63/128,600, filed on Dec. 21, 2020, the disclosures of which are hereby incorporated by reference in their entireties.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 251,6660 byte ASCII (Text) file named “55544_Seqlisting.txt”; created on Mar. 31, 2021.

### BACKGROUND

The Severe Acute Respiratory Syndrome Coronavirus 2 (SARS-CoV-2) is a novel virus belonging to the Coronaviridae family of enveloped viruses. SARS-CoV-2 is a positive-sense single-stranded RNA virus with genetic similarity to bat coronaviruses and was first isolated in January 2020 from patients in Wuhan, China (Hui et al., International J Infectious Diseases 91: 264-266 (2020)). To date, over 750,000 people have been infected by SARS-CoV-2 and over 35,000 people have died from Coronavirus Disease 2018 (COVID-19), the disease caused by SARS-CoV-2 infection. To reduce the number of SARS CoV-2-related deaths, a vaccine to safely induce immunity against SARS-CoV-2 is needed.

Lipid nanoparticles comprising mRNA as vaccines have been studied. See, e.g., Reichmuth et al., Ther Deliv 7(5): 319-334 (2016). RNA vaccines have several advantages over traditional modalities. RNA has potent effects on both the innate and adaptive immune system. RNA can act as a toll-like receptor (TLR) agonist for receptors 3, 7, and 8 inducing potent TLR dependent innate immunity. RNA can also stimulate intracellular pathogen recognition receptors (i.e., melanoma differentiation antigen 5 (MDA-5) and retinoic acid inducible gene I (RIG-I)) and culminates in activating both helper-CD4 and cytotoxic CD8 T cell responses. Unlike DNA vaccines mired by having to cross both cellular and nuclear membranes, RNA only requires access to the cytoplasm and carries a significant safety advantage since it cannot be integrated into the host-genome. Unlike many peptide vaccines, which have only been developed for specific HLA haplotypes (i.e., HLA-A2), RNA bypasses MHC class restriction and can be leveraged for the population at large.

There is a need for materials and methods to safely and effectively induce immunity against SARS-CoV-2.

### SUMMARY

Nanoparticles comprising a positively-charged surface and an interior comprising (i) a core and (ii) at least two nucleic acid layers, wherein each nucleic acid layer is positioned between a cationic lipid bilayer, mediate peripheral and intratumoral activation of dendritic cells (DCs). These multilamellar RNA NPs (ML RNA NPs) also demonstrated superior efficacy of multilamellar tumor specific RNA-NPs, relative to anionic LPX and RNA LPX, and demonstrated the ability to systemically activate DCs, induce antigen specific immunity, and elicit anti-tumor efficacy in vivo. The administration of multilamellar RNA NPs to diseased animals (e.g., tumor bearing mice) also led to increased survival of these animals. Without being bound to a particular theory, the multilamellar RNA nanoparticles of the present disclosure are capable of safely and effectively inducing immunity against SARS-CoV-2.

The present disclosure provides a nanoparticle comprising a positively-charged surface and an interior comprising (i) a core and (ii) at least two nucleic acid layers, wherein each nucleic acid layer is positioned between a cationic lipid bilayer, wherein the nanoparticle comprises RNA molecules encoding a SARS-CoV-2 protein, optionally, a SARS-CoV-2 Spike (S) protein. In exemplary embodiments, the nanoparticle comprises at least three nucleic acid layers, each of which is positioned between a cationic lipid bilayer. In exemplary aspects, the nanoparticle comprises at least four or five or more nucleic acid layers, each of which is positioned between a cationic lipid bilayer. In various aspects, the outermost layer of the nanoparticle comprises a cationic lipid bilayer. In various instances, the surface comprises a plurality of hydrophilic moieties of the cationic lipid of the cationic lipid bilayer. In exemplary aspects, the core comprises a cationic lipid bilayer. Optionally, the core comprises less than about 0.5 wt% nucleic acid. The diameter of the nanoparticle, in various aspects, is about 50 nm to about 250 nm in diameter, optionally, about 70 nm to about 200 nm in diameter. In exemplary instances, the nanoparticle is characterized by a zeta potential of about +40 mV to about +60 mV, optionally, about +45 mV to about +55 mV. The nanoparticle in various instances, has a zeta potential of about 50 mV. In some aspects, the nucleic acid molecules are present at a nucleic acid molecule:cationic lipid ratio of about 1 to about 5 to about 1 to about 25, such as about 1 to about 5 to about 1 to about 20, optionally, about 1 to about 15, about 1 to about 10, or about 1 to about 7.5. In various aspects, the nucleic acid molecules are RNA molecules, optionally, messenger RNA (mRNA).

In cancer-related aspects of the technology described herein, the mRNA is in vitro transcribed mRNA wherein the in vitro transcription template is cDNA made from RNA extracted from a sample.

The present disclosure also provides a method of making a nanoparticle comprising a positively-charged surface and an interior comprising (i) a core and (ii) at least two nucleic acid layers, wherein each nucleic acid layer is positioned between a cationic lipid bilayer, wherein the nanoparticle comprises RNA molecules encoding a SARS-CoV-2 protein, optionally, a SARS-CoV-2 Spike (S) protein, said method comprising: (A) mixing nucleic acid molecules and liposomes at a RNA: liposome ratio of 1 to about 5 to about 1 to about 25, such as about 1 to about 5 to about 1 to about 20, optionally, about 1 to about 15, about 1 to about 10, or about 1 to about 7.5, to obtain a RNA-coated liposomes, wherein the liposomes are made by a process of making liposomes comprising drying a lipid mixture comprising a cationic lipid and an organic solvent by evaporating the organic solvent under a vacuum; and (B) mixing the RNA-coated liposomes with a surplus amount of liposomes. In exemplary aspects, the lipid mixture comprises the cationic lipid and the organic solvent at a ratio of about 40 mg cationic lipid per mL organic solvent to about 60 mg cationic lipid per mL organic solvent, optionally, at a ratio of about 50 mg cationic lipid per mL organic solvent. In various instances, the process of making liposomes further comprises rehydrating the lipid mixture with a rehydration solution to form a rehydrated lipid mixture and then agitating, resting, and sizing the rehydrated lipid mixture. Optionally, sizing the rehydrated lipid mixture comprises sonicating, extruding and/or filtering the rehydrated lipid mixture. In exemplary aspects, the RNA molecules are mRNA encoding SARS-CoV-2 protein, optionally mRNA encoding the SARS-CoV-2 S protein.

Further provided herein are nanoparticles made by the presently disclosed method of making a nanoparticle. Additionally provided herein is a cell comprising a nanoparticle of the present disclosure. Optionally, the cell is an antigen presenting cell (APC), e.g., a dendritic cell (DC). The present disclosure also provides a population of cells, wherein at least 50% of the population are cells according to the present disclosure. Pharmaceutical compositions comprising such cells comprising a nanoparticle of the present disclosure, or a population of such cells, are provided herein. The cells, or population of cells, in various aspects, are suitable for administration to a subject.

The present disclosure provides a pharmaceutical composition comprising a plurality of nanoparticles according to the present disclosure and a pharmaceutically acceptable carrier, diluent, or excipient. In various aspects, the composition comprises about 10¹⁰ nanoparticles per mL to about 10¹⁵ nanoparticles per mL, optionally about 10¹² nanoparticles ± 10% per mL.

A method of inducing or increasing an immune response against a SARS-CoV-2 virus in a subject is provided by the present disclosure. In exemplary embodiments, the method comprises administering to the subject the pharmaceutical composition of the present disclosure. In exemplary aspects, the nucleic acid molecules are mRNA. Optionally, the composition is systemically administered to the subject. For example, the composition is administered intravenously. In various aspects, the pharmaceutical composition is administered in an amount which is effective to activate dendritic cells (DCs) in the subject. In various instances, the immune response is a T cell-mediated immune response. Optionally, the T cell-mediated immune response comprises activity by tumor infiltrating lymphocytes (TILs).

The present disclosure also provides a method of delivering RNA molecules to lungs of a subject. The present disclosure also provides a method of delivering RNA molecules to a lymph node and/or a reticuloendothelial organ (e.g., spleen or liver). In exemplary embodiments, the method comprises administering to the subject a presently disclosed pharmaceutical composition.

A method of treating a subject with a disease or disorder is furthermore provided herein. In exemplary embodiments, the method comprises administering to the subject a pharmaceutical composition of the present disclosure in an amount effective to treat the disease or disorder in the subject. In various instances, the subject has COVID-19 or is at risk of infection with SARS-CoV-2.

Additional embodiments and aspects of the presently disclosed pharmaceutical compositions and methods are provided below.

### BRIEF DESCRIPTION OF THE DRAWINGS

FIG. 1A is a series of illustrations of a lipid bilayer, liposome and a general scheme leading to multilamellar (ML) RNA NPs (boxed).

FIG. 1B is a pair of CEM images of uncomplexed NPs (left) and ML RNA NPs (right).

FIG. 2A is an illustration of a general scheme leading to cationic RNA lipoplexes (LPX).

FIG. 2B is an illustration of a general scheme leading to anionic RNA lipoplexes.

FIGS. 2C-2E are CEM images. FIG. 2C is a CEM image of uncomplexed NPs; FIG. 2D is a CEM image of RNA LPXs; and FIG. 2E is a CEM image of ML RNA NPs.

FIG. 2F is a graph of the % CD86+ of CD11c+MHC Class II+ splenocytes present in the spleens of mice treated with ML RNA NPs (ML RNA-NPs), RNA LPXs, anionic LPXs, or of untreated mice.

FIG. 2G is a graph of the % CD44+CD62L+ of CD8+ splenocytes present in the spleens of mice treated with ML RNA NPs (ML RNA-NPs), RNA LPXs, anionic LPXs, or of untreated mice.

FIG. 2H is a graph of the % CD44+CD62L of CD4+ splenocytes present in the spleens of mice treated with ML RNA NPs (ML RNA-NPs), RNA LPXs, anionic LPXs, or of untreated mice.

FIG. 2I is a graph of the % survival of mice treated with ML RNA NPs (ML RNA-NPs), RNA LPXs, anionic LPXs, or of untreated mice.

FIG. 2J is a graph of the amount of IFN-α produced in mice upon treatment with ML RNA NPs (ML RNA-NPs), RNA LPXs, anionic LPXs, or of untreated mice.

FIG. 3A is a pair of photographs of lungs of mice treated with ML RNA NPs or of untreated mice.

FIG. 3B is a graph of the % central memory T cells (CD62L+CD44+ of CD3+ cells) present in mice treated with ML RNA NPs loaded with tumor specific RNA or with ML RNA NPs with non-specific RNA (GFP RNA) or of untreated mice.

FIG. 3C is a graph of the % survival of mice treated with ML RNA NPs loaded with tumor specific RNA or with ML RNA NPs with non-specific RNA (GFP RNA) or of untreated mice.

FIG. 3D is a graph of the % survival of mice treated with ML RNA NPs loaded with tumor specific RNA or with ML RNA NPs with non-specific RNA (GFP RNA) or of untreated mice. This model is different from the one used to obtain the data of FIG. 3C.

FIGS. 4A-4D are graphs. FIG. 4A is a graph of the % expression of CD8 or CD44 and CD8 of CD3+ cells plotted as a function of time post administration of ML RNA NPs. FIG. 4B is a graph of the % expression of PDL1, MHC II, CD86 or CD80 of CD1 1 c+ cells plotted as a function of time post administration of ML RNA NPs. FIG. 4C is a graph of the % expression of CD44 and CD8 of CD3+ cells plotted as a function of time post administration of ML RNA NPs. FIG. 4D is a graph of the % survival of a canine treated with ML RNA NPs compared to the median survival (dotted line).

FIG. 5 is a CEM image of ML RNA NPs with arrows pointing to examples with several layers.

FIG. 6 is a cartoon delineating the generation of personalized ML RNA NP, using tumor mRNA loaded NPs as an illustrative example. From as few as 100-500 biopsied brain tumor cells, total RNA is extracted and a cDNA library is generated from which copious amounts of mRNA (representing a personalized tumor specific transcriptome) can be amplified. Negatively charged mRNA is then encapsulated into positively charged lipid NPs. NPs encapsulate RNA through electrostatic interaction and are administered intravenously (iv) for uptake by dendritic cells (DCs) in reticuloendothelial organs (i.e., liver spleen and lymph nodes). The RNA is then translated and processed by a DC’s intracellular machinery for presentation of peptides onto MHC Class I and II molecules, which activate CD4 and CD8+ T cells.

FIGS. 7A and 7B relate to an in vivo study. FIG. 7A is a timeline of the long-term survivor treatment. First and Second tumor inoculations are shown. FIG. 7B is a graph of the percent survival of animals after the second tumor inoculation for each of the three groups of mice: two groups treated before 2^{nd} tumor inoculation with ML RNA NPs comprising non-specific RNA (RNA not specific to the tumor in the subject; Green Fluorescence Protein (GFP) or pp65) and one group treated before 2^{nd} tumor inoculation with ML RNA NPs comprising tumor specific RNA or untreated animals prior to 2^{nd} tumor inoculation. Control group survival percentage is noted as “Untreated”.

FIG. 8 is a series of images depicting the localization of anionic LPX in mice upon administration.

FIG. 9 is an illustration of different RNA loaded lipid-NP formulations. Lipid-NPs or liposomes have been developed to protect nucleic acid delivery in vivo. (A) RNA lipoplexes were first developed with mRNA preserved in the lipid core and a net positive charge located on the outer surface. (B) Anionic lipoplexes with an excess of RNA tethered to the surface of bi-lamellar liposomes. (C) Multi-lamellar RNA-NPs with several layers of mRNA contained inside a tightly coiled liposome with alternating layers of positive/negative charge. Existing NP designs restrict amount of RNA packaging due to repellent charge in core and limited surface area. The multi-lamellar RNA-NPs package greater RNA per particle, boosting immune response against desired target.

FIG. 10 demonstrates multi-lamellar RNA NPs form complex structures that coil mRNA into multi-lamellar vesicles enhancing payload delivery. The bar graph illustrates gene expression (luminescence) for anionic RNA-LPS (first bar on left), RNA-lipoplex (second bar), RNA-NPs (lo) (third bar), and RNA-NPs (high) (fourth bar).

FIG. 11 demonstrates multi-lamellar RNA NPs mediate increased DC activation and IFN-α release. RNA/anionic lipoplex (LPX) or RNA-NPs were i.v. (intravenously) administered once weekly (x3) to C57BI/6 mice, and spleens were harvested one week later for assessment of activated DCs (left). Serum was drawn 6h after the initial treatment for IFN-α assessment by ELISA (right).

FIG. 12 demonstrates multi-lamellar RNA-NPs are superior to LPX and peptide based vaccines in eliciting antigen specific T cells. RNA/anionic lipoplex (LPX) (left) or peptide based vaccines (right) formulated in complete Freund’s adjuvant (CFA) were compared with OVA specific RNA-NPs. Animals (n=5-8/group) received 10⁷ OT-Is before assessment of tetramer positive (OVA specific) T cells one week after last vaccine.

FIG. 13 demonstrates RNA-NPs induce memory re-stimulation response against CMV matrix protein pp65. Weekly pp65 RNA-NPs (x3) were administered to naïve C57/BI/6 mice, and splenocytes were harvested one week later for culture with overlapping pp65 peptide pool and assessment of IFN-y (*p<0.05, **p<0.01, Mann Whitney).

FIG. 14 demonstrates multi-lamellar tumor specific mRNA-NPs mediate superior efficacy. Different lipoplexes (LPX) or RNA-NPs were loaded with tumor specific mRNA and compared in a therapeutic lung cancer model (K7M2) (n=8/group). Each vaccine was iv administered weekly (x3), **p<0.01, Gehan-Wilcoxon test.

FIG. 15 demonstrates RNA-NPs elicit near immediate IFN-a surge, PBMC margination, and peripheral DC activation in canines. Within 2 h of initial RNA-NP administration, an increase in IFN-α (left), initial decrease in PBMCs (middle, suggesting lymph node honing for antigen education before egress), and activation of CD11c+ DCs (right) was observed.

FIG. 16 demonstrates RNA-NPs improve median survival in canines with terminal gliomas, demonstrating the ability of the composition described herein to elicit a clinically meaningful immune response in vivo. Survival of canines (boxer breed) age 9-11 years (n=5) diagnosed with terminal gliomas receiving only supportive care and weekly tumor RNA-NPs (x3) (following tumor biopsy without resection). Median survival shown as dotted line (29 days) is reported from a previous study of cerebral astrocytomas in canines receiving only supportive care. Mean survival from separate studies ranges between 60-80 days. Median one-sided exact log rank test p-value distribution with bootstrapping to account for uncertainty: *p=0.021.

FIG. 17 demonstrates GLP Toxicity study results of tumor specific RNA-NPs against KR158b. Intravenous injection into the tail vein of mice at 1.0 mg/kg KR158 and p65 mRNAs + 15.0 mg/kg lipid NP on Study Days 0, 14 and 28 resulted in no gross or microscopic test article related findings.

FIGS. 18A-18D demonstrate charge modified RNA-NPs can be directed to, e.g., the lung or the spleen. FIG. 18A: Cationic (~ +40 mV, left) versus anionic (~ -30 mV, right) RNA-NPs encoding for luciferase were administered to Balb/c mice by i.v. tail vein injection. Mice were injected i.p. with luciferin 6 h after RNA-NPs and imaged for bioluminescence by IVIS imaging. The ability to manipulate RNA-NPs for lung localization is especially relevant for COVID-19 protection. FIGS. 18B-18D: RNA-NPs were injected iv into C57BI/6 mice (n=3-4/group). Reticuloendothelial organs (lymph nodes, spleens, and livers) were harvested within 24 h for assessment of CD11c cells expressing activation marker CD86 (*p<0.05, **p<0.01, Mann-Whitney test) from lymph nodes (FIG. 18B), splenocytes (FIG. 18C), or liver cells (FIG. 18D). The data establish that the constructs of the disclosure can be leveraged for near immediate pulmonary protection against, e.g., viral pathogens (such as coronavirus) with only a single administration.

FIG. 19 is an illustration of an exemplary mRNA comprising a nucleotide sequence encoding a SARS-CoV-2 protein (e.g., spike, membrane, envelope, nucleocapsid) and a poly(A) tail.

FIG. 20 is a table of sequences provided in the Sequence Listing.

FIGS. 21A and 21B are graphs illustrating the results of the study of Example 11. FIG. 21A is a graph of the number of effector memory T cells (CD3+CD8+CD44+ cells per well) upon re-stimulation with SARS-CoV-2 Spike peptide (Spike peptide restim - empty circles) or without re-stimulation (unstimulated, gray filled circles). Mice were administered multilamellar (ML) RNA nanoparticles (NPs) loaded with mRNA encoding the full-length Spike protein of SARS-CoV-2. The mice received a total of 3 vaccinations within a week. PMBCs were harvested from the mice and then tested for the number of effector memory T cells responsive to SARS-CoV-2 Spike protein. FIG. 21B is a graph illustrating MIP1-a released (pg/mL) from unstimulated cells (empty bar) and cells re-stimulated with spike peptide (solid bar).

FIGS. 22A-C provide sequences of the SARS-CoV-2 Spike protein relevant to the nanoparticles of the present disclosure (FIGS. 22A and 22C) as well as a vector map (FIG. 22B).

FIG. 23 is inclusion criteria for subjects in the study of Example 14.

FIG. 24 is exclusion criteria for subjects in the study of Example 14.

FIG. 25 is a calendar for patient assessments referenced in the Examples.

FIG. 26 is a bar graph comparing SARS-CoV-2 IgG (absorbance) (y-axis) of untreated subjects and RNA-NP-treated subjects (x-axis) C57BI/6 mice were vaccinated with weekly RNA-NPs encoding full-length SARS-CoV-2 spike mRNA. Serum was harvested at one month for assessment of spike specific antibodies by ELISA.

FIGS. 27A-27B are bar graphs illustrating effector memory T cell expansion (FIG. 27A) and MIP-1a production (FIG. 27B) following spike peptide restimulation. Mice receiving SARS-CoV-2 spike RNA-NPs had more effector memory T cells after vaccination with significant memory recall expansion. SARS-CoV-2 full-length spike mRNA loaded into NPs were i.v. administered into naïve C57BI/6 mice (n=7-8) and blood was harvested on ~day10. Peripheral blood mononuclear cells (PBMCs) were left unstimulated or re-stimulated with 200 ng of overlapping peptide mix (PepMix™ SARS-CoV-2 (Spike Glycoprotein), JPT peptides) of 15mers with 11aa overlap from SARS-CoV-2 spike glycoprotein. (FIG. 27A) Cells were cultured for 36 hours before harvest and staining for effector memory cells by CD3+CD8+CD44+ subsets. These were compared with PBMCs from untreated mice. (FIG. 27B) Supernatants were harvested and assessed for multiplex cytokine detection by Eve Technologies. (*p<0.05, **p<0.01, ***p<0.001, Mann-Whitney test).

FIG. 28 is a bar graph illustrating IFN-γ production following spike protein restimulation. Mice receiving SARS-CoV-2 spike RNA-NPs demonstrate memory recall response ex vivo. Weekly SARS-CoV-2 full-length spike mRNA loaded into NPs were administered to naïve C57BI/6 mice (n=7-8) i.v. and blood was harvested on day 16. PBMCs were left unstimulated or re-stimulated with cell line of DC2.4s electroporated with SARS-CoV-2 spike mRNA and compared with PBMCs from untreated mice. Supernatants were collected for IFN-y analysis (*p<0.05, Mann-Whitney).

FIGS. 29A and 29B illustrate that 5′ and 3′ UTRs for alpha globin enhance percentage of tetramer+ T cells and long-term survival benefit. RNA-NPs encoding for OVA mRNA with 5′ and 3′ UTRs for alpha-globin were administered i.v. to naïve C57BI/6 mice with OT-I T cells, and spleens were harvested a week later for assessment of tetramer+ T cells. Compared with unmodified OVA RNA-NPs, alpha globin modified RNA-NPs increase percentage of antigen specific T cells. FIG. 29A is a graph comparing % OVA Tetramer-positive CD8+ CD3+ cells from subjects treated with NP, RNA-NP, and RNA-NP with UTRs. FIG. 29B is a graph illustrating percent survival in these groups following tumor implantation. The UTR RNA-NP treated subjects demonstrated enhanced survival (about 50% to 35 days post implantation).

FIG. 30 illustrates that RNA-NPs with HCV’s 5′ IRES and 3′polyUUU tail increase antigen specific T cells. RNA-NPs encoding OVA mRNA and comprising mRNAs containing HCV’s 5′ IRES and 3′polyUUU tail were administered i.v. to naïve C57BI/6 mice (n=3-4/group). OT-I T cells and spleens were harvested a week later for assessment of tetramer+ T cells. HCV modified RNA-NPs increase percentage of antigen specific T cells.

FIG. 31 illustrates that full-length LAMP conjugated pp65 appears to induce greater percentage of antigen specific T cells. Full length LAMP conjugated RNA for pp65 was i.v. administered to naïve mice (n=5/group) once weekly (x3) and spleens were harvested for re-stimulation with overlapping pp65 peptide pool (*p<0.05, Mann-Whitney test). The graph compares IFN-฀ production in subjects administered NP alone, RNA-NP, or LAMP RNA-NP.

FIGS. 32A and 32B are graphs illustrating % OVA specific Tetramer+ CD8 cells in subjects administered NP alone and RNA-NP in MDAS knock-out subjects. FIGS. 32A - T cells alone; FIG. 32B -following restimuation assay with B16F10-OVA. While wishing to be bound by any particular theory, immunogenicity of multi-lamellar RNA-NPs appears to be dependent on intracellular pathogen recognition receptors such as MDA-5, in contrast to existing mRNA nanolipid platforms.

### DETAILED DESCRIPTION

The present disclosure relates to nanoparticles comprising a cationic lipid and nucleic acids. As used herein the term “nanoparticle” refers to a particle that is less than about 1000 nm in diameter. As the nanoparticles of the present disclosure comprise cationic lipids that have been processed to induce liposome formation, the presently disclosed nanoparticles in various aspects comprise liposomes. Liposomes are artificially-prepared vesicles which, in exemplary aspects, are primarily composed of a lipid bilayer. Liposomes in various instances are used as a delivery vehicle for the administration of nutrients and pharmaceutical agents. In various aspects the liposomes of the present disclosure are of different sizes and the composition may comprise one or more of (a) a multilamellar vesicle (MLV) which may be hundreds of nanometers in diameter and may contain a series of concentric bilayers separated by narrow aqueous compartments, (b) a small unicellular vesicle (SUV) which may be smaller than 50 nm in diameter, and (c) a large unilamellar vesicle (LUV) which may be between 50 and 500 nm in diameter. Liposomes in various instances are designed to comprise opsonins or ligands in order to improve the attachment of liposomes to unhealthy tissue or to activate events such as, but not limited to, endocytosis. In exemplary aspects, liposomes contain a low or a high pH in order to improve the delivery of the pharmaceutical formulations. In various instances, liposomes are formulated depending on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In exemplary embodiments, the nanoparticle comprises a surface and an interior comprising (i) a core and (ii) at least two nucleic acid layers, optionally, more than two nucleic acid layers. In exemplary instances, each nucleic acid layer is positioned between a lipid layer, e.g., a cationic lipid layer. In exemplary aspects, the nanoparticles are multilamellar comprising alternating layers of nucleic acid and lipid. In exemplary embodiments, the nanoparticle comprises at least three nucleic acid layers, each of which is positioned between a cationic lipid bilayer. In exemplary aspects, the nanoparticle comprises at least four or five nucleic acid layers, each of which is positioned between a cationic lipid bilayer. In exemplary aspects, the nanoparticle comprises at least more than five (e.g., 6, 7, 8, 9, 10, 11, 12, 13, 14, 15, 16, 17, 18, 19, 20, or more) nucleic acid layers, each of which is positioned between a cationic lipid bilayer. As used herein, the term “cationic lipid bilayer” is meant a lipid bilayer comprising, consisting essentially of, or consisting of a cationic lipid or a mixture thereof. Suitable cationic lipids are described herein. As used herein the term “nucleic acid layer” is meant a layer of the presently disclosed nanoparticle comprising, consisting essentially of, or consisting of a nucleic acid, e.g., RNA.

The unique structure of the nanoparticle of the present disclosure results in mechanistic differences in how the multilamellar nanoparticles (ML-NPs) exert a biological effect. Previously described RNA-based nanoparticles exert their effect, at least in part, through the toll-like receptor 7 (TLR7) pathway. Surprisingly, the multi-lamellar nanoparticles of the instant disclosure mediate efficacy independent of TLR7. While not wishing to be bound to any particular theory, intracellular pathogen recognition receptors (PRRs), such as MDA-5, appear more relevant to biological activity of the multi-lamellar nanoparticles than TLRs. This likely allows ML RNA-NPs to stimulate multiple intracellular PRRs (i.e., RIG-I, MDA-5) as opposed to singular TLRs (i.e., TLR7 in the endosome) culminating in greater release of type I interferons and induction of more potent innate immunity. This allows RNA-NPs to demonstrate superior efficacy with long-term survivor benefit.

In various aspects, the presently disclosed nanoparticle comprises a positively-charged surface. In some instances, the positively-charged surface comprises a lipid layer, e.g., a cationic lipid layer. In various aspects, the outermost layer of the nanoparticle comprises a cationic lipid bilayer. Optionally, the cationic lipid bilayer comprises DOTAP. In various instances, the surface comprises a plurality of hydrophilic moieties of the cationic lipid of the cationic lipid bilayer. In some aspects, the core comprises a cationic lipid bilayer. In some instances, the outermost region of the core comprise a cationic lipid bilayer comprising DOTAP. In various instances, the core lacks nucleic acids, optionally, the core comprises less than about 0.5 wt% nucleic acid.

In exemplary aspects, the nanoparticle has a diameter within the nanometer range and accordingly in certain instances are referred to herein as “nanoliposomes” or “liposomes.” In exemplary aspects, the nanoparticle has a diameter between about 50 nm to about 500 nm, e.g., about 50 nm to about 450 nm, about 50 nm to about 400 nm, about 50 nm to about 350 nm, about 50 nm to about 300 nm, about 50 nm to about 250 nm, about 50 nm to about 200 nm, about 50 nm to about 150 nm, about 50 nm to about 100 nm, about 100 nm to about 500 nm, about 150 nm to about 500 nm, about 200 nm to about 500 nm, about 250 nm to about 500 nm, about 300 nm to about 500 nm, about 350 nm to about 500 nm, or about 400 nm to about 500 nm. In exemplary aspects, the nanoparticle has a diameter between about 50 nm to about 300 nm, e.g., about 100 nm to about 250 nm, about 110 nm ±5 nm, about 115 nm ±5 nm, about 120 nm ±5 nm, about 125 nm ±5 nm, about 130 nm ±5 nm, about 135 nm ±5 nm, about 140 nm ±5 nm, about 145 nm ±5 nm, about 150 nm ±5 nm, about 155 nm ±5 nm, about 160 nm ±5 nm, about 165 nm ±5 nm, about 170 nm ±5 nm, about 175 nm ±5 nm, about 180 nm ±5 nm, about 190 nm ±5 nm, about 200 nm ±5 nm, about 210 nm ±5 nm, about 220 nm ±5 nm, about 230 nm ±5 nm, about 240 nm ±5 nm, about 250 nm ±5 nm, about 260 nm ±5 nm, about 270 nm ±5 nm, about 280 nm ±5 nm, about 290 nm ±5 nm, or about 300 nm ±5 nm. In exemplary aspects, the nanoparticle is about 50 nm to about 250 nm in diameter. In some aspects, the nanoparticle is about 70 nm to about 200 nm in diameter.

In exemplary aspects, the nanoparticle is present in a pharmaceutical composition comprising a heterogeneous mixture of nanoparticles ranging in diameter, e.g., about 50 nm to about 500 nm or about 50 nm to about 250 nm in diameter. Optionally, the pharmaceutical composition comprises a heterogeneous mixture of nanoparticles ranging from about 70 nm to about 200 nm in diameter.

In exemplary instances, the nanoparticle is characterized by a zeta potential of about +40 mV to about +60 mV, e.g., about +40 mV to about +55 mV, about +40 mV to about +50 mV, about +40 mV to about +50 mV, about +40 mV to about +45 mV, about +45 mV to about +60 mV, about +50 mV to about +60 mV, or about +55 mV to about +60 mV. In exemplary aspects, the nanoparticle has a zeta potential of about +45 mV to about +55 mV. The nanoparticle in various instances has a zeta potential of about +50 mV. In various aspects, the zeta potential is greater than +30 mV or +35 mV. The zeta potential is one parameter which distinguishes the nanoparticles of the present disclosure and those described in Sayour et al., Oncoimmunology 6(1): e1256527 (2016).

In exemplary embodiments, the nanoparticles comprise a cationic lipid. In some embodiments, the cationic lipid is a low molecular weight cationic lipid such as those described in U.S. Pat. Application No. 20130090372, the contents of which are herein incorporated by reference in their entirety. The cationic lipid in exemplary instances is a cationic fatty acid, a cationic glycerolipid, a cationic glycerophospholipid, a cationic sphingolipid, a cationic sterol lipid, a cationic prenol lipid, a cationic saccharolipid, or a cationic polyketide. In exemplary aspects, the cationic lipid comprises two fatty acyl chains, each chain of which is independently saturated or unsaturated. In some instances, the cationic lipid is a diglyceride. For example, in some instances, the cationic lipid may be a cationic lipid of Formula I or Formula II:

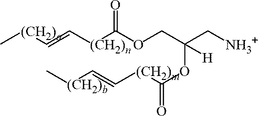

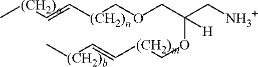

wherein each of a, b, n, and m is independently an integer between 2 and 12 (e.g., between 3 and 10). In some aspects, the cationic lipid is a cationic lipid of Formula I wherein each of a, b, n, and m is independently an integer selected from 3, 4, 5, 6, 7, 8, 9, and 10. In exemplary instances, the cationic lipid is DOTAP (1,2-dioleoyl-3-trimethylammonium-propane), or a derivative thereof. In exemplary instances, the cationic lipid is DOTMA (1,2-di-O-octadecenyl-3-trimethylammonium propane), or a derivative thereof.

In some embodiments, the nanoparticles comprise liposomes formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety). In some embodiments, the nanoparticles comprise liposomes formed from the synthesis of stabilized plasmid-lipid particles (SPLP) or stabilized nucleic acid lipid particle (SNALP) that have been previously described and shown to be suitable for oligonucleotide delivery in vitro and in vivo. The nanoparticles in some aspects are composed of 3 to 4 lipid components in addition to the nucleic acid molecules. In exemplary aspects, the liposome comprises 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al., Pharm Res. 2005; 22(3):362-72. In exemplary instances, the liposome comprises 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al., J. Control Release 2005; 107(2): 276-87.

In some embodiments, the liposomes comprise from about 25.0% cholesterol to about 40.0% cholesterol, from about 30.0% cholesterol to about 45.0% cholesterol, from about 35.0% cholesterol to about 50.0% cholesterol, and/or from about 48.5% cholesterol to about 60% cholesterol. In some embodiments, the liposomes may comprise a percentage of cholesterol selected from the group consisting of 28.5%, 31.5%, 33.5%, 36.5%, 37.0%, 38.5%, 39.0%, and 43.5%. In some embodiments, the liposomes may comprise from about 5.0% to about 10.0% DSPC and/or from about 7.0% to about 15.0% DSPC.

In some embodiments, the liposomes are DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel).

In various instances, the cationic lipid comprises 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

The liposome in various aspects comprises DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some aspects, the liposome comprises a cationic lipid such as, but not limited to, DLin-DMA, DLin-D-DMA, DLin-MC3-DMA, DLin-KC2-DMA, DODMA and amino alcohol lipids. The amino alcohol cationic lipid comprises in some aspects lipids described in and/or made by the methods described in U.S. Pat. Publication No. US20130150625, herein incorporated by reference in its entirety. As a non-limiting example, the cationic lipid in certain aspects is 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

In various embodiments, the liposome comprises (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of about 20-60% cationic lipid: 5-25% neutral lipid: 25-55% sterol; 0.5-15% PEG-lipid.

In some embodiments, the liposome comprises from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about 35 to about 65%, from about 45 to about 65%, about 60%, about 57.5%, about 50%, or about 40% on a molar basis.

In some embodiments, the liposome comprises from about 0.5% to about 15% on a molar basis of the neutral lipid e.g., from about 3 to about 12%, from about 5 to about 10% or about 15%, about 10%, or about 7.5% on a molar basis. Examples of neutral lipids include, but are not limited to, DSPC, POPC, DPPC, DOPE and SM. In some embodiments, the nanoparticle does not comprise a neutral lipid. In some embodiments, the formulation includes from about 5% to about 50% on a molar basis of the sterol (e.g., about 15 to about 45%, about 20 to about 40%, about 40%, about 38.5%, about 35%, or about 31% on a molar basis). An exemplary sterol is cholesterol. In some embodiments, the nanoparticle does not comprise a sterol, such as cholesterol. In some embodiments, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis). In some embodiments, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Examples of PEG-modified lipids include, but are not limited to, PEG-distearoyl glycerol (PEG-DMG) (also referred herein as PEG-C14 or C14-PEG), PEG-cDMA (further discussed in Reyes et al. J. Controlled Release, 107, 276-287 (2005) the contents of which is herein incorporated by reference in its entirety).

In exemplary aspects, the cationic lipid may be selected from (20Z,23Z)-N,N-dimethylnonacosa-20,23-dien-10-amine, (17Z,20Z)-N,N-dimemylhexacosa-17,20-dien-9-amine, (1Z,19Z)—N,N-dimethylpentacosa-16,19-dien-8-amine, (13Z,16Z)-N,N-dimethyldocosa-13,16-dien-5-amine, (12Z,15Z)-N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)-N,N-dimethylheptacosa-18,21-dien-10-amine, (15Z,18Z)-N,N-dimethyltetracosa-15,18-dien-5-amine, (14Z, 17Z)—N,N-dimethyltricosa-14,17-dien-4-amine, (19Z,22Z)— N,N-dimeihyloctacosa-19,22-dien-9-amine, (18Z,21Z)—N,N-dimethylheptacosa-18,21-dien-8-amine, (17Z,20Z)—N, N-dimethylhexacosa-17,20-dien-7-amine, (16Z, 19Z)—N, N-dimethylpentacosa-16, 19-dien-6-amine, (22Z,25Z)-N,N-dimethylhentriaconta-22,25-dien-10-amine, (21Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine,1-[(11Z,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N, N-dimethylnonacos-17-en-10-amine, (24Z)—N, N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z, 15Z)—N,N-dimethyl-2-nonylhenicosa-12, 15-dien-1-amine, (13Z,16Z)— N,N-dimethyl-3-nonyldocosa-13,16-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine,N,N-dimethyl-1-[(1S,2S)-2-{[(1R,2R)-2-pentylcyclopropyl]methyl}cyclopropyl]nonadecan-10-amine, N, N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1 -yloxy]propan-2-amine, 1-{2-[(9Z, 12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N, N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)-N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxyl-3-[(9Z,12Z)-octadeca-9,12-dien-1-yloxy]propan-2-amine, (2R)-1 -[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)—N,N-dimethylnonacosa-11,20,2-trien-10-amine or a pharmaceutically acceptable salt or stereoisomer thereof.

In some embodiments, the nanoparticle comprises a lipid-polycation complex. The formation of the lipid-polycation complex may be accomplished by methods known in the art and/or as described in U.S. Pat. Pub. No. 20120178702, herein incorporated by reference in its entirety. As a non-limiting example, the polycation may include a cationic peptide or a polypeptide such as, but not limited to, polylysine, polyornithine and/or polyarginine. In some embodiments, the composition may comprise a lipid-polycation complex, which may further include a non-cationic lipid such as, but not limited to, cholesterol or dioleoyl phosphatidylethanolamine (DOPE).

In various aspects, the cationic liposomes optionally do not comprise a non-cationic lipid. Neutral molecules, in some aspects, may interfere with coiling/condensation of multi-lamellar NPs resulting in RNA/DNA loaded liposomes greater than 200 nm in size. Cationic liposomes generated without helper molecules can comprise a size of about 70-200 nm (or less). These constructs consist of a cationic lipid with negatively charged nucleic acid, and may be formulated in a sealed rotary vacuum evaporator which prevents oxidation of the particles (when exposed to the ambient environment). In this embodiment, the absence of a helper lipid optimizes mRNA coiling into tightly packaged multilamellar NPs where each NP contains a greater amount of nucleic acid per particle. Due to increased nucleic acid payload per particle, these multi-lamellar RNA-NPs drive significantly greater innate immune responses, which are a significant predictor of efficacy for modulating the immune system.

The presently disclosed nanoparticles comprise a positively-charged surface and an interior comprising (i) a core and (ii) at least two nucleic acid layers, wherein each nucleic acid layer is positioned between a cationic lipid bilayer. The nucleic acid layers comprise nucleic acid molecules.

In some aspects, the nucleic acid molecules are present at a nucleic acid molecule: cationic lipid ratio of about 1 to about 5 to about 1 to about 25, such as about 1 to about 5 to about 1 to about 20, optionally, about 1 to about 18, about 1 to about 17, about 1 to about 15, about 1 to about 10, or about 1 to about 7.5. As used herein, the term “nucleic acid molecule: cationic lipid ratio” is meant a mass ratio, where the mass of the nucleic acid molecule is relative to the mass of the cationic lipid. Also, in exemplary aspects, the term “nucleic acid molecule: cationic lipid ratio” is meant the ratio of the mass of the nucleic acid molecule, e.g., RNA, added to the liposomes comprising cationic lipids during the process of manufacturing the ML RNA NPs of the present disclosure. In exemplary aspects, the nanoparticle comprises less than or about 10 µg RNA molecules per 150 µg lipid mixture. In exemplary aspects, the nanoparticle is made by incubating about 10 µg RNA with about 150 µg liposomes. In alternative aspects, the nanoparticle comprises more RNA molecules per mass of lipid mixture. For example, the nanoparticle may comprise more than 10 µg RNA molecules per 150 µg liposomes. The nanoparticle in some instances comprises more than 15 µg RNA molecules per 150 µg liposomes or lipid mixture.

In various aspects, the nucleic acid molecules are RNA molecules, e.g., transfer RNA (tRNA), ribosomal RNA (rRNA), and/or messenger RNA (mRNA). In various aspects, the RNA molecules comprise tRNA, rRNA, mRNA, or a combination thereof. In various aspects, the RNA is total RNA isolated from a cell. In exemplary aspects, the RNA is total RNA isolated from a diseased cell, such as, for example, a tumor cell or a cancer cell or an infected cell. Methods of obtaining total tumor RNA is known in the art and described herein at Example 1, and represents an example of methods for obtaining nucleic acid for incorporation into nanoparticles.

In exemplary instances, the RNA molecules are mRNA. In various aspects, mRNA is in vitro transcribed mRNA. In various instances, the mRNA molecules are produced by in vitro transcription (IVT). Suitable techniques of carrying out IVT are known in the art. In exemplary aspects, an IVT kit is employed. In exemplary aspects, the kit comprises one or more IVT reaction reagents. As used herein, the term “in vitro transcription (IVT) reaction reagent” refers to any molecule, compound, factor, or salt, which functions in an IVT reaction. For example, the kit may comprise prokaryotic phage RNA polymerase and promoter (T7, T3, or SP6) with eukaryotic or prokaryotic extracts to synthesize proteins from exogenous DNA templates.

In exemplary aspects, the RNA is in vitro transcribed mRNA, wherein the in vitro transcription template is cDNA made from RNA extracted from a virus or infected cell.

The nanoparticles of the disclosure are useful, in various aspects, in the treatment of cancer, and in these embodiments the nanoparticle optionally comprises a mixture of RNA which is RNA isolated from a tumor of a human, optionally, a malignant brain tumor, optionally, a glioblastoma, medulloblastoma, diffuse intrinsic pontine glioma, or a peripheral tumor with metastatic infiltration into the central nervous system.

In various aspects, the RNA comprises a sequence encoding a poly(A) tail so that the in vitro transcribed RNA molecule comprises a poly(A) tail at the 3′ end. In various aspects, the method of making a nanoparticle comprises additional processing steps, such as, for example, capping the in vitro transcribed RNA molecules.

In various aspects, the presently disclosed nanoparticles comprise RNA encoding a protein expressed by a virus. In exemplary aspects, the RNA is mRNA encoding a protein expressed by a virus, e.g., a viral protein. In exemplary aspects, the viral protein is expressed by a virus of the order Nidovirales optionally, of the order Cornidovirinaea. In exemplary aspects, the viral protein is expressed by a virus of the Coronaviridae family, optionally, of the Orthocornoavirinae subfamily. In exemplary instances, the viral protein is expressed by a virus of the Betacoronavirus genus, optionally of the Sarbecovirus subfamily. In various aspects, the viral protein is expressed by the species SARS-CoV-2. SARS-CoV-2 is a positive-sense single-stranded RNA virus with genetic similarity to bat coronaviruses and was first isolated in January 2020 from patients in Wuhan, China (Hui et al., International J Infectious Diseases 91: 264-266 (2020)).

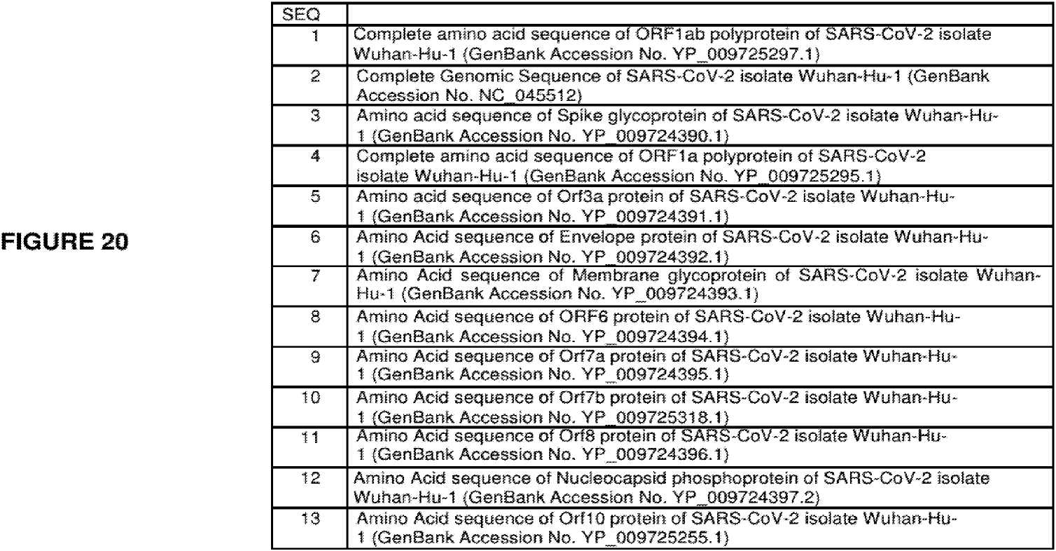

Optionally, the viral protein is one encoded and expressed by a SARS CoV-2 virus, e.g., a SARS CoV-2 protein. Complete genome sequences of SARS-CoV-2 are publicly available online at the GenBank database of the National Center for Biotechnology Information (NCBI) website (ncbi.nlm.nih.gov). Exemplary genome sequences include those having the following GenBank Accession numbers: MN988668, NC_045512), MN938384.1, MN975262.1, MN985325.1, MN988713.1, MN994467.1, MN994468.1, MN997409.1, MN988668. The complete genome sequence of GenBank Accession No. NC_045512, as well as the amino acid sequences of encoded proteins, are provided in the Sequence Listing provided herein. FIG. 20 provides a table listing various sequences of the sequence listing. In exemplary aspects, the SARS-CoV-2 protein is a spike protein, membrane protein, envelope protein, or nucleocapsid protein. In exemplary aspects, the SARS CoV-2 protein is a SARS-CoV-2 Spike (S) protein or a fragment thereof. In exemplary embodiments, the NPs of the present disclosure comprise a mixture of RNA molecules. In exemplary aspects, the mixture of RNA molecules comprises mRNAs encoding the S protein or a portion thereof. In exemplary instances, the mixture of RNA molecules comprises mRNAs encoding other proteins encoded by the SARS CoV-2 genome. Suitable SARS CoV-2 proteins include but are not limited to ORF1ab polyprotein (QIQ50091.1), ORF3a protein (QIQ50093.1), envelope protein (QIQ50094.1), membrane glycoprotein (QIQ50095.1), ORF6 protein (QIQ50096.1), ORF7a protein (QIQ50097.1), ORF8 protein (QIQ50098.1), nucleocapsid protein (QIQ50099.1), and ORF10 protein (QIQ50100.1).

In various instances, the mRNA comprises a nucleotide sequence encoding an amino acid sequence set forth in the Sequence Listing. In various aspects, the nucleotide sequence encodes an amino acid sequence which has at least 50%, at least 60%, at least 70%, at least 80%, at least 85%, at least 90% or has greater than 90% sequence identity (e.g., about 91%, about 92%, about 93%, about 94%, about 95%, about 96%, about 97%, about 98%, about 99%) to an amino acid sequence set forth in the Sequence Listing. In various aspects, the nucleotide sequence encodes an amino acid sequence set forth in the Sequence Listing with 1 to 10 amino acid substitutions, e.g., about 1 to 9, about 1 to 8, about 1 to 7, about 1 to 6, about 1 to 5, about 1 to 4, about 1 to 3, about 1 to 2 amino acid substitutions or 1, 2, 3, 4, 5, 6, 7, 8, 9, 10 amino acid substitutions. Alternatively, the mRNA comprises a nucleotide sequence provided in the Sequence Listing. In various aspects, the nucleotide sequence of the NP payload has at least 50%, at least 60%, at least 70%, at least 80%, at least 85%, at least 90% or has greater than 90% sequence identity (e.g., about 91%, about 92%, about 93%, about 94%, about 95%, about 96%, about 97%, about 98%, about 99%) to a nucleotide sequence set forth in the Sequence Listing. Alternatively, the mRNA is encoded by a DNA sequence set forth in the Sequence Listing.

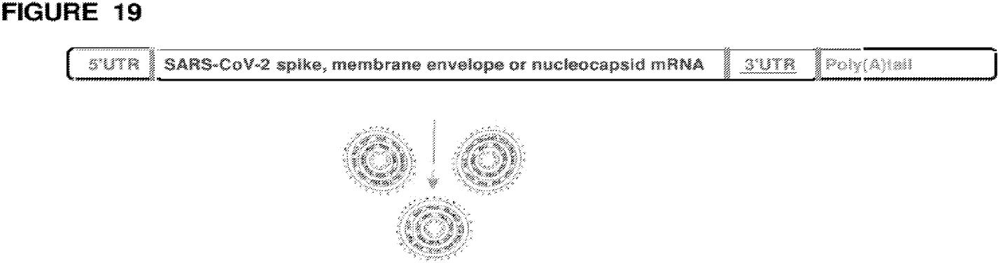

In various aspects, the nanoparticle comprises RNA molecules comprising a nucleotide sequence encoding a viral protein, or a mixture of viral proteins, and a poly(A) tail at the 3′ end. In various instances, the nanoparticle comprises RNA molecules as essentially depicted in FIG. 19, optionally comprising the sequences referenced in FIG. 20.

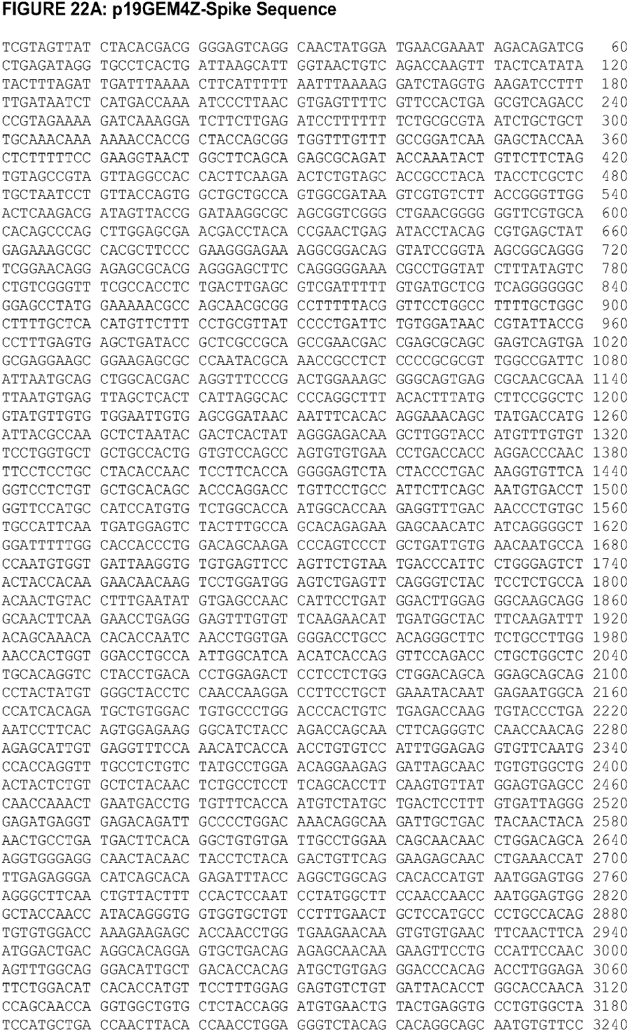

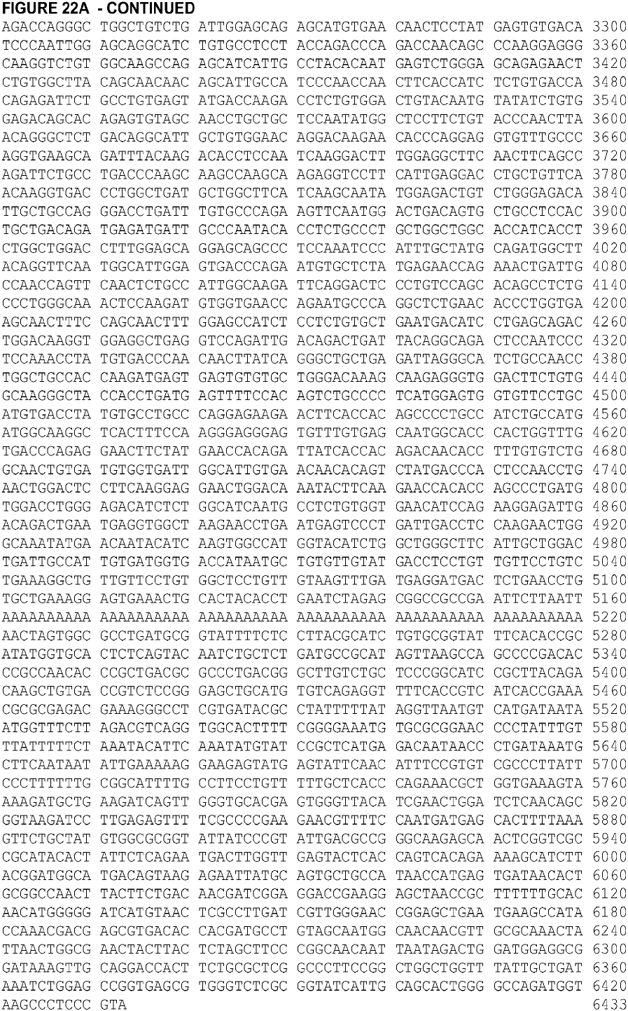

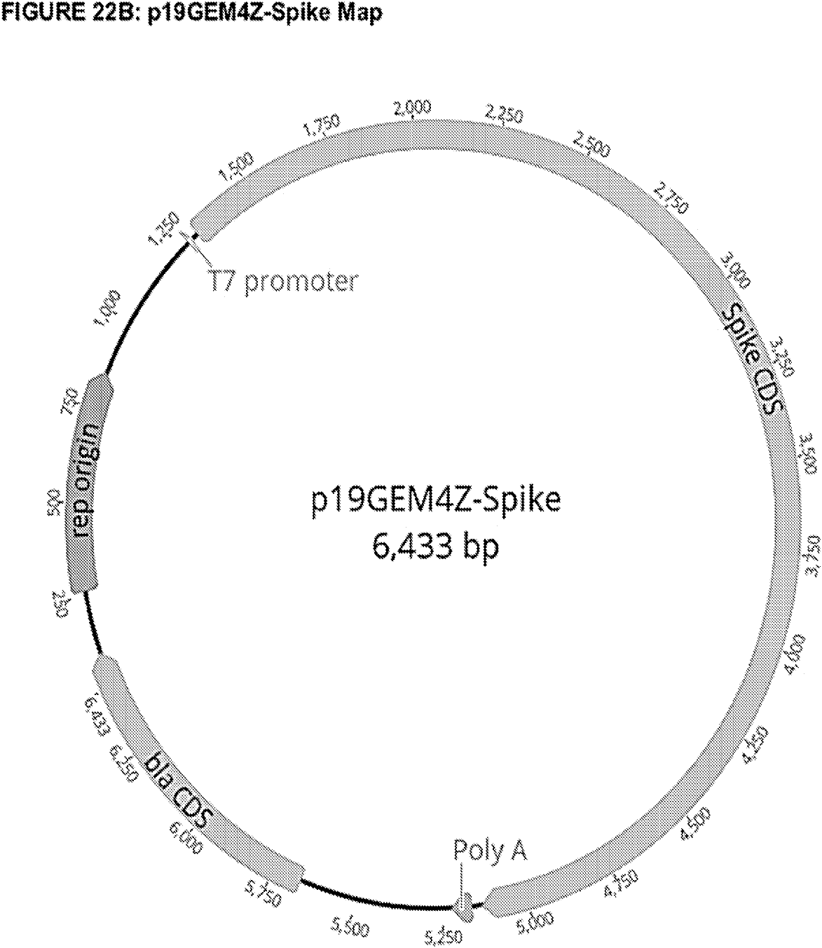

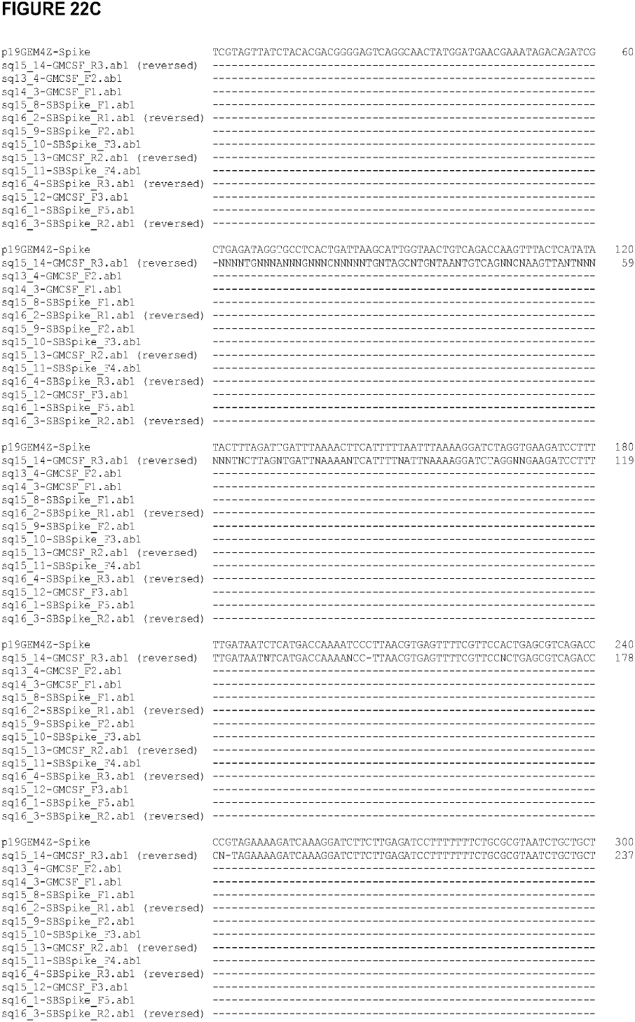

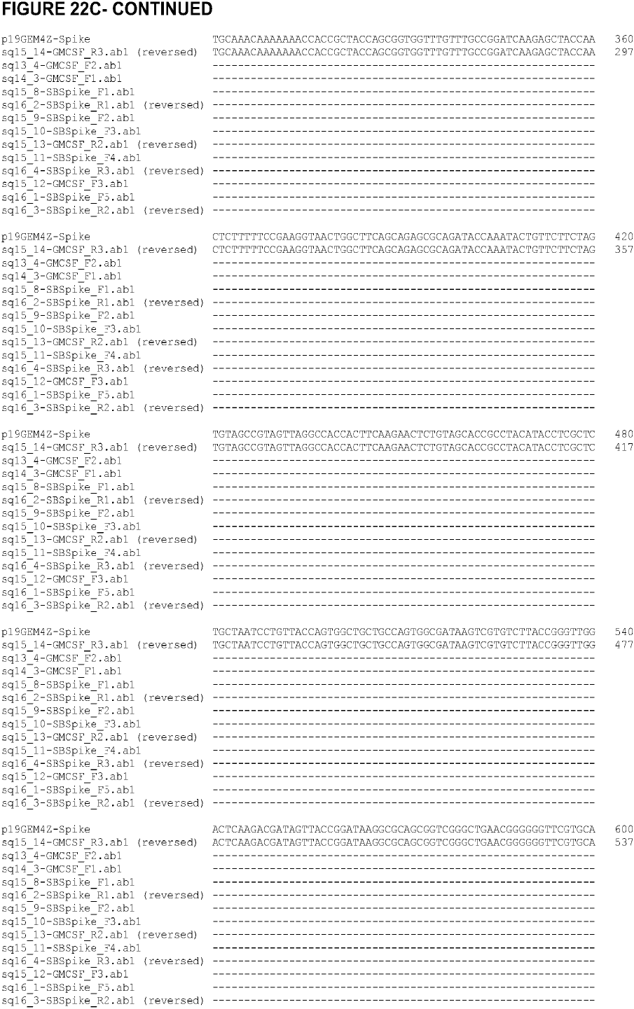

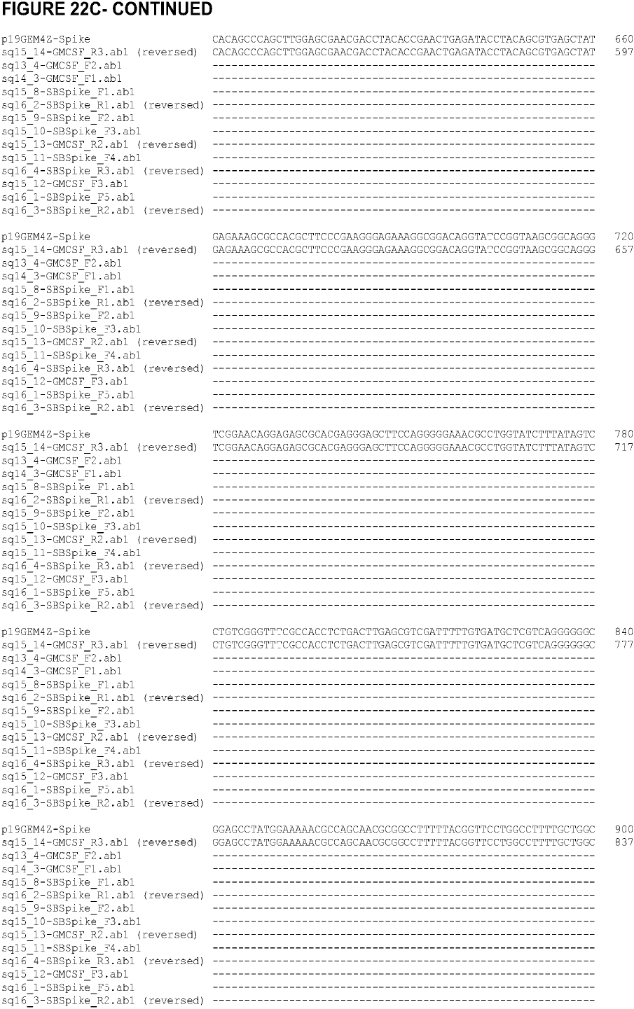

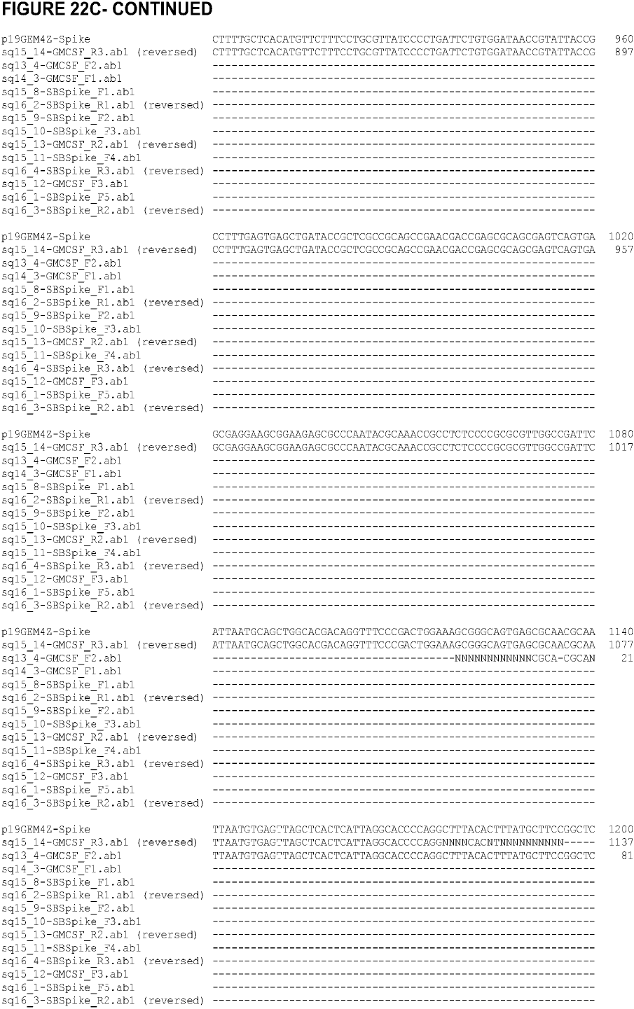

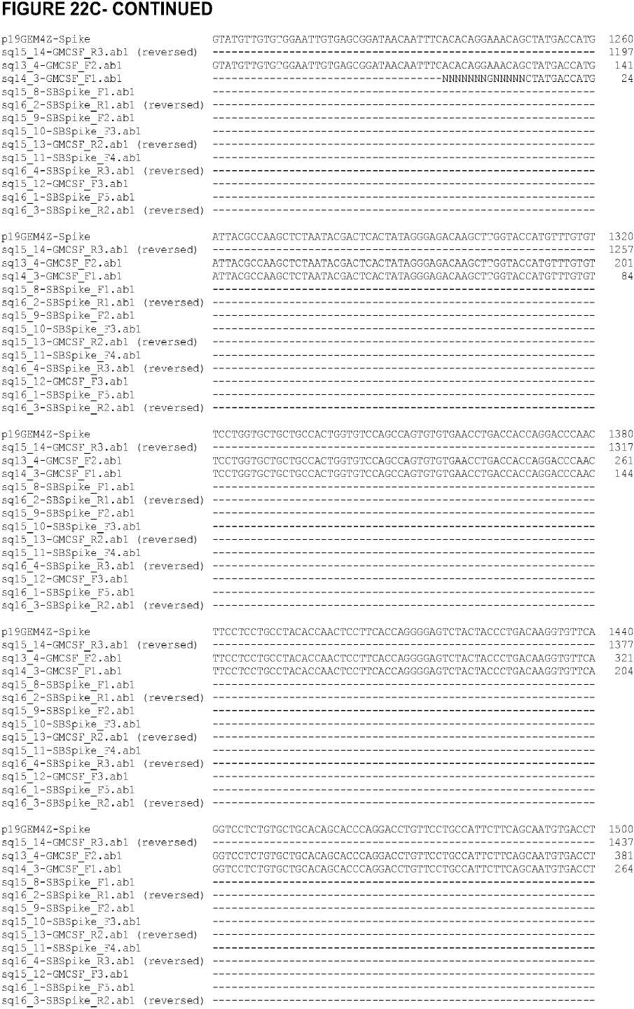

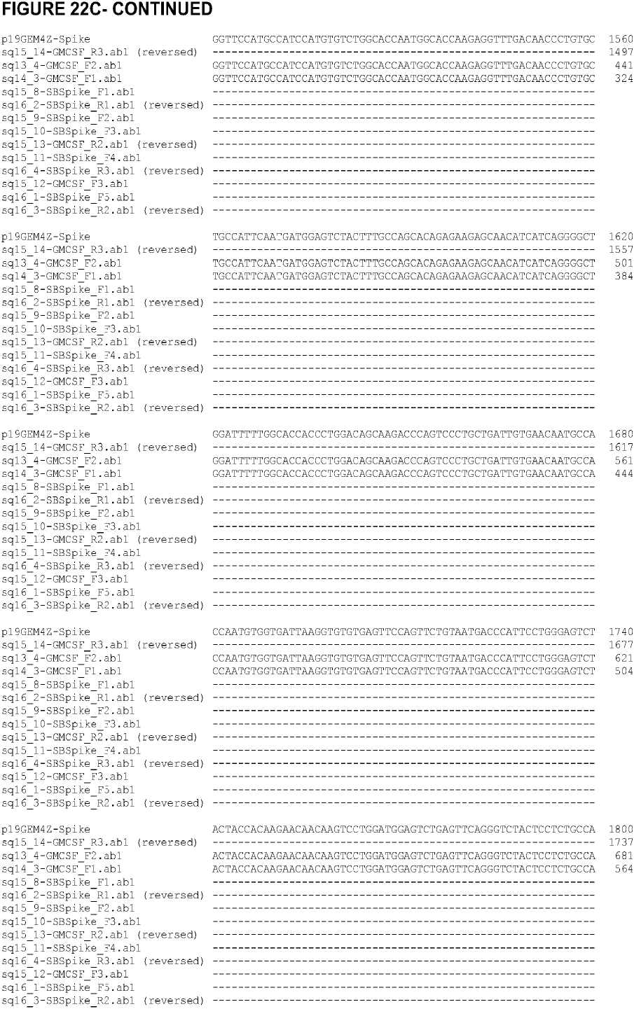

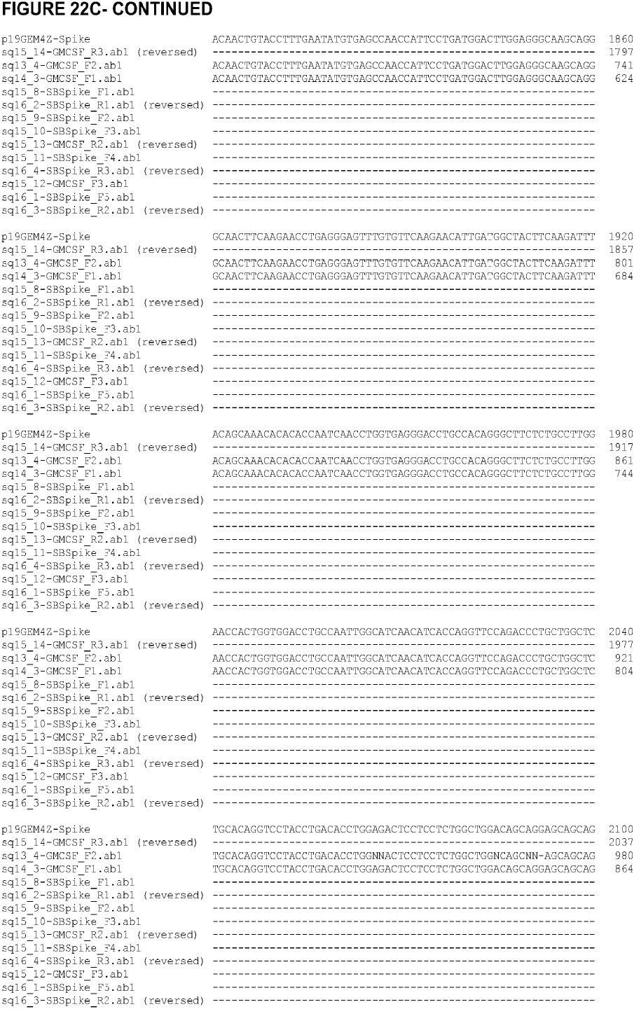

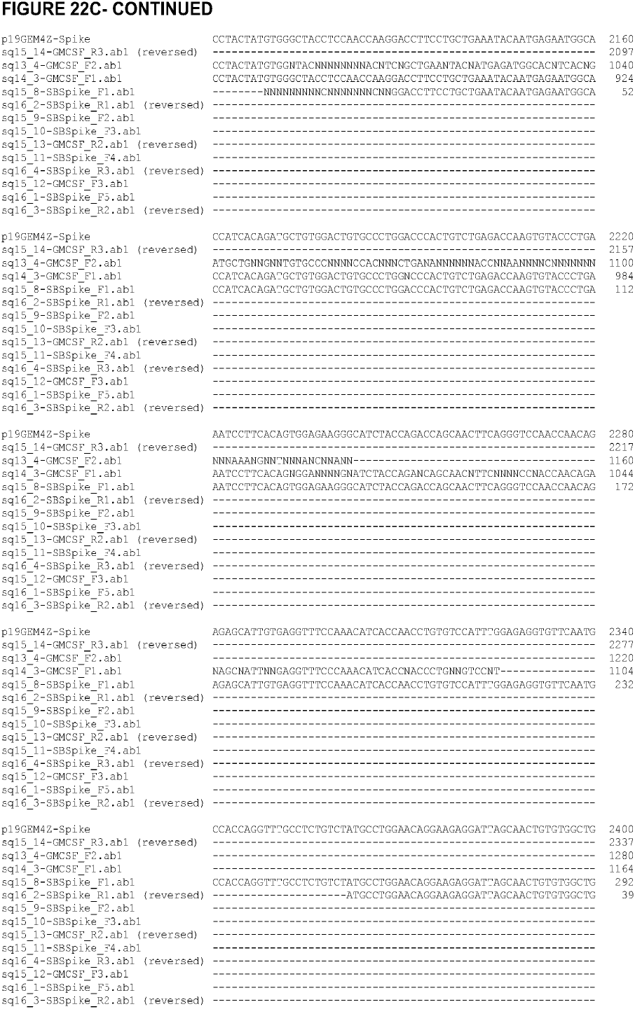

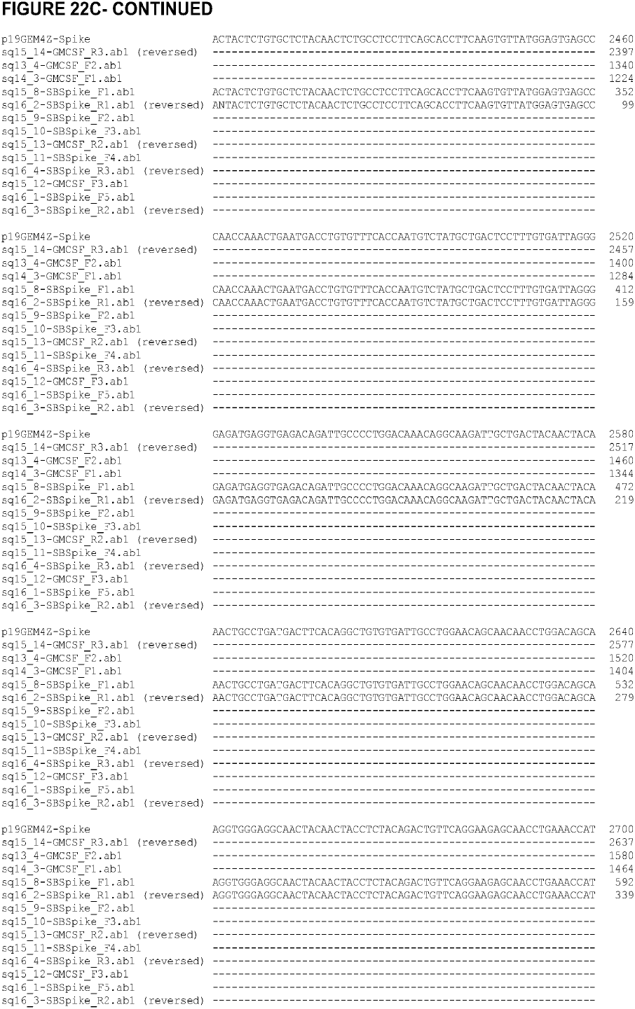

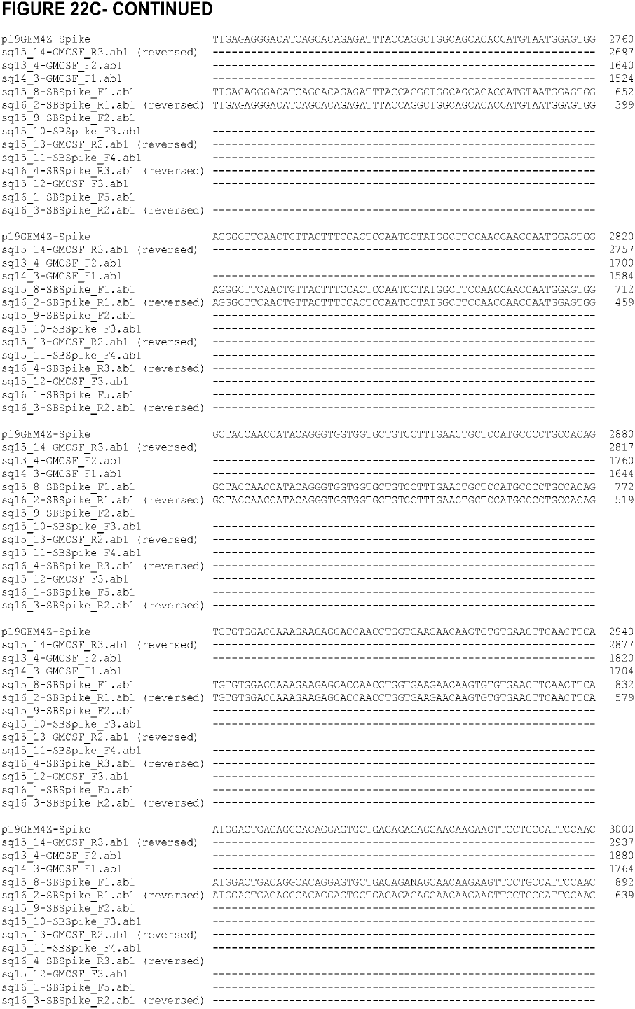

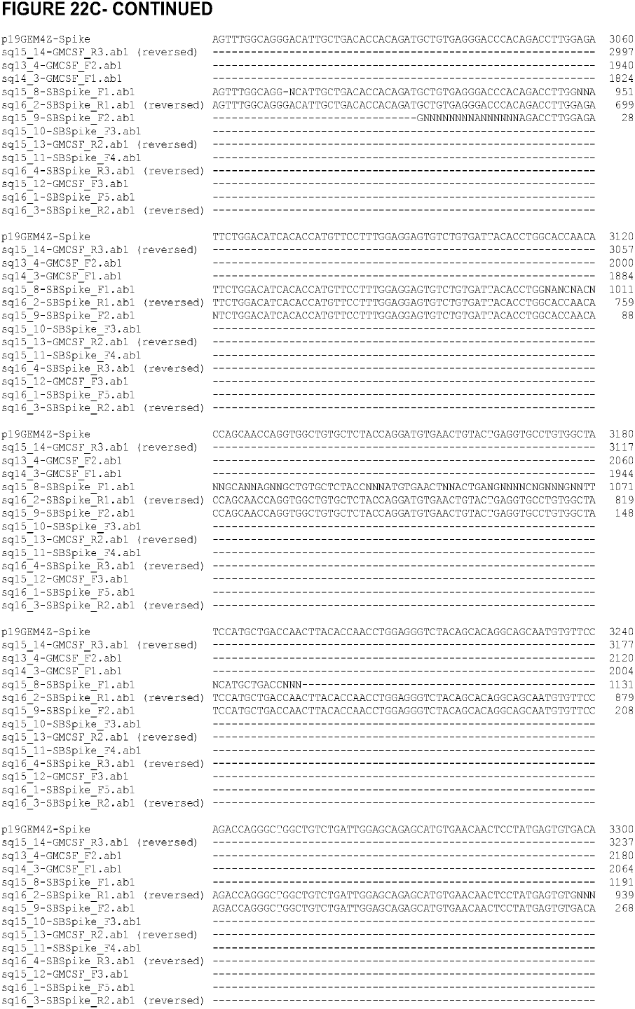

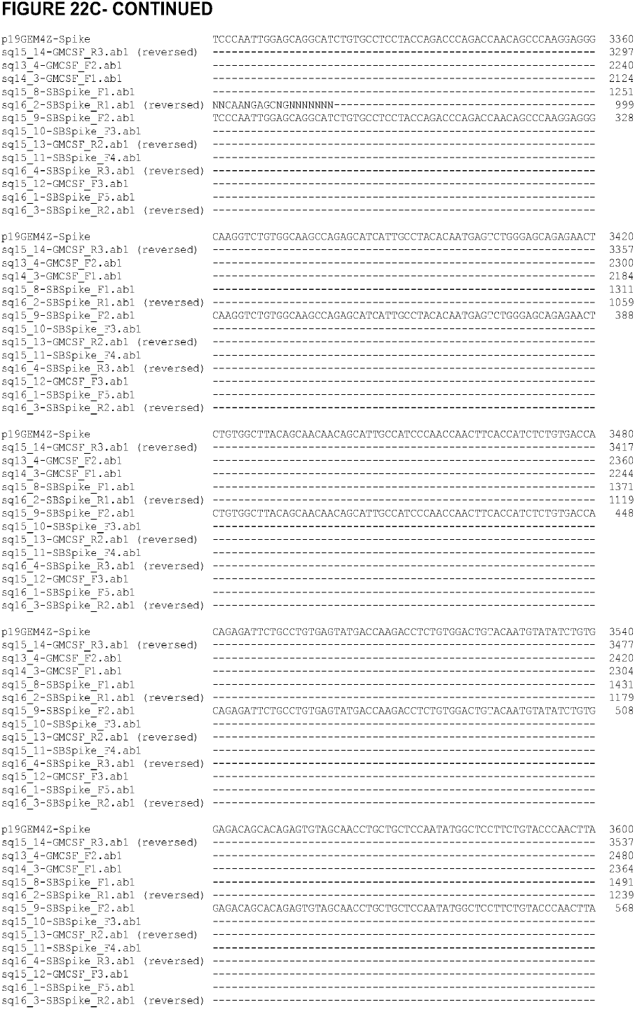

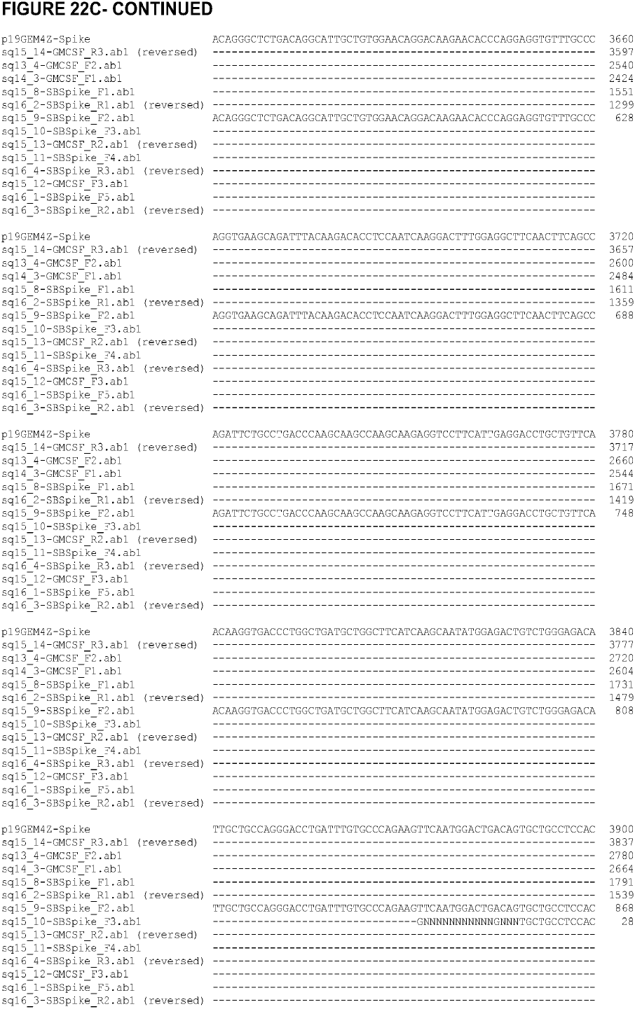

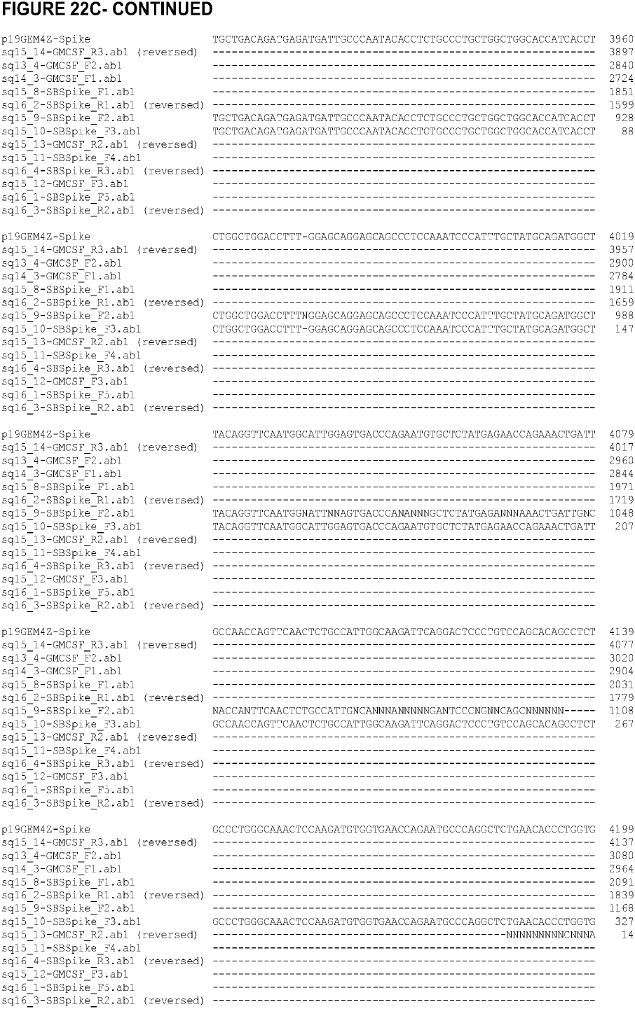

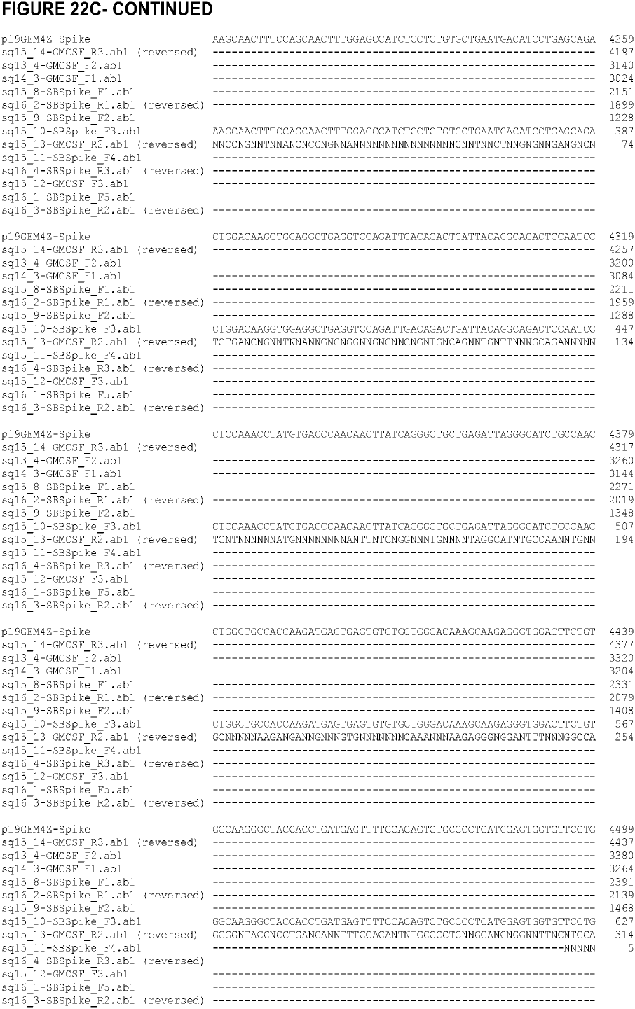

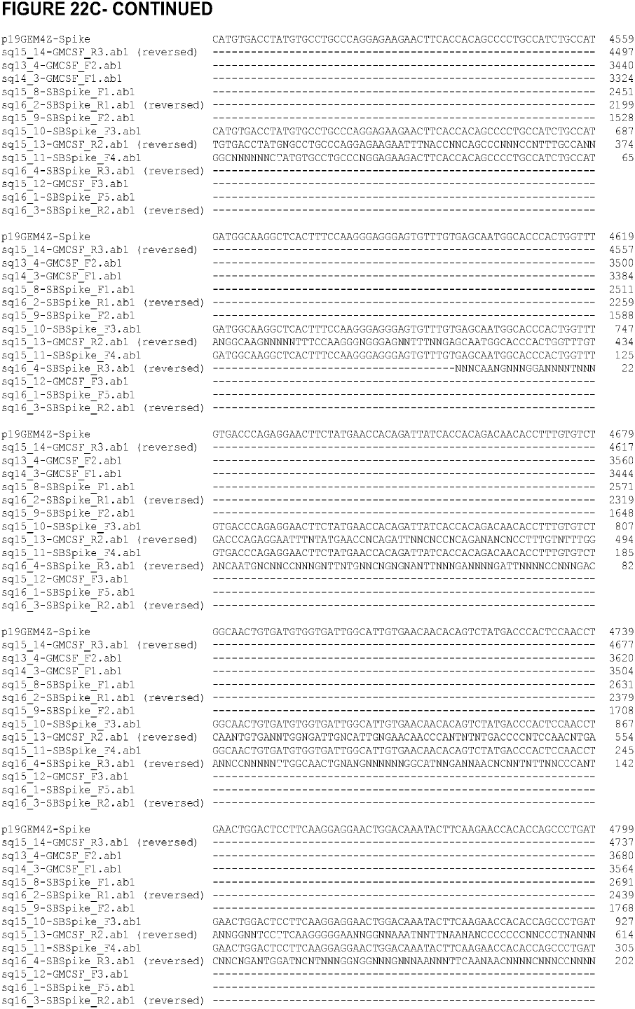

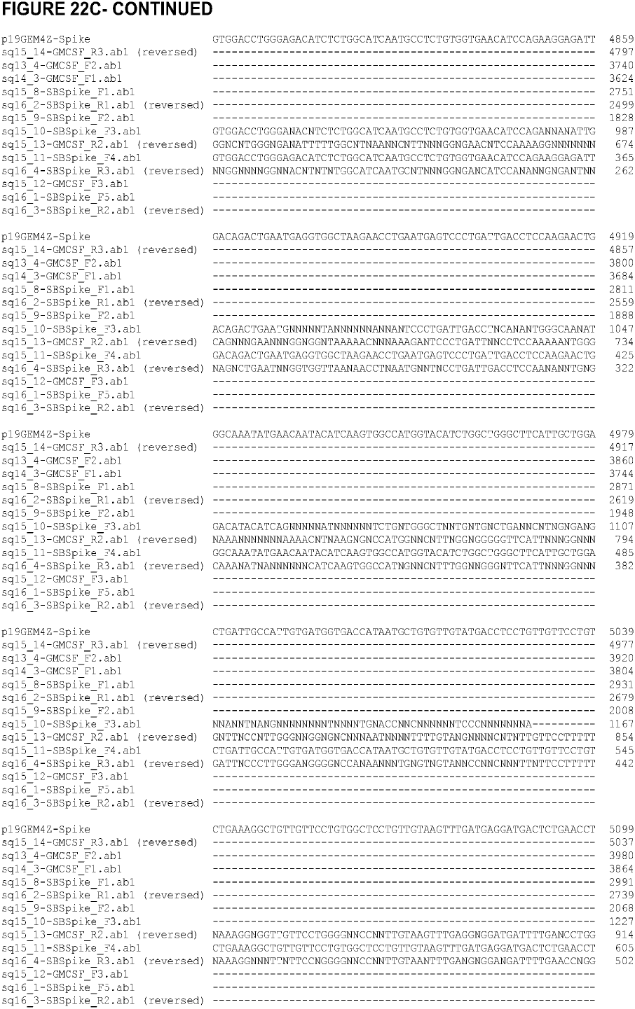

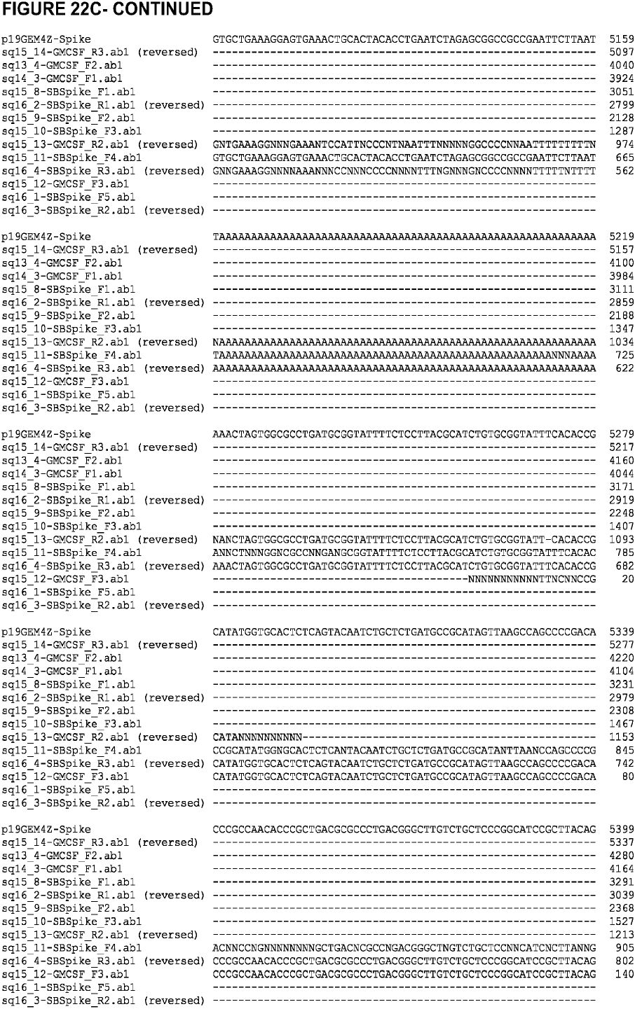

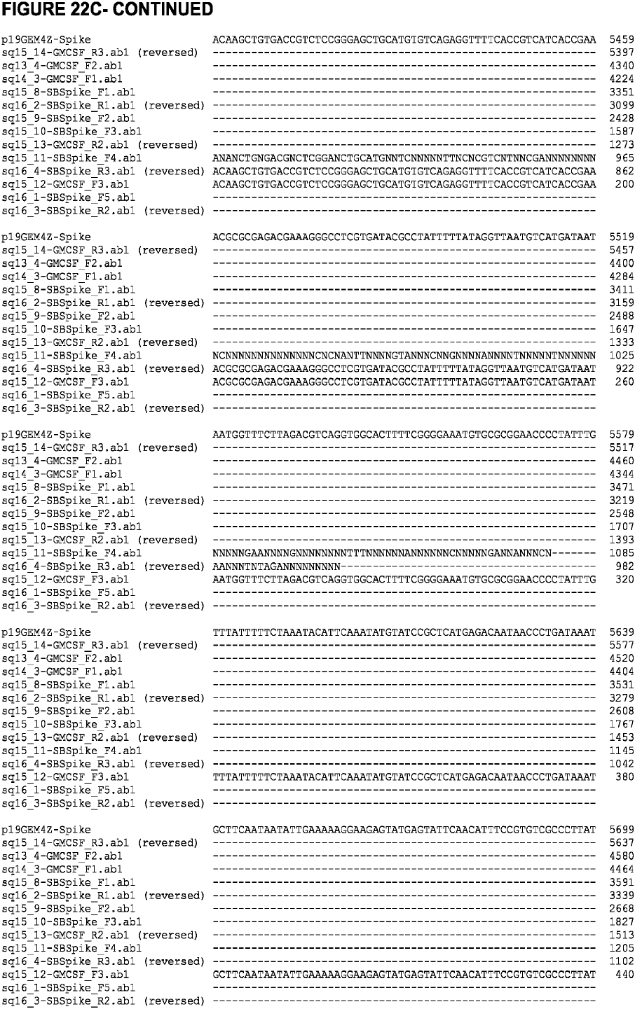

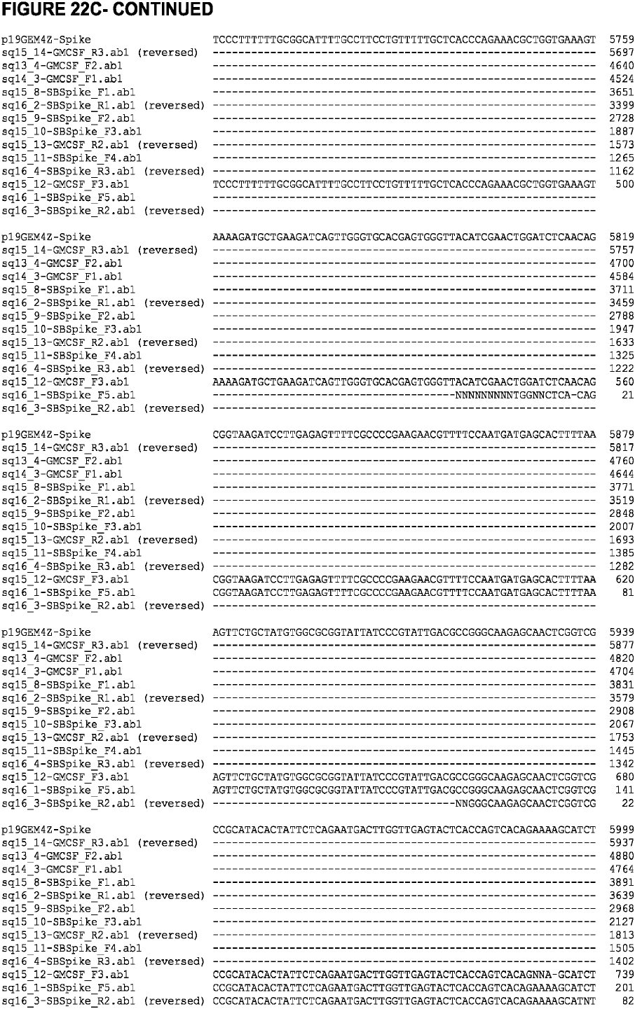

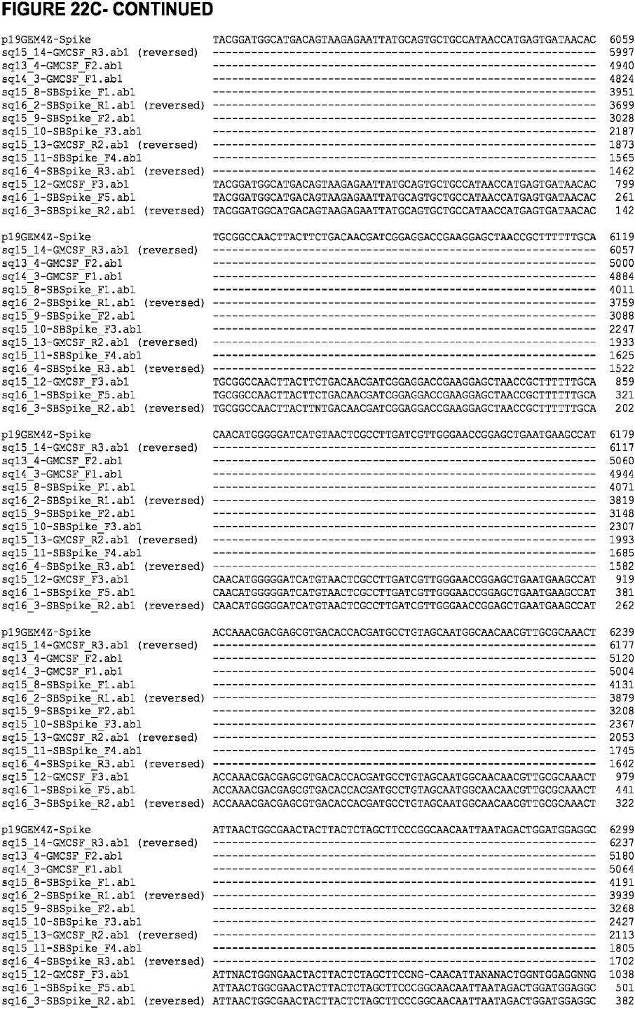

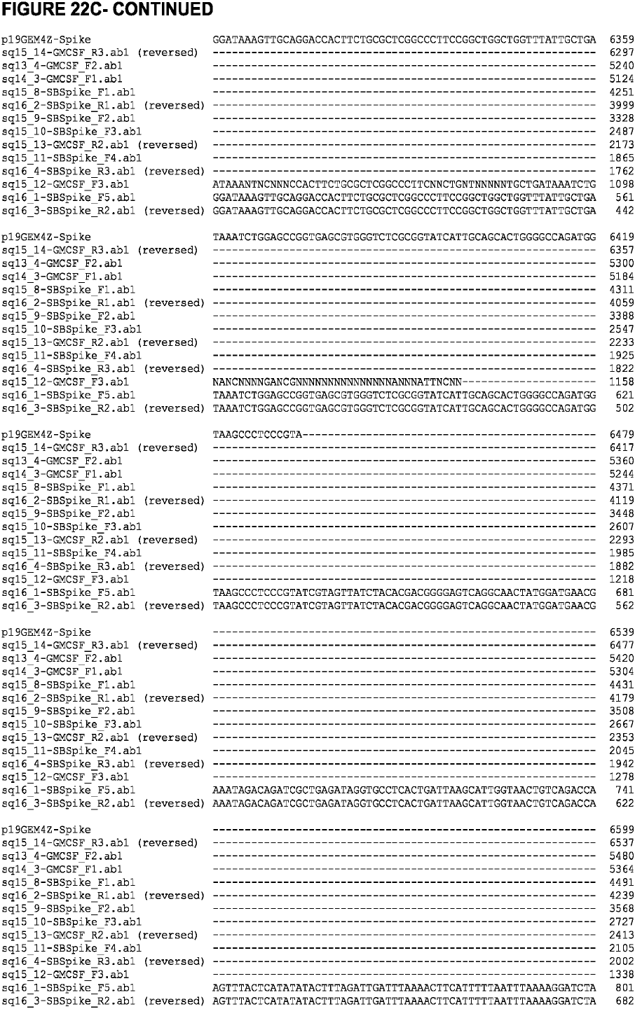

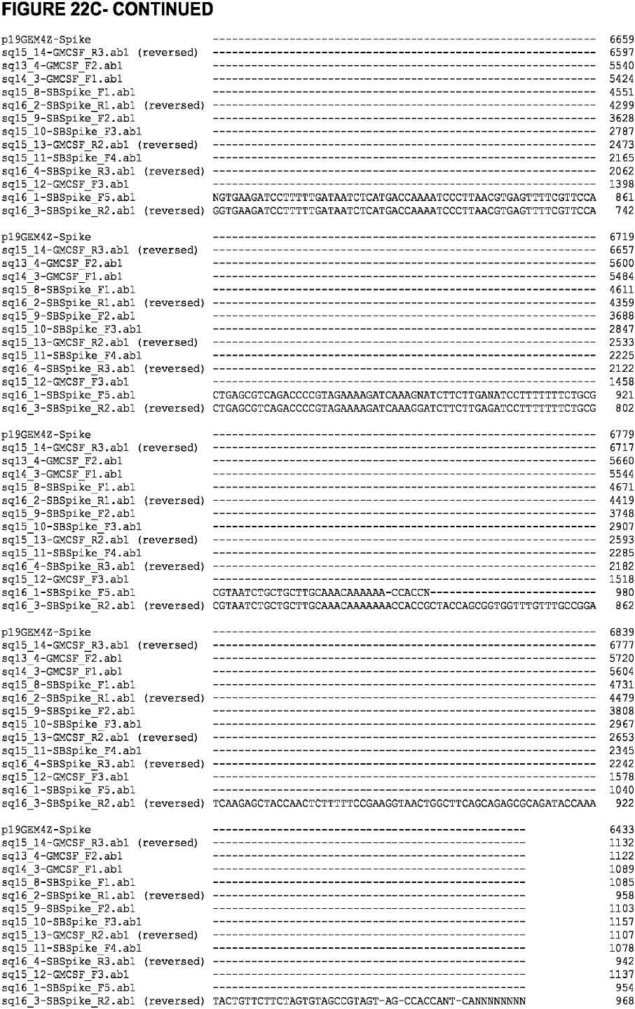

In various aspects, the viral protein encoded by the RNA (e.g., mRNA) of the presently disclosed nanoparticles is a full-length spike (S) protein of SARS-CoV-2. In exemplary instances, the viral protein comprises the sequence of SEQ ID NO: 3 or a sequence which is at least 75% identical to SEQ ID NO: 3 (e.g., at least 80% identical to SEQ ID NO: 3, at least 85% identical to SEQ ID NO: 3, at least 90% identical to SEQ ID NO: 3, at least 95% identical to SEQ ID NO: 3, at least 98% identical to SEQ ID NO: 3, or more). In various aspects, the mRNA is a product of in vitro transcription of a cDNA sequence encoding the full-length spike (S) protein of SARS-CoV-2. In various aspects, the mRNA is a product of in vitro transcription of a cDNA sequence encoding the viral protein having the sequence of SEQ ID NO: 3 or a sequence which is at least 75% identical to SEQ ID NO: 3 (e.g., at least 80% identical to SEQ ID NO: 3, at least 85% identical to SEQ ID NO: 3, at least 90% identical to SEQ ID NO: 3, at least 95% identical to SEQ ID NO: 3, at least 98% identical to SEQ ID NO: 3, or more). In various aspects, the mRNA is a product of in vitro transcription of a cDNA sequence of pGEM4z-Spike shown in FIG. 22 or a sequence highly similar thereto (e.g., at least 90% identical thereto). In exemplary instances, the nanoparticles of the present disclosure comprise mRNA encoded by the cDNA sequence of pGEM4z-Spike shown in FIG. 22 or a sequence highly similar thereto. In various aspects, the nanoparticle comprises the sequences set forth in SEQ ID NOs: 23-31

In various instances, the RNA encoding the viral protein is fused to an RNA encoding a lysosome-associated membrane protein (LAMP). LAMPs are membrane proteins specific to lysosomes comprising homologous lysosome-luminal domains separated by a proline-rich hinge region, a transmembrane domain and a cytoplasmic domain. A review on LAMPs is provided at Schwake et al., Traffic (2013) koi.org/10.1111/tra.12056. In certain aspects, the LAMP protein is a LAMP1, LAMP 2, LAMP3, LAMP4, or LAMP5 protein. The sequences of such LAMP proteins are known in the art. For example, the mRNA sequence of the LAMP1 precursor is available as NCBI Accession No. NM_005561.4 and the amino acid sequence of LAMP1 precursor is available as NCBI Accession No. NP_005552.3. Also, for example, the mRNA sequence of the LAMP2 isoform C precursor is available as NCBI Accession No. NM_001122606.1 and the amino acid sequence of LAMP2 isoform C precursor is available as NCBI Accession No. NP_001116078.1. The mRNA sequence of the LAMP3 precursor is available as NCBI Accession No. NM_014398.4 and the amino acid sequence of LAMP3 precursor is available as NCBI Accession No. NP_055213.2. The disclosures of the aforementioned accession numbers are hereby incorporated by reference.

Nanoparticles are further described in, e.g., International Patent Application No. PCT/US20/42606, which is hereby incorporated by reference in its entirety and in particular with respect to the discussion of nanoparticles comprising a positively-charged surface and an interior comprising a core and at least two nucleic acid layers, wherein each nucleic acid layer is positioned between a cationic lipid bilayer, and methods of manufacture.

### Methods of Manufacture

The present disclosure also provides a method of making a nanoparticle comprising a positively-charged surface and an interior comprising (i) a core and (ii) at least two nucleic acid layers, wherein each nucleic acid layer is positioned between a cationic lipid bilayer, said method comprising: (A) mixing nucleic acid molecules and liposomes at a nucleic acid (e.g., RNA): liposome ratio described herein, e.g., a ratio of about 1 to about 5 to about 1 to about 25, such as about 1 to 5 to about 1 to about 20, optionally, about 1 to about 15, to obtain nucleic acid (e.g., RNA)-coated liposomes. The liposomes are made by a process of making liposomes comprising drying a lipid mixture comprising a cationic lipid and an organic solvent by evaporating the organic solvent under a vacuum; and (B) mixing the RNA-coated liposomes with a surplus amount of liposomes.

In exemplary aspects, the nanoparticle made by the presently disclosed method accords with the descriptions of the presently disclosed nanoparticles described herein. For example, the nanoparticle made by the presently disclosed methods has a zeta potential of about +40 mV to about +60 mV, optionally, about +45 mV to about +55 mV. Optionally, the zeta potential of the nanoparticle made by the presently disclosed methods is about +50 mV. In various aspects, the core of the nanoparticle made by the presently disclosed methods comprises less than about 0.5 wt% nucleic acid and/or the core comprises a cationic lipid bilayer and/or the outermost layer of the nanoparticle comprises a cationic lipid bilayer and/or the surface of the nanoparticle comprises a plurality of hydrophilic moieties of the cationic lipid of the cationic lipid bilayer.

In exemplary aspects, the lipid mixture comprises the cationic lipid and the organic solvent at a ratio of about 40 mg cationic lipid per mL organic solvent to about 60 mg cationic lipid per mL organic solvent, optionally, at a ratio of about 50 mg cationic lipid per mL organic solvent. In various instances, the process of making liposomes further comprises rehydrating the lipid mixture with a rehydration solution to form a rehydrated lipid mixture and then agitating, resting, and sizing the rehydrated lipid mixture. Optionally, sizing the rehydrated lipid mixture comprises sonicating, extruding and/or filtering the rehydrated lipid mixture.

A description of an exemplary method of making a nanoparticle comprising a positively-charged surface and an interior comprising (i) a core and (ii) at least two nucleic acid layers, wherein each nucleic acid layer is positioned between a cationic lipid bilayer is provided herein at Example 1. Any one or more of the steps described in Example 1 may be included in the presently disclosed method. For instance, in some embodiments, the method comprises one or more steps required for preparing the RNA prior to being complexed with the liposomes. In exemplary aspects, the method comprises downstream steps to prepare the nanoparticles for administration to a subject, e.g., a human. In exemplary instances, the method comprises formulating the NP for intravenous injection. The method comprises in various aspects adding one or more pharmaceutically acceptable carriers, diluents, or excipients, and optionally comprises packaging the resulting composition in a container, e.g., a vial, a syringe, a bag, an ampoule, and the like. The container in some aspects is a ready-to-use container and optionally is for single-use.

Further provided herein are nanoparticles made by the presently disclosed method of making a nanoparticle.

### Cells and Populations Thereof

Additionally provided herein is a cell (e.g., an isolated cell or an ex vivo cell) comprising (e.g., transfected with) a nanoparticle of the present disclosure. In exemplary aspects, the cell is any type of cell that can contain the presently disclosed nanoparticle. The cell in some aspects is a eukaryotic cell, e.g., plant, animal, fungi, or algae. In alternative aspects, the cell is a prokaryotic cell, e.g., bacteria or protozoa. In exemplary aspects, the cell is a cultured cell. In alternative aspects, the cell is a primary cell, i.e., isolated directly from an organism, e.g., a human. The cell may be an adherent cell or a suspended cell, i.e., a cell that grows in suspension. The cell in exemplar aspects is a mammalian cell. Most preferably, the cell is a human cell. The cell can be of any cell type, can originate from any type of tissue, and can be of any developmental stage. In exemplary aspects, the cell comprising the liposome is an antigen presenting cell (APC). As used herein, “antigen presenting cell” or “APC” refers to an immune cell that mediates the cellular immune response by processing and presenting antigens for recognition by certain T cells. In exemplary aspects, the APC is a dendritic cell, macrophage, Langerhans cell or a B cell. In exemplary aspects, the APC is a dendritic cell (DC). In exemplary aspects, when the cells are administered to a subject, e.g., a human, the cells are autologous to the subject. In exemplary aspects, when the cells are administered to a subject, e.g., a human, the cells are allogeneic to the subject.

In exemplary instances, the immune cell is a tumor associated macrophage (TAM).

Also provided by the present disclosure is a population of cells wherein at least 50%, at least 60%, at least 70%, at least 80%, at least 90%, or at least 95% of the population are cells comprising (e.g., transfected with) a nanoparticle of the present disclosure. The population of cells in some aspects is heterogeneous cell population or, alternatively, in some aspects, is a substantially homogeneous population, in which the population comprises mainly cells comprising a nanoparticle of the present disclosure.

### Pharmaceutical Compositions

Provided herein are compositions comprising a nanoparticle of the present disclosure, a cell comprising the nanoparticle of the present disclosure, a population of cells of the present disclosure, or any combination thereof, and a pharmaceutically acceptable carrier, excipient or diluent. In exemplary aspects, the composition is a pharmaceutical composition comprising a plurality of nanoparticles according to the present disclosure and a pharmaceutically acceptable carrier, diluent, or excipient and intended for administration to a human. In exemplary aspects, the composition is a sterile composition. In exemplary instances, the composition comprises a plurality of nanoparticles of the present disclosure. Optionally, at least 50% of the nanoparticles of the plurality have a diameter between about 100 nm to about 250 nm. In various aspects, the composition comprises about 10¹⁰ nanoparticles per mL to about 10¹⁵ nanoparticles per mL, optionally about 10¹² nanoparticles ± 10% per mL.

In exemplary aspects, the composition of the present disclosure may comprise additional components other than the nanoparticle, cell comprising the nanoparticle, or population of cells. The composition, in various aspects, comprises any pharmaceutically acceptable ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, UK, 2000), which is incorporated by reference in its entirety. Remington’s Pharmaceutical Sciences, Sixteenth Edition, E. W. Martin (Mack Publishing Co., Easton, Pa., 1980), which is incorporated by reference in its entirety.

The composition of the present disclosure can be suitable for administration by any acceptable route, including parenteral and subcutaneous routes. Other routes include intravenous, intradermal, intramuscular, intraperitoneal, intranodal and intrasplenic, for example. In exemplary aspects, when the composition comprises the liposomes (not cells comprising the liposomes), the composition is suitable for systemic (e.g., intravenous) administration.

If the composition is in a form intended for administration to a subject, it can be made to be isotonic with the intended site of administration. For example, if the solution is in a form intended for administration parenterally, it can be isotonic with blood. The composition typically is sterile. In certain embodiments, this may be accomplished by filtration through sterile filtration membranes. In certain embodiments, parenteral compositions generally are placed into a container having a sterile access port, for example, an intravenous solution bag, or vial having a stopper pierceable by a hypodermic injection needle, or a prefilled syringe. In certain embodiments, the composition may be stored either in a ready-to-use form or in a form (e.g., lyophilized) that is reconstituted or diluted prior to administration.

### Use

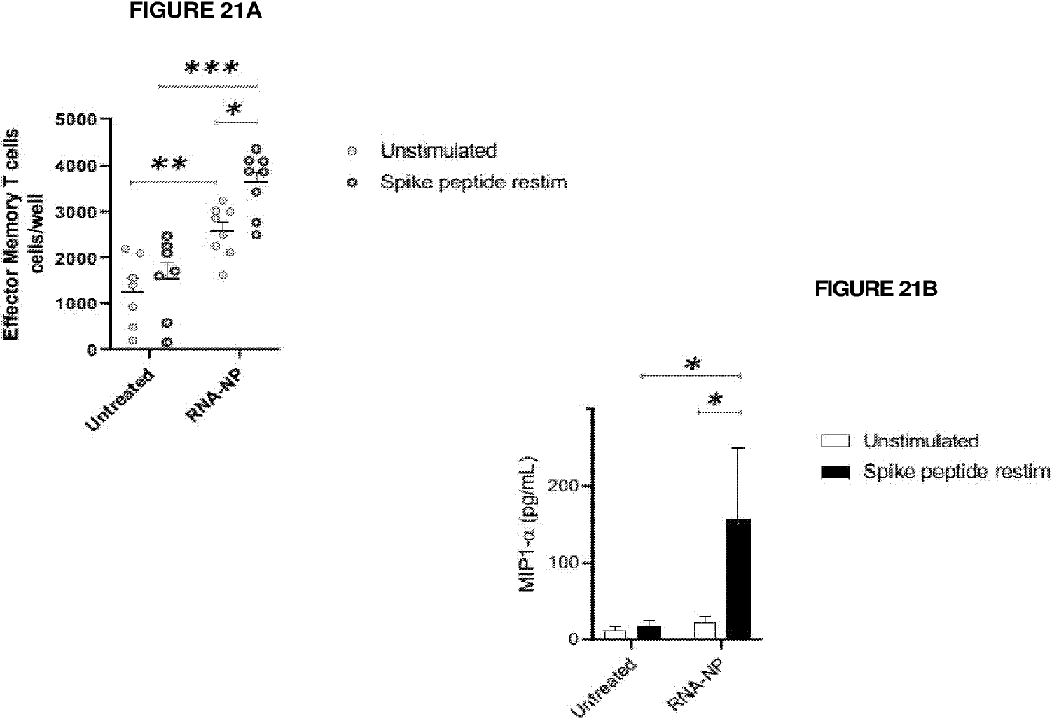

Without being bound to any particular theory, the data provided herein for the first time support the use of the presently disclosed ML RNA NPs for inducing or increasing an immune response against a SARS-Cov-2 virus in a subject. As shown in FIG. 21, the number of effector memory T cells specific to SARS-CoV-2 spike protein increase in mice vaccinated with a multilamellar RNA nanoparticle (ML RNA NP) comprising RNA encoding the SARS-CoV-2 spike protein, compared to the number of memory T cells specific to SARS-CoV-2 spike protein increase in mice not treated with the ML RNA NPs. Accordingly, a method of inducing or increasing an immune response against a SARS-CoV-2 virus in a subject is provided by the present disclosure. In exemplary embodiments, the method comprises administering to the subject the pharmaceutical composition of the present disclosure. In exemplary aspects, the nucleic acid molecules are mRNA. Optionally, the composition is systemically administered to the subject. For example, the composition is administered intravenously. In various aspects, the pharmaceutical composition is administered in an amount which is effective to activate dendritic cells (DCs) in the subject. In various instances, the immune response is a B-cell-mediated immune response, e.g., involving the production of antibodies, e.g., neutralizing antibodies. In various instances, the immune response involves an increase in the production of antibodies specific to a SARS-CoV-2 protein, optionally, IgM and/or IgG antibodies. In various instances, the immune response is a T cell-mediated immune response, for instance, a CD8+ T cell-mediated immune response. Optionally, the T cell-mediated immune response comprises an increase in effector memory T cells specific to SARS CoV-2. In various aspects, the T-cell mediated immune response comprises an increase in CD3+CD8+CD44+cells. In exemplary aspects, the immune response is the innate immune response. Optionally, the immune response is an innate immune response involving one or more of granulocytes, monocytes, macrophages, and natural kill (NK) cells. In various aspects, the presently disclosed multilamellar RNA NPs are administered to a subject for inducing or increasing an immune response against a SARS-Cov-2 virus in a subject, wherein DCs are activated, B-cells produce neutralizing antibodies to SARS-CoV-2, the number of effector memory T-cells specific to SARS CoV-2 is increased, the innate immune response against SARS-CoV-2 is activated, or a combination thereof. Optionally, the presently disclosed multilamellar RNA NPs are administered to a subject for increasing IFN-α levels or other type I interferons.

Also the data provided herein support the use of the presently disclosed RNA NPs for increasing dendritic cell (DC) activation in a subject. A method of activating DCs or increasing DC activation in a subject is accordingly furthermore provided. In exemplary embodiments, the method comprises administering to the subject the pharmaceutical composition of the present disclosure. In exemplary aspects, the nucleic acid molecules are mRNA. Optionally, the composition is systemically administered to the subject. For example, the composition is administered intravenously. In various aspects, the pharmaceutical composition is administered in an amount which is effective to increase an immune response against a tumor in the subject. In various instances, the immune response is a T cell-mediated immune response. In exemplary aspects, the immune response is the innate immune response.

Optionally, in various aspects of the disclosure, the T cell-mediated immune response comprises activity by tumor infiltrating lymphocytes (TILs).

As used herein, the term “increase” and words stemming therefrom may not be a 100% or complete increase. Rather, there are varying degrees of increasing of which one of ordinary skill in the art recognizes as having a potential benefit or therapeutic effect. In exemplary embodiments, the increase provided by the methods is at least or about a 10% increase (e.g., at least or about a 20% increase, at least or about a 30% increase, at least or about a 40% increase, at least or about a 50% increase, at least or about a 60% increase, at least or about a 70% increase, at least or about a 80% increase, at least or about a 90% increase, at least or about a 95% increase, or at least or about a 98% increase). In various aspects, the “increase” is in reference to baseline measurements (e.g., baseline immunity, sensitivity, or activation) in the absence of (e.g., prior to) administering the nanoparticles of the instant disclosure.

The present disclosure also provides a method of delivering RNA molecules to a lung of a subject. In exemplary embodiments, the method comprises administering to the subject a presently disclosed pharmaceutical composition. Provided herein are methods of delivery RNA to cells of a lung, comprising systemically (e.g., intravenously) administering a presently disclosed composition, wherein the composition comprises the nanoparticles. Also provided herein are methods of delivering RNA to cells in a lung. In exemplary embodiments, the method comprises systemically (e.g., intravenously) administering a presently disclosed composition, wherein the composition comprises the nanoparticle. The present disclosure also provides methods of activating antigen-presenting cells in a lung. In exemplary embodiments, the method comprises systemically (e.g., intravenously) administering a presently disclosed composition, wherein the composition comprises the NP. In various embodiments, the nanoparticles are administered to the subject by inhalation or intranasal administration.

In exemplary embodiments, the method described herein comprises administering a composition of the present disclosure in an amount effective to treat a disease or disorder in the subject or achieve a desired biological effect (e.g., an immune response against SARS-CoV-2). For example, in various aspects, RNA-NPs are administered in an amount which is effective to activate dendritic cells (DCs). One means of describing a dose of therapeutic is in terms of patient weight or mass (typically expressed as mg/kg). In various aspects, the amount of nanoparticles administered is sufficient to administer about 0.00050 mg/kg to about 1.5 mg/kg of nucleic acid (e.g., RNA, such as mRNA) to the subject (i.e., a dose of the pharmaceutical composition delivers about 0.00050 mg/kg to about 1.5 mg/kg of nucleic acid to the subject). The nucleic acid may be encapsulated into any suitable amount of liposome to administer the nucleic acid to the subject. As explained above, in various embodiments, the nucleic acid molecules may be present in the nanoparticle at a nucleic acid molecule: cationic lipid ratio of about 1 to about 5 to about 1 to about 25, such as about 1 to about 5 to about 1 to about 20, optionally, about 1 to about 18, about 1 to about 17, about 1 to about 15, about 1 to about 10, or about 1 to about 7.5. In various aspects, the amount of nucleic acid for administration is encapsulated into about 0.008 mg/kg to about 1.5 mg/kg of liposome material (“LP”). In various aspects, a dose of pharmaceutical composition comprises about 0.000625 mg/kg to about 0.08 mg/kg nucleic acid (e.g., RNA, such as mRNA), which is optionally encapsulated in about 0.009375 mg/kg to about 1.2 mg/kg LPs. Examples of doses suitable for use in the context of the disclosure include, but are not limited to about 0.000625 mg/kg of nucleic acid (e.g., mRNA) optionally encapsulated in about 0.009375 mg/kg LPs, about 0.00125 mg/kg nucleic acid (e.g., mRNA) optionally encapsulated in about 0.01875 mg/kg LPs, about 0.0025 mg/kg nucleic acid (e.g., mRNA) optionally encapsulated in about 0.0375 mg/kg LPs, about 0.005 mg/kg nucleic acid (e.g., mRNA) optionally encapsulated in about 0.075 mg/kg LPs, about 0.01 mg/kg nucleic acid (e.g., mRNA) optionally encapsulated in 0.15 mg/kg LPs, about 0.02 mg/kg nucleic acid (e.g., mRNA) optionally encapsulated in about 0.3 mg/kg LPs, about 0.04 mg/kg nucleic acid (e.g., mRNA) optionally encapsulated in about 0.6 mg/kg LPs, and about 0.08 mg/kg nucleic acid (e.g., mRNA) optionally encapsulated in 1.2 mg/kg LPs.

The nanoparticles or composition may be administered according to any treatment regimen suitable for a particular embodiment. It may be advantageous to administer multiple doses of the nanoparticles or composition, spacing out the administration of doses, depending on the treatment regimen selected for a particular patient. The nanoparticles or composition can be administered periodically over a time period of about 18 months or less, about 16 months or less, about 14 months or less, or about one year (12 months) or less (e.g., about nine months or less, about six months or less, or about three months or less). Examples of administration regimens include administering a dose, for example, daily (one time per day, two times per day, three times per day, four times per day, five times per day, six times per day), three times a week, twice a week, every two days, every three days, every four days, every five days, every six days, weekly (i.e., once a week), every two weeks, every three weeks, monthly, or bi-monthly. In various aspects, a dose of the nanoparticles or composition is/are administered to the subject once a week. In various aspects, a dose of the nanoparticles or composition is/are administered to the subject about every two weeks (i.e., about every 14 days). In various aspects, a dose of nanoparticles or composition is/are administered to the subject about once a month (i.e., about every 30 days).

In various aspects, the treatment regimen includes more frequent administration of nanoparticles or composition over an initial treatment period, then subsequent administrations spaced out over longer periods of time. For example, doses may be administered for an initial treatment period of about two weeks, about four weeks, about six weeks, about eight weeks, about 10 weeks, or about 12 weeks, during which multiple doses are administered at an interval of, e.g., once a week or every two weeks. The treatment regimen may then comprise a subsequent treatment period wherein multiple doses are administered with longer intervals between doses, e.g., every two weeks, every three weeks, every four weeks (i.e. once a month), every five weeks, every six weeks, and the like. The subsequent treatment period may comprise about four weeks, about five weeks, about six weeks, about seven weeks, about eight weeks, about three months, about four months, about five months, about six months, about seven months, about eight months, about nine months, about 10 months, about 11 months, about 12 months, or more. The length of the initial treatment period and the subsequent treatment period may, or may not, be the same (the interval between administrations within the initial treatment period and subsequent treatment period will differ). In an exemplary aspect of the disclosure, a dose of nanoparticles or composition is administered about every two weeks for an initial treatment period of about four weeks (totaling three initial doses), which is followed by administration of a dose of nanoparticles or composition about once a month for a period of about twelve months.

The present disclosure provides methods of delivering RNA molecules to cells. In exemplary embodiments, the method comprises incubating the cells with the NPs of the present disclosure. In exemplary instances, the cells are antigen-presenting cells (APCs), optionally, dendritic cells (DCs). In various instances, the APCs (e.g., DCs) are obtained from a subject. In certain aspects, the RNA molecules encode a SARS-CoV-2 protein, e.g., a spike protein, membrane protein, envelope protein, or nucleocapsid protein, or a combination thereof. Indeed, in any aspect of the disclosure, the NPs may comprise a population of different RNA molecules that encode different target antigens.

Once RNA has been delivered to the cells, the cells may be administered to a subject for treatment of a disease (e.g., an infection). Accordingly, the present disclosure provides a method of treating a subject with a disease. In exemplary embodiments, the method comprises delivering RNA molecules to cells of the subject in accordance with the above-described method of delivering RNA molecules to cells. In some aspects, RNA molecules are delivered to the cells ex vivo and the cells are administered to the subject. Alternatively, the method comprises administering the liposomes directly to the subject. In exemplary embodiments, the method of treating a subject with a disease comprises administering a composition of the present disclosure in an amount effective to treat the disease in the subject. In exemplary aspects, the disease is COVID-19. In exemplary aspects, the composition comprises the liposomes, and optionally, the composition comprising the liposomes are intravenously administered to the subject or administered by inhalation or intranasal administration. In alternative aspects, the composition comprises cells transfected with the liposome. Optionally, the cells of the composition are APCs, optionally, DCs. In exemplary instances, the DCs are isolated from white blood cells (WBCs) obtained from the subject, optionally, wherein the WBCs are obtained via leukapheresis.

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating a disease of the present disclosure can provide any amount or any level of treatment. Furthermore, the treatment provided by the method may include treatment of one or more conditions or symptoms or signs of the disease being treated. For instance, the treatment method of the present disclosure may inhibit or alleviate (in whole or in part) one or more symptoms of the disease. Examples of symptoms of COVID-19 include, e.g., fever or chills, cough, shortness of breath or difficulty breathing, fatigue, body aches, headache, loss of taste or smell, sore throat, congestion or runny nose, nausea, vomiting, and diarrhea. “Treatment” also includes reduction in viral load. Also, the treatment provided by the methods of the present disclosure may encompass slowing the progression of the disease.

The term “treat” also encompasses prophylactic treatment of the disease. Accordingly, the treatment provided by the presently disclosed method may delay the onset or reoccurrence/relapse of the disease being prophylactically treated. In exemplary aspects, the method delays the onset of the disease by 1 day, 2 days, 4 days, 6 days, 8 days, 10 days, 15 days, 30 days, two months, 4 months, 6 months, 1 year, 2 years, 4 years, or more. The prophylactic treatment encompasses reducing the risk of the disease (i.e., reducing the risk of productive infection of SARS-CoV-2). In exemplary aspects, the method reduces the risk of the disease 2-fold, 5-fold, 10-fold, 20-fold, 50-fold, 100-fold, or more.

In certain aspects, the method of treating the disease may be regarded as a method of inhibiting the disease, or a symptom thereof. As used herein, the term “inhibit” and words stemming therefrom may not be a 100% or complete inhibition or abrogation. Rather, there are varying degrees of inhibition of which one of ordinary skill in the art recognizes as having a potential benefit or therapeutic effect. The presently disclosed methods may inhibit the onset or re-occurrence of the disease or a symptom thereof to any amount or level. In exemplary embodiments, the inhibition provided by the methods is at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about a 80% inhibition, at least or about a 90% inhibition, at least or about a 95% inhibition, at least or about a 98% inhibition).

With regard to the foregoing methods, the NPs or the composition comprising the same in some aspects is systemically administered to the subject. Optionally, the method comprises administration of the liposomes or composition by way of parenteral administration. In various instances, the liposome or composition is administered to the subject intravenously.

### Subjects

The subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human. In some aspects, the human is an adult aged 18 years or older. In some aspects, the human is a child aged 17 years or less.

In various aspects of the methods described herein relating to cancer, the subject is a high-risk brain cancer patient.

In various aspects, the subject is a human subject diagnosed with COVID-19 or at risk of exposure to SARS-CoV-2. In various aspects, the composition is provided to the subject in an amount effective to reduce the risk of infection with SARS-CoV-2, to reduce or minimize virus titer, to reduce the time of infection, and/or to reduce the severity of symptoms of COVID-19.

The following examples are given merely to illustrate the present invention and not in any way to limit its scope.

### EXAMPLES

### Example 1

This example describes a method of making nanoparticles of the present disclosure.

### Preparation of DOTAP Liposomes

On Day 1, the following steps were carried out in the fume hood. Water was added to a rotavapor bath. Chloroform (20 mL) was poured into a sterile, glass graduated cylinder. After opening a vial containing 1 g of DOTAP, 5 mL chloroform was added to the DOTAP vial using a glass pipette. The volume of chloroform and DOTAP was then transferred into a 1-L evaporating flask. The DOTAP vial was washed by adding a second 5-mL volume of chloroform to the DOTAP vial to dissolve any remaining DOTAP in the vial and then transferring this volume of chloroform from the DOTAP vial to the evaporating flask. This washing step was repeated 2 more times until all the chloroform in the graduated cylinder was used. The evaporating flask was then placed into the Buchi rotavapor. The water bath was turned on and adjusted to 25° C. The evaporating flask was moved downward until it touched the water bath. The rotation speed of the rotavapor was adjusted to 2. The vacuum system was turned on and adjusted to 40 mbar. After 10 minutes, the vacuum system was turned off and the chloroform was collected from the collector flask. The amount of chloroform collected was measured. Once the collector flask is repositioned, the vacuum was turned on again and the contents in the evaporating flask was allowed to dry overnight until the chloroform was completely evaporated.

On Day 2, using a sterile graduated cylinder, PBS (200 mL) was added to a new, sterile 500-mL PBS bottle maintained at room temperature. A second 500-mL PBS bottle was prepared for collecting DOTAP. The Buchi rotavapor water bath was set to 50° C. PBS (50 mL) was added into the evaporating flask using a 25-mL disposable serological pipette. The evaporating flask was positioned in the Buchi rotavapor and moved downward until ⅓ of the flask was submerged into the water bath. The rotation speed of the rotavapor was set to 2, allowed to rotate for 10 min, and then rotation was turned off. A 50-mL volume of PBS with DOTAP from the evaporating flask was transferred to the second 500 mL PBS bottle. The steps were repeated (3-times) until the entire volume of PBS in the PBS bottle was used. The final volume of the second 500 mL PBS bottle was 400 mL. The lipid solution in the second 500 mL PBS bottle was vortexed for 30 s and then incubated at 50° C. for 1 hour. During the 1 hour incubation, the bottle was vortexed every 10 min. The second 500 mL PBS bottle was allowed to rest on overnight at room temperature.

On Day 3, PBS (200 mL) was added to the second 500 mL PBS bottle containing DOTAP and PBS. The second 500 mL PBS bottle was placed into an ultrasonic bath. Water was filled in the ultrasonic bath and the second 500 mL PBS bottle was sonicated for 5 min. The extruder was washed with PBS (100 mL) and this wash step was repeated. A 0.45 µm pore filter was assembled into a filtration unit and a new (third) 500 mL PBS bottle was positioned into the output tube of the extruder. In a biological safety cabinet, the DOTAP-PBS mixture was loaded into the extruder, until about 70% of the third PBS bottle was filled. The extruder was then turned on and the DOTAP PBS mixture was added until all the mixture was run through the extruder. Subsequently, a 0.22 µm pore filter was assembled into the filtration unit and a new (third) 500 mL PBS bottle was positioned into the output tube of the extruder. The previously filtered DOTAP-PBS mixture was loaded and run again throughout. The samples comprising DOTAP lipid nanoparticles (NPs) in PBS were then stored at 4° C.

### RNA Preparation

Prior to incorporation into NPs, RNA is prepared as follows:

### Preparation of SARS CoV-2-Specific mRNA

Plasmids, e.g., a modified psP73 vector (Promega) containing a T7 RNA polymerase promoter and a poly-A tail that is 64 nucleotides long (pSP73-Sph/A64) protecting RNA from degradation, were obtained and made to comprise cDNA encoding mRNA encoding the full length SARS CoV-2 Spike protein. The plasmids were linearized using restriction enzymes (i.e., Spel) and purified with Qiagen PCR MiniElute kits. The linearized DNA was subsequently in vitro transcribed using the mMESSAGE mMACHINE T7™ transcription kit (Invitrogen) per manufacturer’s protocol. The transcribed mRNA was then cleaned up using RNA Maxi kits (Qiagen).

In exemplary aspects, the mRNA encodes the S protein, e.g., comprising the amino acid sequence of SEQ ID NO: 3. In exemplary aspects, the cDNA encoding the Spike protein mRNA had the sequence as shown as the top line of the series of sequences shown in FIG. 22. In exemplary aspects, the mRNA encoding the S protein is mixed with mRNA encoding other proteins of SARS-CoV-2, e.g., such as those described herein or known in the art. In an exemplary aspect, the mRNA is as depicted in FIG. 19.

### Preparation of RNA for Examples 2-10

Prior to incorporation into NPs, RNA was prepared in one of a few ways (A) to (C). Total tumor RNA was prepared by isolating total RNA (including rRNA, tRNA, mRNA) from tumor cells. In vitro transcribed mRNA was prepared by carrying out in vitro transcription reactions using cDNA templates produced by reverse transcription of total tumor RNA. Tumor antigen-specific and non-specific RNAs were either made in-house or purchased from a vendor.

(A) Total Tumor RNA: Total tumor-derived RNA from tumor cells (e.g., B16F0, B16F10, and KR158-luc) is isolated using commercially available RNeasy mini kits (Qiagen) based on manufacturer instructions.

(B) In vitro Transcribed mRNA: Briefly, RNA is isolated using commercially available RNeasy mini kits (Qiagen) per manufacturer’s instructions and cDNA libraries were generated by RT-PCR. Using a SMARTScribe Reverse Transcriptase kit (Takara), a reverse transcriptase reaction by PCR was performed on the total tumor RNA in order to generate cDNA libraries. The resulting cDNA was then amplified using Takara Advantage 2 Polymerase mix with T7/SMART and CDS III primers, with the total number of amplification cycles determined by gel electrophoresis. Purification of the cDNA was performed using a Qiagen PCR purification kit per manufacturer’s instructions. In order to isolate sufficient mRNA for use in each RNA-nanoparticle vaccine, mMESAGE mMACHINE (Invitrogen) kits with T7 enzyme mix were used to perform overnight in vitro transcription on the cDNA libraries. Housekeeping genes were assessed to ensure fidelity of transcription. The resulting mRNA was then purified with a Qiagen RNeasy Maxi kit to obtain the final mRNA product.

### (C) Tumor Antigen-Specific and Non-Specific mRNA

Plasmids comprising DNA encoding tumor antigen-specific RNA (RNA encoding, e.g., pp65, OVA) and non-specific RNA (RNA encoding, e.g., Green Fluorescent Protein (GFP), luciferase) are linearized using restriction enzymes (i.e., Spel) and purified with Qiagen PCR MiniElute kits. Linearized DNA is subsequently transcribed using the mmRNA in vitro transcription kit (Life technologies, Invitrogen) and cleaned up using RNA Maxi kits (Qiagen). In alternative methods, non-specific RNA is purchased from Trilink Biotechnologies (San Diego, CA).

### Preparation of Multilamellar RNA Nanoparticles (NPs)

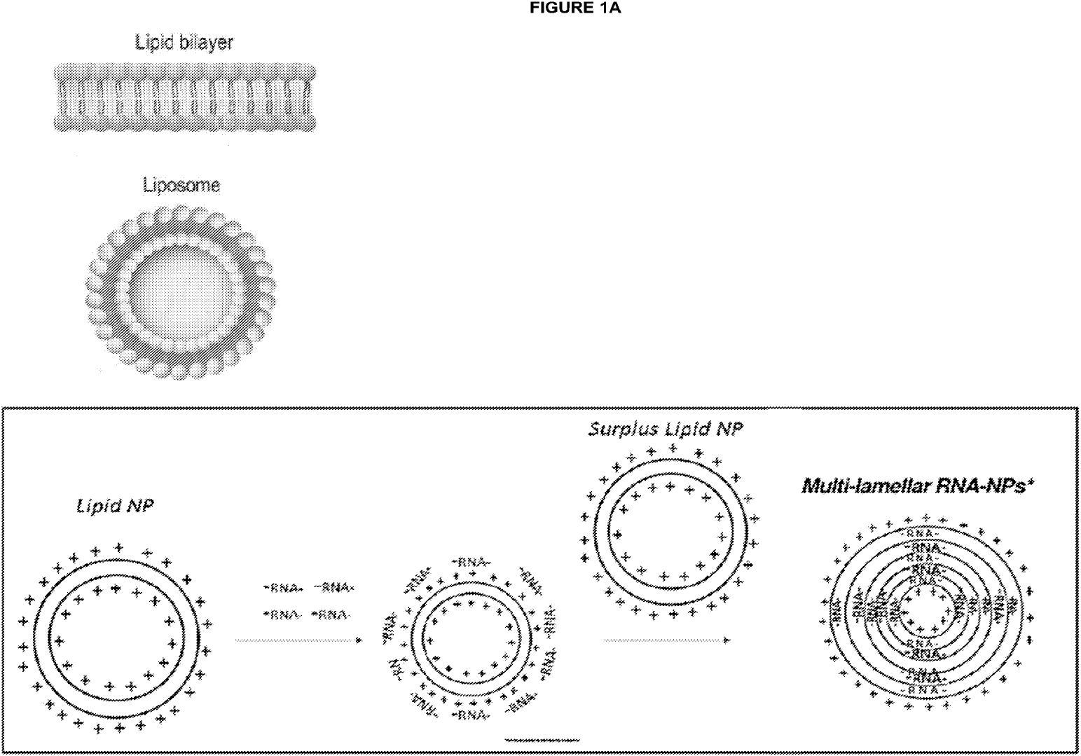

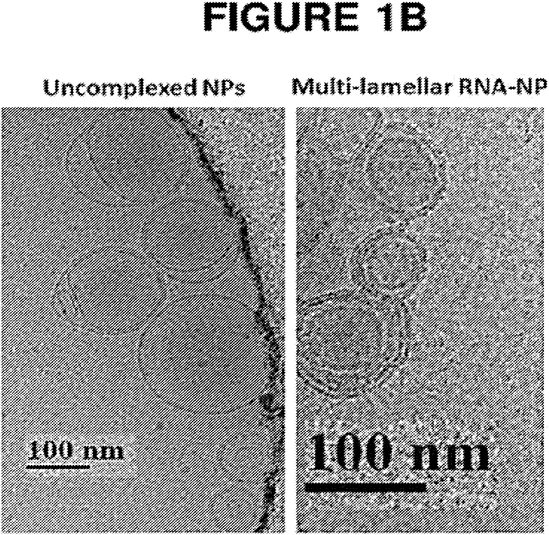

The DOTAP lipid NPs were complexed with RNA to make multilamellar RNA-NPs which were designed to have several layers of mRNA contained inside a tightly coiled liposome with a positively charged surface and an empty core (FIG. 1A). Briefly, in a safety cabinet, RNA was thawed from -80° C. and then placed on ice, and samples comprising PBS and DOTAP (e.g., DOTAP lipid NPs) were brought up to room temperature. Once components were prepared, the desired amount of RNA was mixed with PBS in a sterile tube. To the sterile tube containing the mixture of RNA and PBS, the appropriate amount of DOTAP lipid NPs was added without any physical mixing (without e.g., inversion of the tube, without vortexing, without agitation). The mixture of RNA, PBS, and DOTAP was incubated for about 15 minutes to allow multilamellar RNA-NP formation. After 15 min, the mixture was gently mixed by repeatedly inverting the tube. The mixture was then considered ready for systemic (i.e., intravenous) administration.

The amount of RNA and DOTAP lipid NPs (liposomes) used in the above preparation is pre-determined or pre-selected. In some instances, a ratio of about 15 µg liposomes per about 1 µg RNA were used. For instance, about 75 µg liposomes are used per ~5 µg RNA or about 375 µg liposomes are used per ~25 µg RNA. In other instances, about 7.5 µg liposomes were used per 1 µg RNA. Thus, in exemplary instances, about 1 µg to about 20 µg liposomes are used for every µg RNA used.

### Example 2

This example describes the characterization of the nanoparticles of the present disclosure.

### Cryo-Electron Microscopy (CEM)

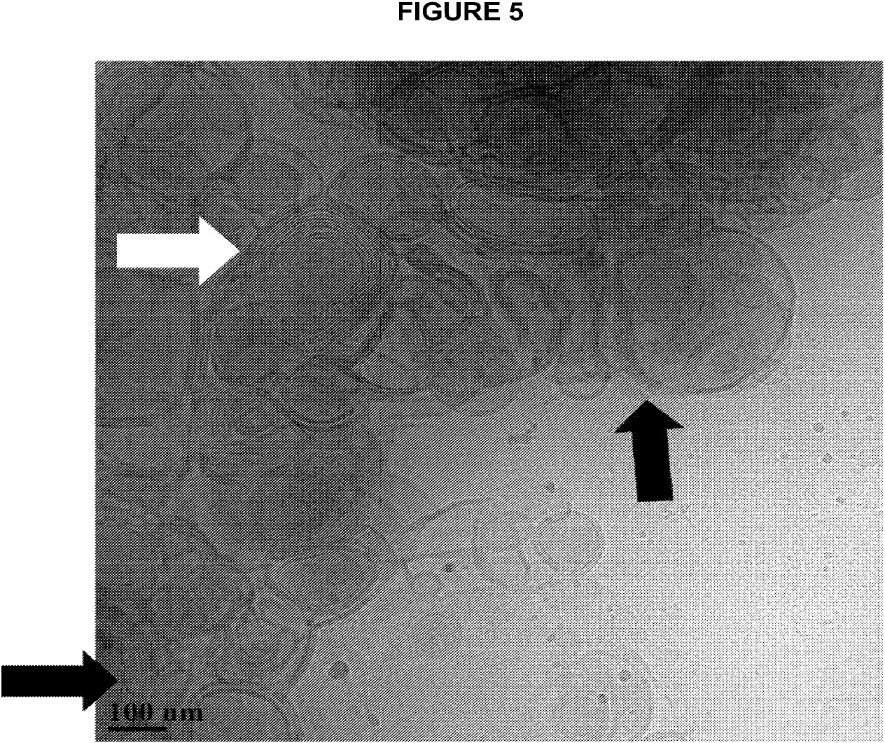

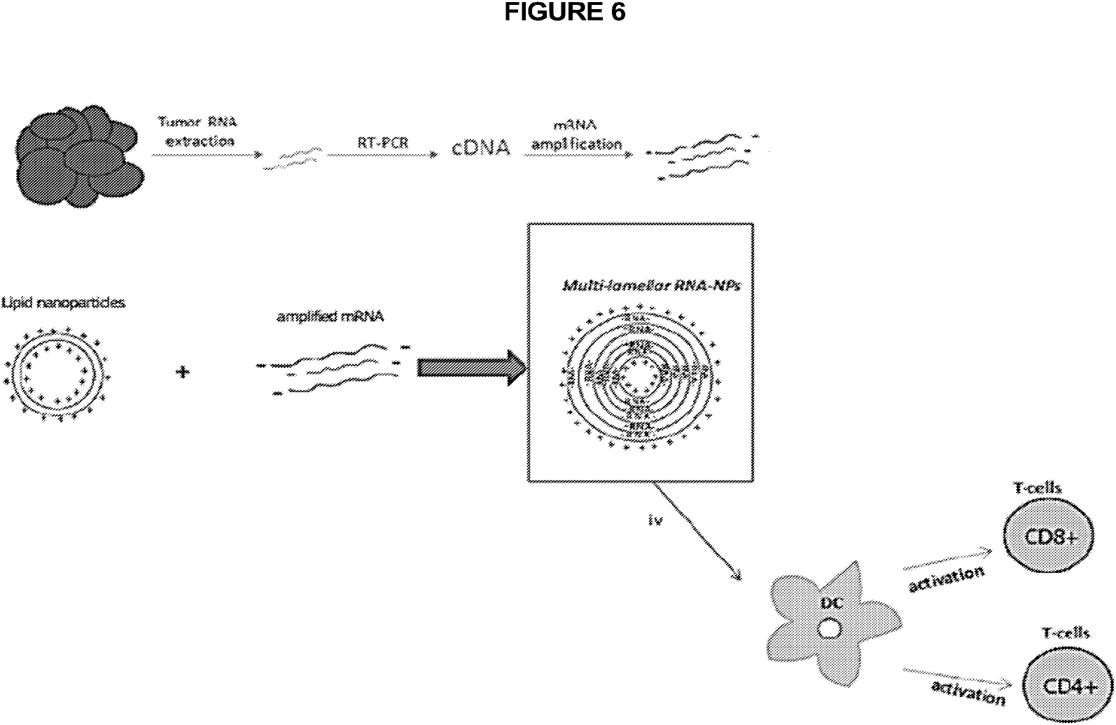

CEM was used to analyze the structure of multilamellar RNA-NPs prepared as described in Example 1 and control NPs devoid of RNA (uncomplexed NPs) which were made by following all the steps of Example 1, except for the steps under “RNA Preparation” and “Preparation of Multilamellar RNA nanoparticles (NPs)”. CEM was carried out as essentially described in Sayour et al., Nano Lett 17(3) 1326-1335 (2016). Briefly, samples comprising multilamellar RNA-NPs or control NPs were kept on ice prior to being loaded in a snap-freezed in Vitrobot (and automated plunge-freezer for cryoTEM, that freezes samples without ice crystal formation, by controlling temperature, relative humidity, blotting conditions and freezing velocity). Samples were then imaged in a Tecnai G2 F20 TWIN 200 kV / FEG transmission electron microscope with a Gatan UltraScan 4000 (4k x4k) CCD camera. The resulting CEM images are shown in FIG. 1B. The right panel is a CEM image of multilamellar RNA-NPs and the left panel is a CEM image of control NPs (uncomplexed NPs). As shown in FIG. 1B, the control NPs contained at most 2 layers, whereas multilamellar RNA NPs contained several layers. FIG. 5 provides another CEM image of exemplary multilamellar RNA NPs. Here, the multiple layers of RNA layers alternating with lipid layers are especially evident.

### Zeta Potentials

Zeta potentials of multilamellar RNA NPs were measured by phase analysis light scattering (PALS) using a Brookhaven ZetaPlus instrument (Brookhaven Instruments Corporation, Holtsville, NY), as essentially described in Sayour et al., Nano Lett 17(3) 1326-1335 (2016). Briefly, uncomplexed NPs or RNA-NPs (200 µL) were resuspended in PBS (1.2 mL) and loaded in the instrument. The samples were run at 5 runs per sample, 25 cycles each run, and using the Smoluchowski model.

The zeta potential of the multilamellar RNA NPs prepared as described in Example 1 was measured at about +50 mV. Interestingly, this zeta potential of the multilamellar RNA NPs was much higher than those described in Sayour et al., Oncoimmunology 6(1): e1256527 (2016), which measured at around +27 mV. Without being bound to any particular theory, the way in which the DOTAP lipid NPs are made for use in making the multilamellar RNA NPs (Example 1) involving a vacuum-seal method for evaporating off chloroform leads to less environmental oxidation of the DOTAP lipid NPs, which, in turn, may allow for a greater amount of RNA to complex with the DOTAP NPs and/or greater incorporation of RNA into the DOTAP lipid NPs.

### RNA Incorporation by Gel Electrophoresis

A gel electrophoresis experiment was conducted to measure the amount of RNA incorporated into ML liposomes. Based on this experiment, it was qualitatively shown that nearly all, if not all, of the RNA used in the procedure described in Example 1 was incorporated into the DOTAP lipid NPs. Additional experiments to characterize the extent of RNA incorporation are carried out by measuring RNA-NP density and comparing this parameter to that of lipoplexes.

### Example 3

This example demonstrates the in vivo sites of localization of RNA-NPs upon systemic administration and that RNA NPs mediate peripheral and intratumoral activation of DCs.

DOTAP lipid NPs made as essentially described in Example 1 are complexed with Cre recombinase-encoding mRNA to make Cre-encoding RNA-NPs. These multilamellar RNA-NPs are administered to Ai14 transgenic mice, which carry a STOP cassette flanked by IoxP. The STOP cassette prevents the transcription of tdTomato until Cre-recombinase is expressed. A week after RNA-NPs are administered, the lymph nodes, spleens and livers of the transgenic mice are harvested, sectioned and stained with DAPI. The expression of tdTomato is analyzed by fluorescent microscopy following the procedures as essentially described in Sayour et al, Nano Letters 2018. It is expected that the Cre-mRNA-NPs localize in vivo to lymphoid organs, including liver, spleen, and lymph nodes.

DOTAP lipid NPs made as essentially described in Example 1 are complexed with non-specific RNA (e.g., RNA that was not tumor antigen-specific; ovalbumin (OVA) mRNA) and intravenously injected into C57BI/6 mice (n=3-4/group) bearing subcutaneous B16F10 tumors. Lymph nodes, spleens, livers, bone marrow and tumors are harvested within 24 hrs and analyzed for expression of the Dendritic Cell (DC) activation marker, CD86, by CD11c cells (*p<0.05 Mann-Whitney) test). It is expected that the OVA mRNA-NPs demonstrate widespread in vivo localization to the lymph nodes, spleens, livers, bone marrow, and tumors and activated the DCs therein (as shown by the increased expression of the activation marker CD86 on CD11c+ cells). Because activated DCs prime antigen-specific T cell responses, lead to anti-tumor efficacy (with increased TILs) in several tumor models, the anti-tumor efficacy of the multi-lamellar RNA NPs was tested.

### Example 4

This example describes a comparison of the nanoparticles of the present disclosure to cationic RNA lipoplexes and anionic RNA lipoplexes.

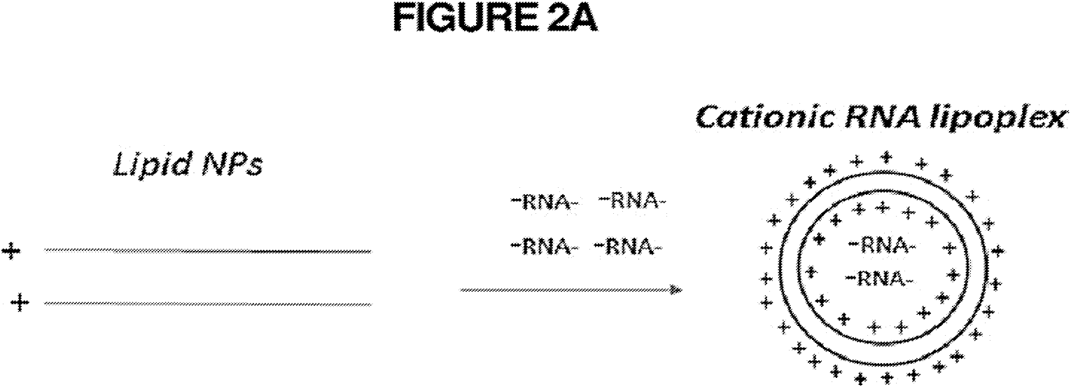

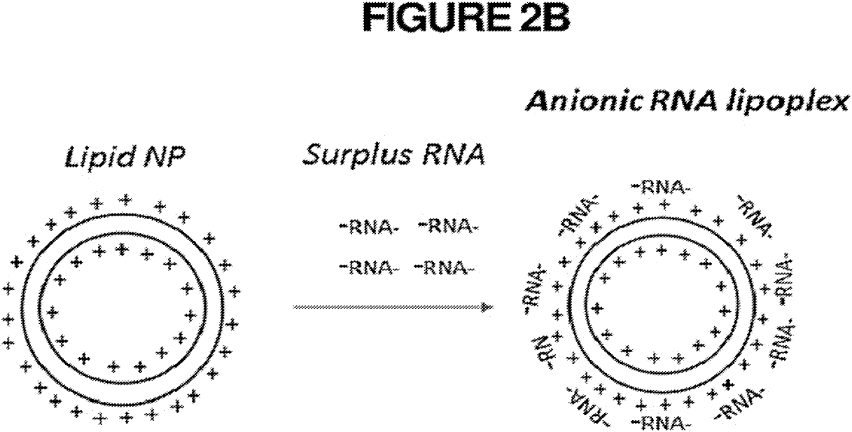

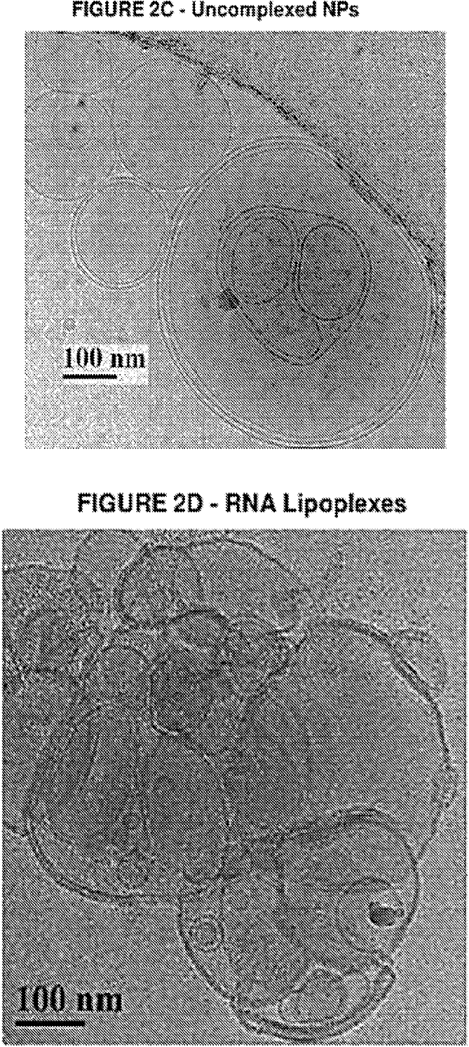

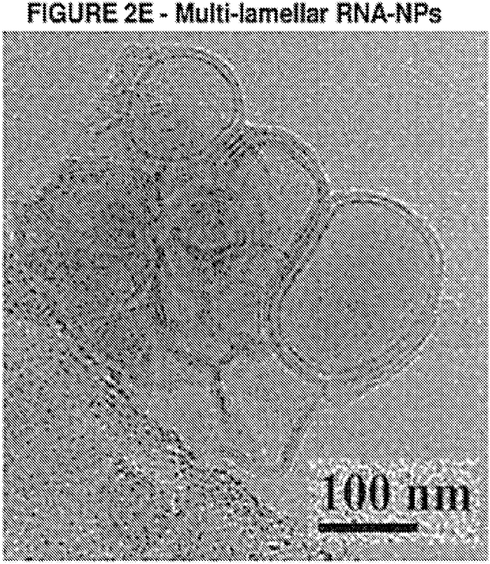

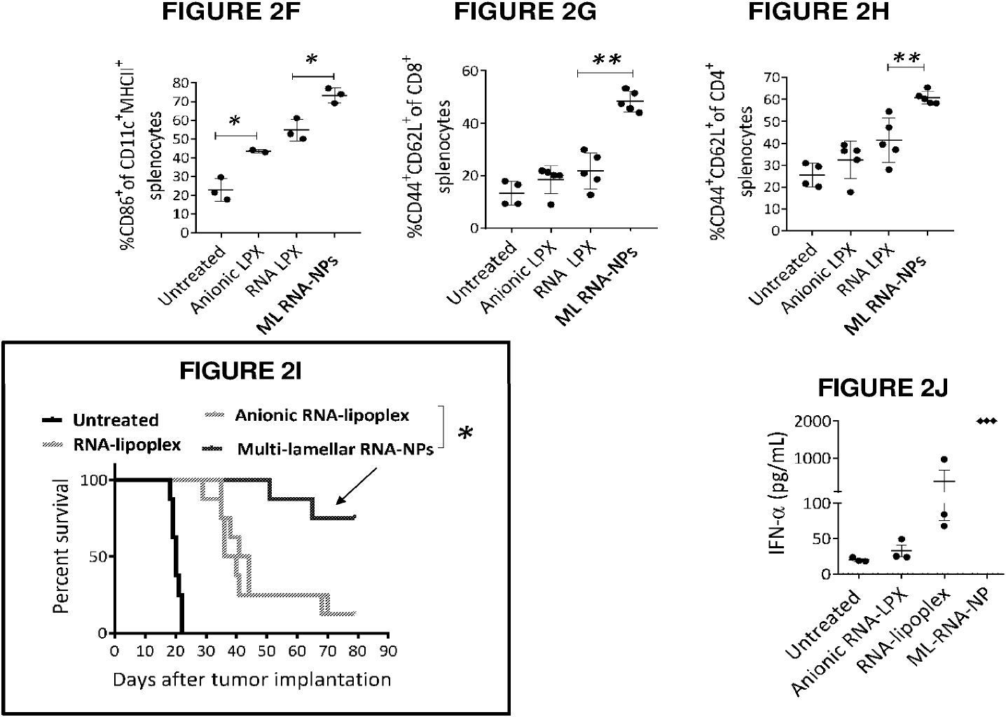

Cationic lipoplexes (LPX) were first developed with mRNA in the lipid core shielded by a net positive charge located on the outer surface (FIG. 2A). Anionic RNA lipoplexes (FIG. 2B) have been developed with an excess of RNA tethered to the surface of bi-lamellar liposomes. RNA-LPX were made by mixing RNA and lipid NP at ratios to equalize charge. Anionic RNA-NPs were made by mixing RNA and lipid NP at ratios to oversaturate lipid NPs with negative charge. Various aspects of the RNA-LPX and anionic RNA LPX were then compared to the multilamellar RNA NPs described in the above examples.

Cryo-Electron Microscopy (CEM) was used to compare the structures of the RNA LPX and the multilamellar RNA-NPs prepared as described in Example 1. Uncomplexed NPs were used as a control. CEM was carried out as essentially described in Example 2. FIG. 2C is a CEM image of uncomplexed NPs, FIG. 2D is a CEM image of RNA LPXs (wherein that mass ratio of liposome to RNA is 3.75:1) and FIG. 2E is a CEM image of the multilamellar RNA-NPs (wherein that mass ratio of liposome to RNA is 15:1). These data support that more RNA is held by the ML RNA-NPs. Additional data show that the concentration drops more with ML RNA-NP complexation versus RNA LPX supporting multilamellar formation of ML RNA-NPs not observed by simple mixing of equivalent amounts of RNA and lipid NPs by mass or charge (i.e. RNA-LPX and anionic RNA-LPX respectively). This supports that more RNA is “held” by ML RNA-NPs.

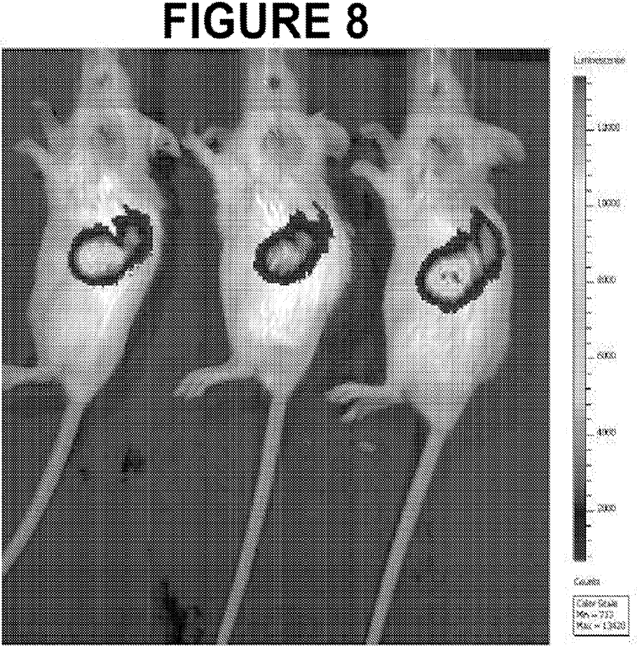

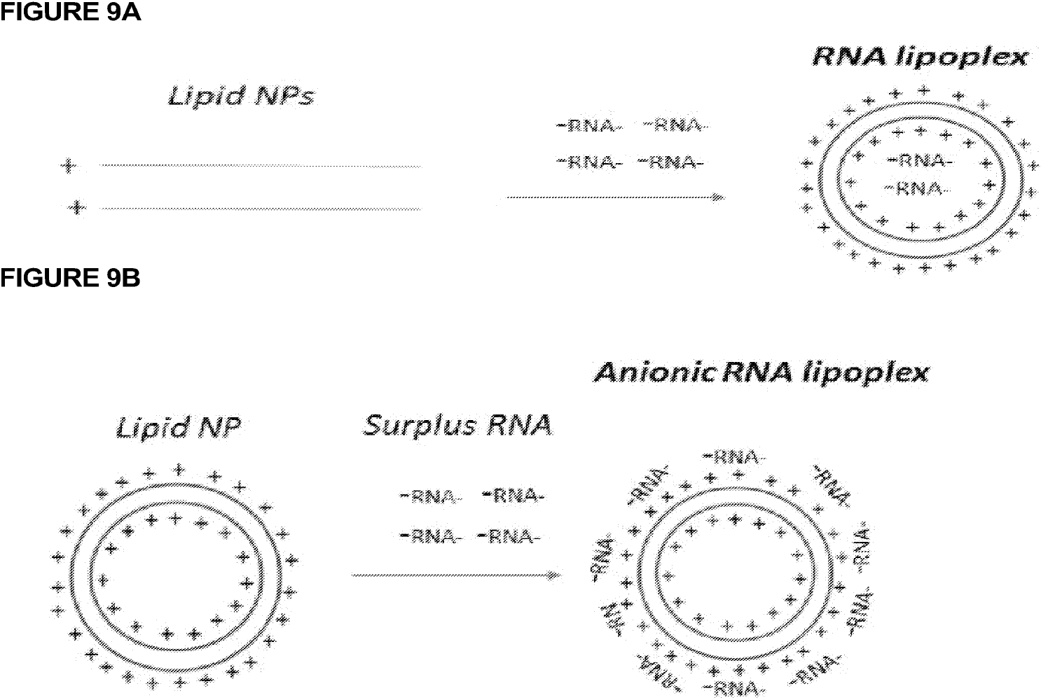

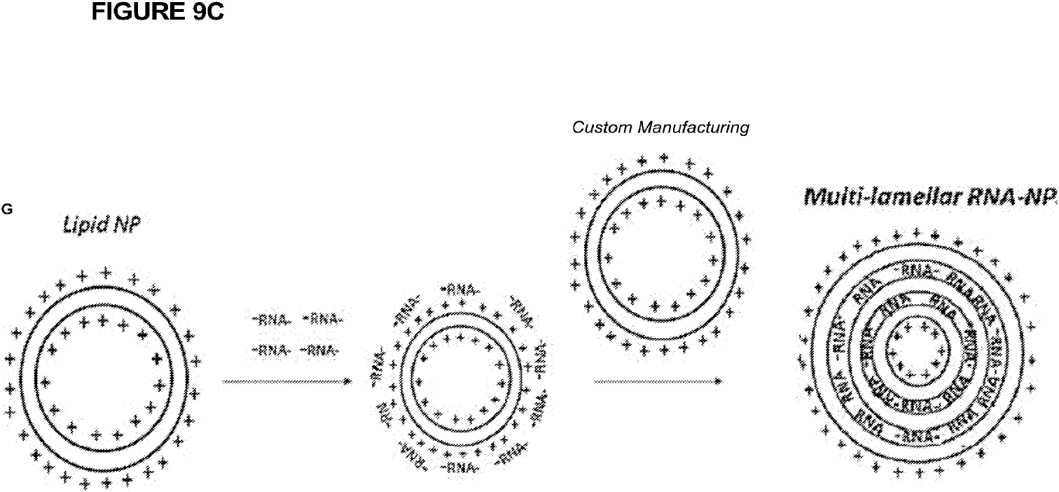

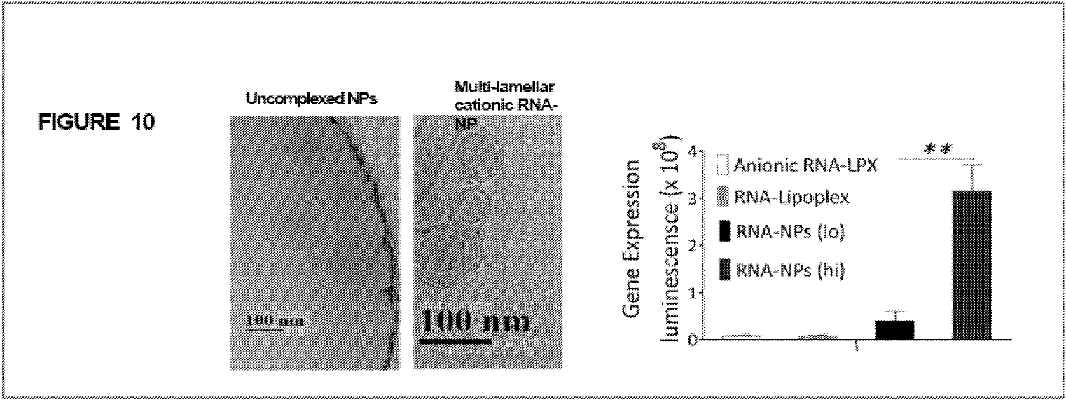

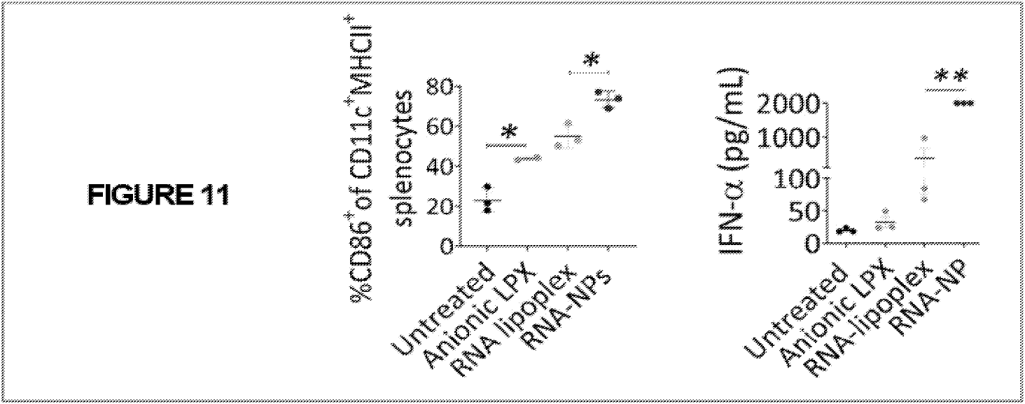

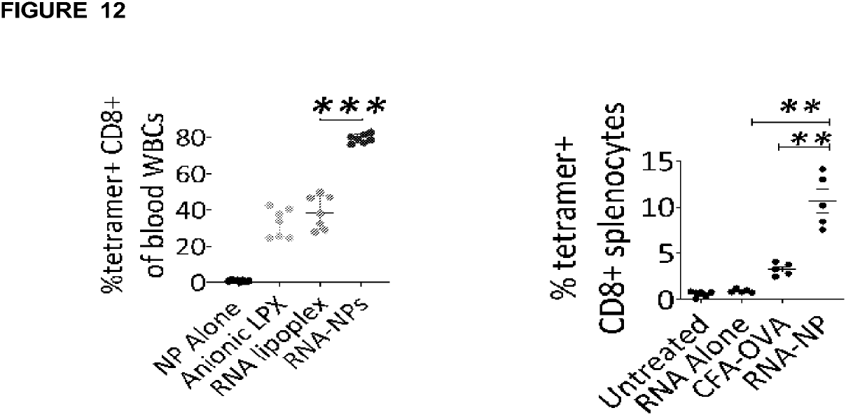

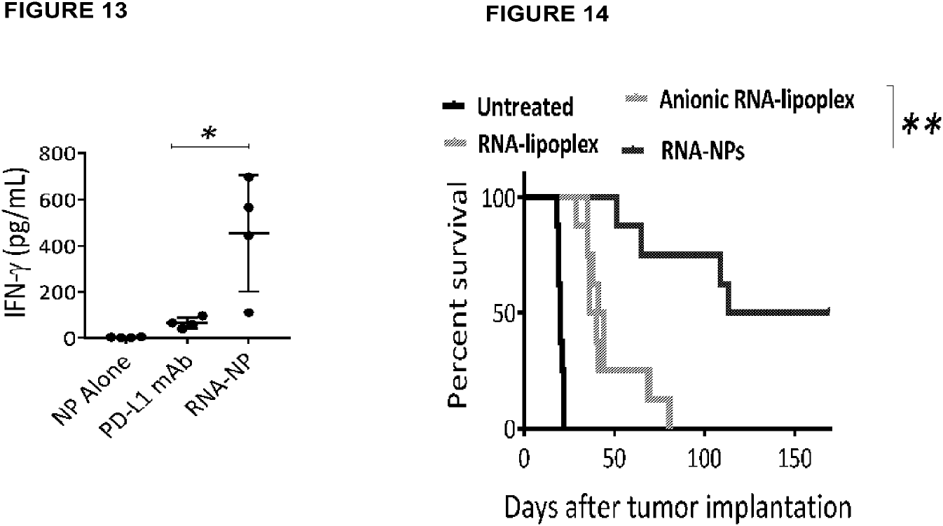

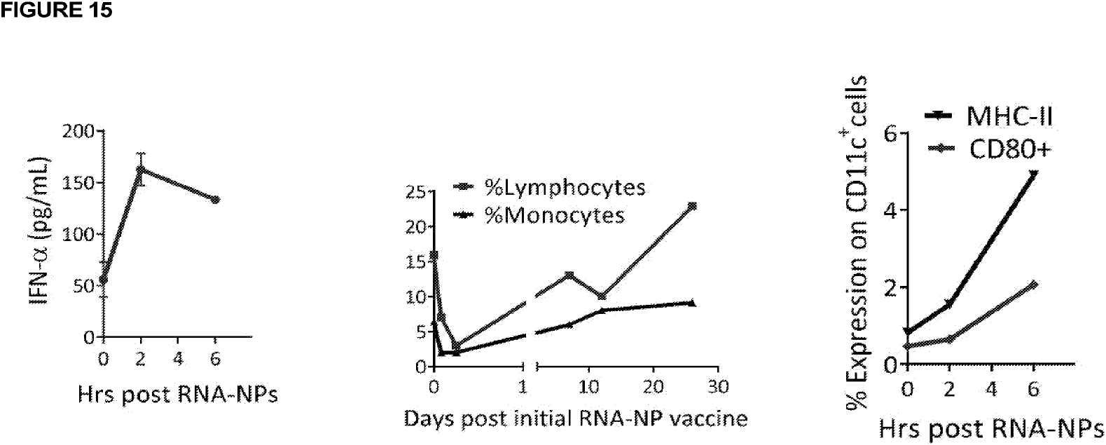

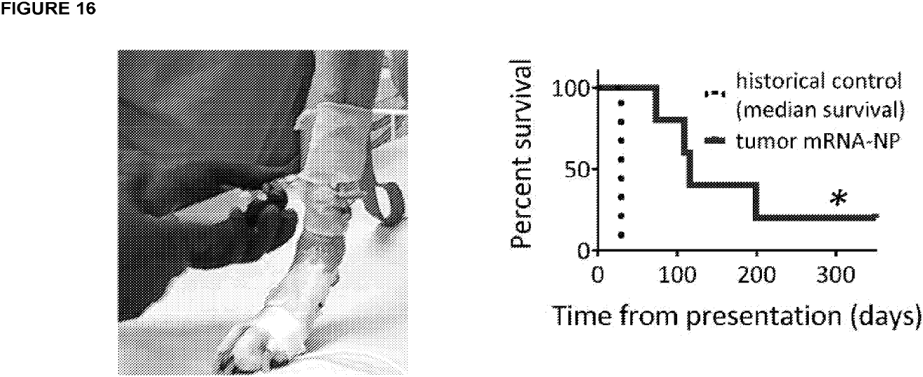

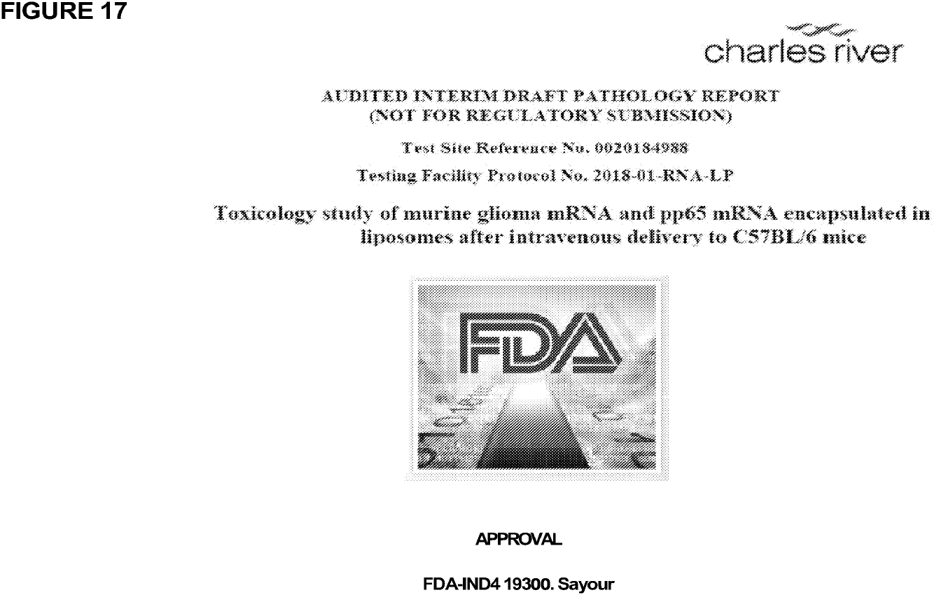

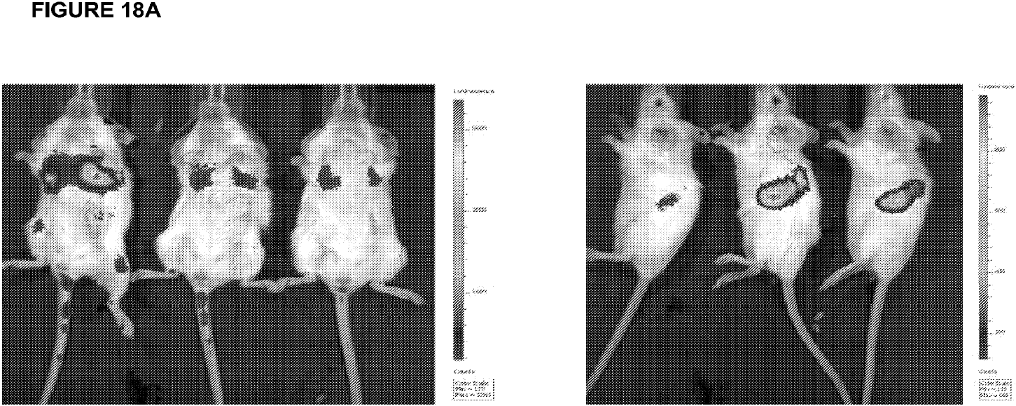

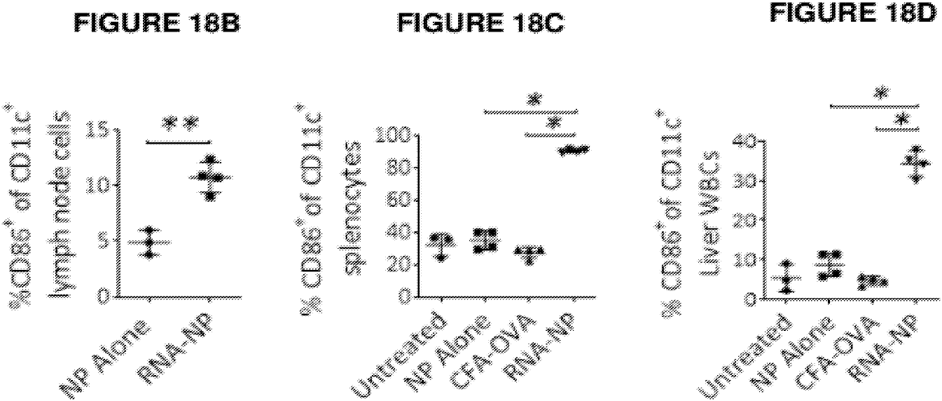

Also, an experiment was conducted to determine where the anionic LPXs localize upon administration to mice. As shown in FIG. 8, anionic LPXs localized to the spleens of animals upon administration, consistent with previous studies (Krantz et al, Nature 534: 396-401 (2016)).

RNA LPX, anionic lipoplex (LPX) or multilamellar RNA-NPs were administered to mice and spleens were harvested one week later for assessment of activated DCs (*p<0.05 unpaired t test). The RNA used in this experiment was tumor-derived mRNA from the K7M2 tumor osteosarcoma cell line. As shown in FIG. 2F, mice treated with multilamellar RNA NPs exhibited the highest levels of activated DCs.

Anionic tumor mRNA-lipoplexes, tumor mRNA-lipoplexes, and multilamellar tumor mRNA loaded NPs were compared in a therapeutic lung cancer model (K7M2) (n=5-8/group). Each vaccine was intravenously administered weekly (x3) (**p<0.01, Mann Whitney). The % CD44+CD62L+of CD8+ splenocytes is shown in FIG. 2G and the % CD44+CD62L+of CD4+ splenocytes is shown in FIG. 2H. Also, FIG. 2J shows that multilamellar (ML) RNA-NPs mediate substantially increased IFN-alpha which is an innate anti-viral cytokine. This demonstrates that ML RNA-NPs allow for substantially greater innate immunity which is enough to drive efficacy from even non-antigen specific ML RNA-NPs. Taken together, these figures demonstrate the superior efficacy of multilamellar tumor specific RNA-NPs, relative to anionic LPX and RNA LPX.

Anionic tumor mRNA-lipoplexes, cationic tumor mRNA-lipoplexes and multilamellar tumor mRNA loaded NPs were compared in a therapeutic lung cancer model (K7M2) (n=8/group). Each vaccine was i.v. administered weekly (x3), *p<0.05, Gehan Breslow-Wilcoxon test. The percent survival was measured by Kaplan-Meier Curve analysis. As shown in FIG. 2I, multilamellar tumor specific RNA-NPs mediated superior efficacy, compared to cationic RNA lipoplexes and anionic RNA lipoplexes, for increasing survival.

Herein it is demonstrated that the multilamellar RNA-NP formulation targeting physiologically relevant tumor antigens is more immunogenic (FIGS. 2F-2H, 2J) and significantly more efficacious (FIG. 2I) compared with anionic LPX and RNA LPX. Without being bound to any particular theory, by altering RNA-lipid ratios and increasing the zeta potential, a novel RNA-NP design composed of multi-lamellar rings of tightly coiled mRNA has been developed (FIG. 1C), which multi-lamellar design is thought to facilitate increased NP uptake of mRNA (condensed by alternating positive/negative charge) for enhanced particle immunogenicity and widespread in vivo localization to the periphery and tumor microenvironment (TME). Systemic administration of these multi-lamellar RNA-NPs localize to lymph nodes, reticuloendothelial organs (i.e. spleen and liver) and to the TME, activating DCs therein (based on increased expression of the activation marker CD86 on CD11c+ cells). These activated DCs prime antigen specific T cell responses, which lead to anti-tumor efficacy (with increased TILs) in several tumor models.

### Example 5

This example demonstrates the ability of multilamellar RNA-NPs to systemically activate DCs, induce antigen specific immunity and elicit anti-tumor efficacy.

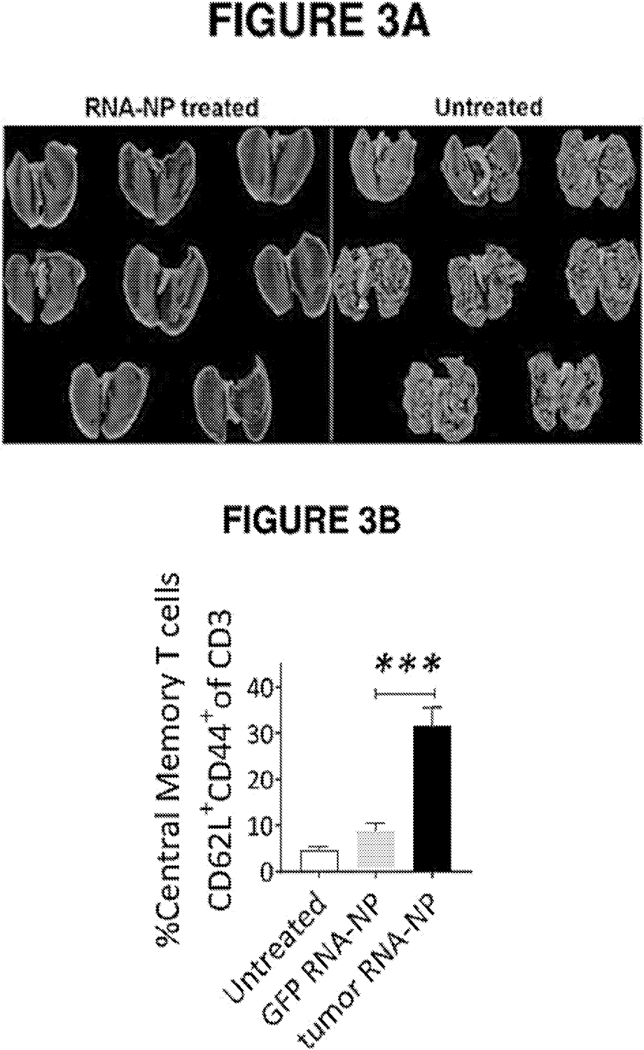

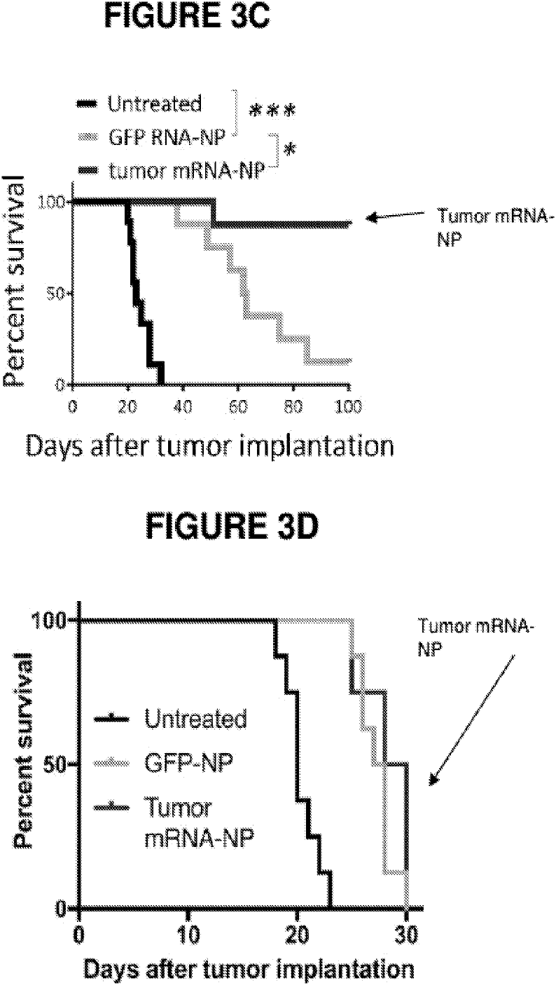

The effect of multilamellar RNA NPs were tested in a second model. Here, BALB/c mice (8 mice per group) inoculated with K7M2 lung tumors were vaccinated thrice-weekly with multilamellar RNA-NPs. A control group of mice was untreated. The lungs were harvested one week after the 3rd vaccine for analysis of intratumoral memory T cells ***p<0.001, Mann Whitney test. FIG. 3A provides a pair of photographs of RNA-NP treated-lungs (left) and of untreated lungs (right). FIG. 3B is a graph of the % central memory T cells (CD62L+CD44+ of CD3+ cells) in the harvested lungs of untreated mice, mice treated multilamellar RNA NPs with GFP RNA, and mice treated multilamellar RNA NPs with tumor-specific RNA.

Also, BALB/c mice or BALB/c SCID (Fox Chase) mice (8 mice per group) were inoculated with K7M2 lung tumors and vaccinated intravenously thrice-weekly with multilamellar RNA-NPs comprising GFP RNA or tumor-specific RNA. A control group of mice was untreated. % survival was plotted on a Kaplan-Meier curve (***p<0.0001, Gehen-Breslow-Wilcox). As shown in FIG. 3C, the percent survival of BALB/c mice treated with multilamellar RNA NPs with tumor-specific RNA was highest among the three groups. Interestingly, the percent survival of BALB/c SCID (Fox Chase) mice treated with multilamellar RNA NPs with GFP RNA was about the same as mice treated with multilamellar RNA NPs with tumor-specific RNA (FIG. 3D).

Taken together, the data of FIGS. 3A-3D demonstrate that monotherapy with RNA-NPs comprising GFP RNA or tumor-specific RNA mediates significant anti-tumor efficacy against metastatic lung tumors in immunocompetent animals and SCID mice. In BALB/c mice bearing metastatic lung tumors (FIGS. 3A-3D), both GFP (control) and tumor specific RNA-NPs mediate innate immunity and anti-tumor activity; however, only tumor specific RNA-NPs mediate increases in intratumoral memory T cells and long-term survivor outcome (FIGS. 3A-3D). Anti-tumor activity of RNA-NPs in mice bearing intracranial malignancies was also demonstrated (data not shown).

These data demonstrate that multilamellar RNA-NPs systemically activate DCs, induce antigen specific immunity and elicit anti-tumor efficacy. FIGS. 3A-3D shows that control RNA-NPs elicit innate response with some efficacy that is not as robust as tumor specific RNA-NPs. Compared with untreated mice, no effects of uncomplexed NPs have been observed, but both non-specific (GFP RNA) and tumor-specific RNA when incorporated into multilamellar RNA NPs mediate innate immunity; however only tumor specific RNA-NPs elicit adaptive immunity that results in a long-term survival benefit (FIGS. 3A-3D). While the data described above relates to anti-tumoral response, the data illustrate the ability of the RNA-NPs of the disclosure induce antigen specific immunity generally.

### Example 6

This example demonstrates personalized tumor RNA-NPs are active in a translational canine model.

The safety and activity of multilamellar RNA-NPs was evaluated in client-owned canines (pet dogs) diagnosed with malignant gliomas or osteosarcomas. The malignant gliomas or osteosarcomas from dogs were first biopsied for generation of personalized tumor RNA-NP vaccines.

To generate personalized multilamellar RNA NPs, total RNA materials was extracted from each patient’s biopsy. A cDNA library was then prepared from the extracted total RNA, and then mRNA was amplified from the cDNA library. mRNA was then complexed with DOTAP lipid NPs, into multilamellar RNA-NPs as essentially described in Example 1. Blood was drawn at baseline, then 2 hours and 6 hours post-vaccination for assessment of PD-L1, MHCII, CD80, and CD86 on CD11c+cells. CD11c expression of PD-L1, MHC-II, PDL1/CD80, and PD-L1/CD86 is plotted over time during the canine’s initial observation period. CD3+ cells were analyzed over time during the canine’s initial observation period for percent CD4 and CD8, and these subsets were assessed for expression of activation markers (i.e. CD44). From these data, it was shown that multilamellar RNA-NPs elicited an increase in 1) CD80 and MHCII on CD11c+ peripheral blood cells demonstrating activation of peripheral DCs; and 2) an increase in activated T cells

Interestingly, within a few hours after administration, tumor specific RNA-NPs elicited margination of peripheral blood mononuclear cells, which increased in the subsequent days and weeks post-treatment, suggesting that RNA-NPs mediate lymphoid honing of immune cell populations before egress.

These data demonstrated that personalized mRNA-NPs are safe and active in translational canine disease models.

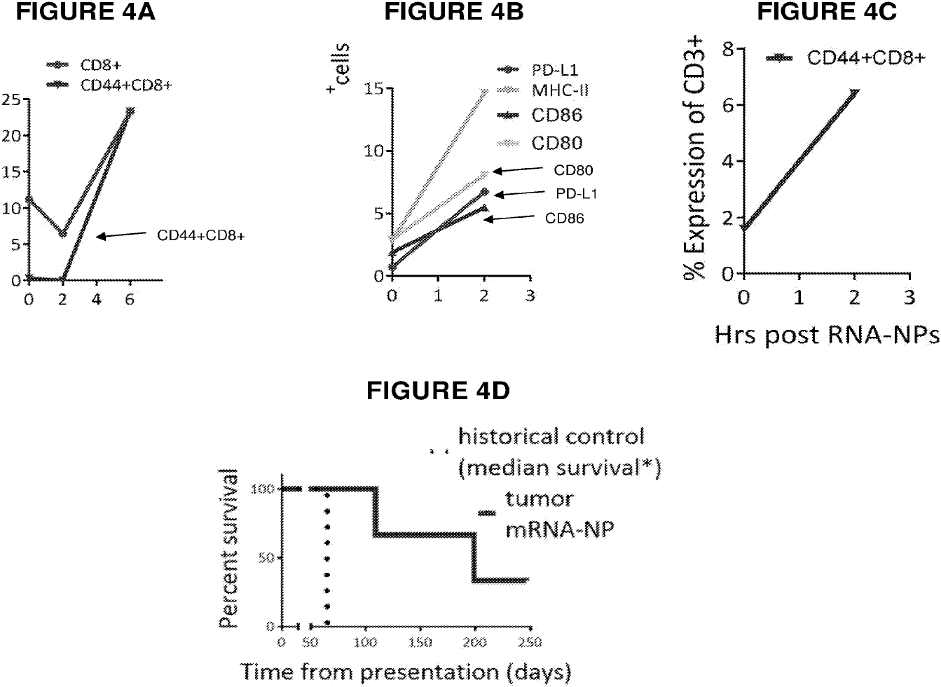

Specific data from canines evaluated in this manner are shown. A 31 kg male Irish Setter was enrolled on study per owner’s consent to receive multilamellar RNA-NPs. Tumor mRNA was successfully extracted and amplified after tumor biopsy. Immunologic response was plotted in response to 1^{st} vaccine. The data show increased activation markers over time on CD11c+ cells (DCs) (FIG. 4A). The data show increased CD8+ cells that are activated (CD44+CD8+ cells) within the first few hours post RNA-NP vaccine. These data support that the multilamellar RNA-NPs are immunologically active in a male Irish Setter. A male boxer diagnosed with a malignant glioma was enrolled on study per owner’s consent to receive RNA-NPs. Tumor mRNA was successfully extracted and amplified after tumor biopsy. Immunologic response is plotted in response to 1^{st} vaccine (FIG. 4B). The data show increased activation markers over time on CD11c+cells (DCs). As shown in FIG. 4C, an increase in activated T cells (CD44+CD8+ cells) was observed within the first few hours post RNA-NP vaccine. These data support that the multilamellar RNA-NPs are immunologically active in a male canine boxer.

After receiving weekly RNA-NPs (x3), the canines diagnosed with malignant gliomas had a steady course. Post vaccination MRI showed stable tumor burdens, with increased swelling and enhancement (in some cases), which may be more consistent with pseudoprogression from an immunotherapeutic response in otherwise asymptomatic canines. Survival of canines diagnosed with malignant gliomas receiving only supportive care and tumor specific RNA-NPs (following tumor biopsy without resection) is shown in FIG. 4D. In FIG. 4D, the median survival (shown as dotted line) was about 65 days and was reported from a meta-analysis of canine brain tumor patients receiving only symptomatic management. In a previous study, cerebral astrocytomas in canines has been reported to have a median overall survival of 77 days. The personalized, multilamellar RNA NPs allowed for survival past 200 days.

Aside from low-grade fevers that spiked 6 hrs post-vaccination on the initial day, personalized tumor RNA-NPs (1x) were well tolerated with stable blood counts, differentials, renal and liver function tests. To date, we have treated four client-owned canines diagnosed with malignant brain tumors. It is important to highlight that these canines received no other therapeutic interventions for their malignancies (i.e., surgery, radiation or chemotherapy), and all patients assessed developed immunologic response with pseudoprogression or stable/smaller tumors. We have not appreciated significant toxicities in canines that would preclude investigation in humans at 1x dosing based on clinical presentation, physical exam findings, and laboratory tests. One canine was autopsied after RNA-NP vaccines. In this patient, there were no toxicities believed to be related to the interventional agent.

These results suggest safety and activity of tumor specific RNA-NPs in client-owned canines with malignant brain tumors for subjects that did not receive any other anti-tumor therapeutic interventions.

### Example 7

This example demonstrates toxicology study of murine glioma mRNA and pp65 mRNA encapsulated in DOTAP liposomes after intravenous delivery to C57BL/6 mice.

The objective of this study was to evaluate the safety of pp65 mRNA encapsulated by DOTAP liposomes when delivered intravenously in C57BL/6 mice. Experimental procedures applicable to pathology investigations are summarized in Table 1. All interim phase animals were submitted for necropsy on Day 35±1 day. Tissue samples listed in Table 2 were collected and fixed in 10% neutral buffered formalin, except for optic tissue and testis tissue, which was fixed in Davidson’s solution; tissues from the early death animal were fixed in 10% neutral buffered formalin.

TABLE 1

| | | | Number of Mice | | | | | |
|---|---|---|---|---|---|---|---|---|
| | | | Day 35±1 day | | Day 56±2 days | | Day 112±3 days | |
| | | Total Dose | Males | Females | Males | Females | Males | Females |
| 1 | Vehicle | 0 | 5 | 5 | 5 | 5 | 5 | 5 |
| 2 | LP | 0 + 15.0 | 5 | 5 | 5 | 5 | 5 | 5 |
| 3 | RNA + LP | 0.2 + 3.0 | 5 | 5 | 5 | 5 | 5 | 5 |
| 4 | RNA + LP | 1.0 + 15.0 | 5 | 5 | 5 | 5 | 5 | 5 |

TABLE 2

Tissue Collection and Examination

| | | | |
|---|---|---|---|
| Provantis Tissue Term | Protocol Tissue Term | Collect | Microscopic Evaluation |
| BONE, FEMUR | Femur with bone marrow (R) | X | X |
| BONE MARROW | | X | X |
| BONE, STERNUM | Sternum | X | X |
| BRAIN | Brain stem | X | X |
| | Cerebellum | | |
| | Cerebrum | | |
| EPIDIDYMIS | Epididymis | X | X |
| ESOPHAGUS | Esophagus | X | X |
| EYE | Eye with optic nerve (R) | X | X |
| NERVE, OPTIC | | X | X |
| GLAND, ADRENAL | Adrenal gland (R) | X | X |
| GLAND, PARATHYROID | Thyroid/parathyroid gland | X | X |
| GLAND, THYROID | | X | X |
| GLAND, PITUITARY | Pituitary | X | X |
| GLAND, PROSTATE | Prostate | X | X |
| GLAND, SALIVARY | Salivary gland (R, mandibular) | X | X |
| GLAND, SEMINAL VESICLE | Seminal vesicles | X | X |
| HEART | Heart | X | X |
| KIDNEY | Kidney (R) | X | X |
| LARGE INTESTINE, CECUM | Cecum | X | X |
| LARGE INTESTINE, COLON | Colon | X | X |
| LARGE INTESTINE, RECTUM | Rectum | X | X |
| LIVER | Liver | X | X |
| LUNG | Lungs | X | X |
| LYMPH NODE, MESENTERIC | Lymph node (mesenteric) | X | X |
| MUSCLE, DIAPHRAGM | Diaphragm | X | X |
| MUSCLE, QUADRICEPS | Quadriceps (R) | X | X |
| NERVE, SCIATIC | Sciatic nerve (R) | X | X |
| OVARY | Gonad (Ovary, R) | X | X |
| PANCREAS | Pancreas | X | X |
| SITE, INJECTION | Tail (injection site) | X | X |
| SKIN | Skin | X | X |
| SMALL INTESTINE, DUODENUM | Duodenum | X | X |
| SMALL INTESTINE, ILEUM | Ileum | X | X |
| SMALL INTESTINE, JEJUNUM | Jejunum | X | X |
| SPINAL CORD | Spinal cord, cervical Spinal cord, lumbar Spinal cord, thoracic | X | X |
| SPLEEN | Spleen | X | X |
| STOMACH | Stomach | X | X |
| TESTIS | Gonad (Testis, R) | X | X |
| THYMUS | Thymus | X | X |
| TONGUE | Tongue | X | X |
| URINARY BLADDER | Urinary bladder | X | X |
| UTERUS | Uterus | X | X |
| VAGINA | Vagina | X | X |
| - | Gross lesions | X | X |

Tissues required for microscopic evaluation were trimmed, processed routinely, embedded in paraffin, and stained with hematoxylin and eosin by Charles River Laboratories Inc., Skokie, Illinois. Light microscopic evaluation was conducted by the Contributing Scientist, a board-certified veterinary pathologist on all protocol-specified tissues from all animals in Groups 1 and 4, and any early death animals.

Tissues that were supposed to be microscopically evaluated per protocol but were not available on the slide (and therefore not evaluated) are listed in the Individual Animal Data of the pathology report as not present. These missing tissues did not affect the outcome or interpretation of the pathology portion of the study because the number of tissues examined from each treatment group was sufficient for interpretation.

Gross Pathology: No test article-related gross findings were noted. The gross findings observed were considered incidental, of the nature commonly observed in this strain and age of mouse, and/or were of similar incidence in control and treated animals and, therefore, were considered unrelated to administration of a 1:1 ratio of pp65 mRNA and KR158mRNA in DOTAP liposomes.

Histopathology: No test article-related microscopic findings were noted. There were a few animals with inflammatory cell infiltrates at the injection site; this finding is common for injection sites and at this point in the study, was considered equivocal. The microscopic findings observed were considered incidental, of the nature commonly observed in this strain and age of mouse, and/or were of similar incidence and severity in control and treated animals and, therefore, were considered unrelated to administration of a 1:1 ratio of pp65 mRNA and KR158mRNA in DOTAP liposomes.

It was concluded that intravenous injection into the tail vein of mice of 1.0 mg/kg KR158 and pp65 mRNAs + 15.0 mg/kg DOTAP liposome on Study Days 0, 14, and 28 resulted in no gross or microscopic test article-related findings on Study Day 35±1 day. There were small amounts of inflammatory cell infiltrates at the injection site, which is a common finding for injection sites. This finding was equivocal.

### Example 8

This example describes a study aimed at determining the impact of pDCs transfected with multilamellar RNA-NPs on antigen specific T-cell priming.

While pDCs are well-known stimulators of innate immunity and type IIFN, they also mediate profound effects on intratumoral adaptive immunity. They can: 1) directly present antigen for priming of tumor specific T cells; 2) assist adaptive response through chemokine recruitment of other DC subtypes (via chemokines CCL3, CCL4, CXCL10); 3) polarize Th1 immunity through IL-12 secretion; and/or 4) mediate tumor antigen shedding (through cytokine, TRAIL or granzyme B) for DC loading and T cell priming. Despite these effector functions, pDCs may also dampen immunity through release of immunoregulatory molecules (IL-10, TGF-β, and IDO) and promotion of regulatory T cells (Tregs). The purpose of this study is to elucidate the effects of RNA-NP transfected-pDCs on adaptive immunity and antigen specific T cell priming. It is hypothesized that RNA-NP activated pDCs serve as direct primers of antigen specific immunity and assist classical DCs (cDCs) and/or myeloid-derived DCs (mDCs) in promoting effector T-cell response. These experiments are to shed new light on the activation state of pDCs requisite for RNA-NP mediated immunity and their exhaustion over time that may be co-opted for enhanced immunotherapeutic effect.

### Statistical Analyses

In the study of Example 9.1 where survival is of interest, the log-rank test is used to compare Kaplan-Meier survival curves between treatment groups and control groups. Experience with our tumor models indicates that median overall survival in untreated control mice is approximately 30 days, with survival times following a Weibull distribution with shape parameter k=6. As an example, with 10 mice each in 2 tumor-bearing groups (treated and untreated), comparison of survival curves using a one-sided log-rank test evaluated at 0.05 significance has at least 80% power to detect an improvement in median survival of 8 days in the treated group compared to the untreated group. This effect size was determined by simulating 1000 Weibull-distributed survival datasets with shape parameter k=6 under the alternative hypothesis effect size and then observed the proportion of log-rank tests of these datasets that were significant at p<0.05. In the studies of Examples 9.2-9.4, responses observed at different times are analyzed using a two-way ANOVA model with mutually exclusive groups distributed among treatments and observation times. change in immune response parameters over time are assessed using generalized linear mixed effect models (GLMMs). Response variables for experiments that are completely replicated at least once are analyzed using GLMMs. Experimental replication is modeled as a random effect to account for “batch” or “laboratory day” variability. Treatment and control groups are modeled as fixed effects and compared using ANOVA-type designs nested within the mixed effect modeling framework.

### Example 8.1

This example describes an experiment designed to determine anti-tumor efficacy of RNA-NPs in wild-type and pDC KO mice.

Tumorgenicities for KR158b-luc, GL261-luc and a murine H3.3K27M mutant cell line have been set up. KR158b-luc and GL261-Iuc are both transfected with luciferase so that tumors can be monitored for growth using bioluminescent imaging. Tumorigenic dose of KR158b-luc and the H3K27M mutant line is 1×10⁴ cells. Tumorigenic dose of GL261-luc is 1×10⁵ cells. GL261 and KR158 are injected into the cerebral cortex of C57BI/6 (3 mm deep into the brain at a site 2 mm to the right of the bregma); H3K27M glioma cells are injected midline. Tumor mRNA is extracted from the parental cell lines (i.e. KR158b without luciferase) for vaccine formulation consisting of an intravenous (iv) injection of 25 µg of tumor specific mRNA complexed with 375 µg of our custom lipid-NP formulation (per mouse). These are compared simultaneously to 10 negative control mice receiving NPs alone and nonspecific (i.e. pp65 mRNA) RNA-NPs. Mice are vaccinated 3 times at 7-day intervals beginning 5 days after tumor implantation. IFN-α levels are assessed from serum of wild-type and pDC KO mice at serial time points (5 d, 12 d, and 19 d). In wild-type mice who develop treatment response, but succumb to disease, the immunologic escape mechanisms in tumors (i.e., expression of checkpoint ligands, IDO, downregulation of MHC class I) and within the tumor microenvironment (i.e., MDSCs, Tregs, and TAMs) are explored.

Based on preclinical data demonstrating anti-tumor activity of RNA-NPs in these models, it is anticipated that anti-tumor activity is abrogated in pDC KO mice.

### Example 8.2

This example describes an experiment designed to determine the pDC phenotype and function following activation by RNA-NPs.

To assess pDC phenotype, KR158b bearing C57BI/6 mice are vaccinated with TTRNA-NPs composed from 375 µg of FITC labeled DOTAP (Avanti) with 25 µg of TTRNA (derived from KR158b and delivered iv). Twenty-four hours after vaccination recipient mice are euthanized (humanely killed with CO2) for collection of spleens, tumor draining lymph nodes (tdLNs) and tumors. Organs are digested into a single cell suspension, undergo RBC lysis (PharmLyse, BD Bioscience) before incubation at 37° C. for 5 minutes. Ficoll gradients are used to separate WBCs from parenchymal cells. The cells at the interface are collected, washed, and analyzed. pDCs are stained for CD11c, B220 and Gr-1 (ebioscience). Distinct pDC subsets are identified by differential staining for CCR9, SCA1, and Ly49q. Activation state is assessed based on expression of co-stimulatory molecules (i.e. CD40, CD80, CD86) chemokines (i.e. CCL3, CCL4, CXCL10) and chemokine receptors (i.e. CCR2, CCR5, CCR7). Detection secondary antibody is rabbit IgG conjugated with AlexaFlour®488 (ThermoFisher Scientific) for FITC detection. Effector versus regulatory function is determined through intracellular staining for effector (i.e. IFN-I, IL-12) versus regulatory cytokines (i.e. TGF-β, IL-10). Analyses will be conducted by multi-parameter flow cytometry (LSR, BD Bioscience) and immunohistochemistry (IHC).

Based on our preliminary data showing substantial increases in pDCs in peripheral and intratumoral organs, it is expected to identify FITC positive pDCs in the spleen, tdLNs and intracranial tumors.

### Example 8.3

This example describes an experiment designed to determine whether RNA-NP transfected pDCs mediate direct or indirect activation of antigen specific T cells.

While pDCs are well known stimulators of innate immunity and type IIFN, their cumulative effects on antigen specific responses are still being uncovered. Since they express MHC class II, they have APC capacity, but compared to their cDC counterparts, they are believed to be poor direct primers of antigen specific immunity. This experiment is aimed at yielding a better understanding of pDCs, in the context of RNA-NPs, as either direct primers or facilitators of antigens specific immunity. To determine the effects of pDCs on antigen specific T cells, KR158b bearing mice are vaccinated with TTRNA (derived from the murine glioma line KR158b) encapsulated into FITC-labeled NPs (Avanti), and FACSort (BD Aria II) relevant FITC+ pDCs from spleens, tdLNs and intracranial tumors (as indicated above). RNA-NP transfected pDCs are then co-cultured with naïve magnetically separated CD4 and CD8 T cells, and T cells are assessed for proliferation, phenotype (effector vs central memory), function and cytotoxicity. Indirect effects from pDCs are assessed via ex vivo co-cultures with TTRNA-loaded DCs (matured ex vivo from murine bone marrow) with naïve CD4 and CD8 T cells. Ex vivo co-cultures will be performed in triplicate, for 7 days in a 96 well plate with naïve T cells (40,000 RNA-NP transfected pDCs with 400,000 T cells) labeled with CFSE (Celltrace, Life Technologies). T cell proliferation is determined by measuring CFSE dilution by flow cytometry. Phenotype for effector and central memory populations is determined through differential staining for CD44 and CD62L. These T cells are re-stimulated for a total of two cycles before supernatants are harvested for detection of Th1 cytokines (i.e. IL-2, TNF-α, and IFN-y) by bead array (BD Biosciences). Stimulated T cells are also incubated in the presence of KR158b (stably transfected with GFP) or control tumor (B16F10-GFP) and assessed for their ability to induce cytotoxicity. Amount of GFP in each co-culture, as a surrogate for living tumor cells, are quantitatively measured by flow cytometry.

The in vivo effects of FACSorted RNA-NP transfected pDCs are determined by adoptively transferring these cells (250,000 cells/mouse) to tumor-bearing mice (weekly x3) and harvesting spleens, tdLNs, and tumors one week later for assessment of antigen specific T cells by YFP expression in IFN-y reporter mice (GREAT mice, B6 transgenic, containing IFN-y promotor with IRES-eYFP reporter, Jackson labs). In separate experiments, IFN-y reporter mice are vaccinated with TTRNA-NPs with and without pDC depleting mAbs before harvesting spleens, tdLNs, and intracranial tumors one week later for determination of antigen specific T cells by YFP expression. T cell functional assays are performed as described above.

It is anticipated that these pDCs are requisite for priming antigen specific T cells through either direct and/or indirect means.

### Example 8.4

This example describes an experiment designed to determine whether RNA-NP activated pDCs promote antigen specific T cell priming from cDCs and/or mDCs.

While IFN-I release from pDCs is known to increase activation markers on cDCs and mDCs, the role of pDCs on direct T cell priming from cDCs/mDCs is less clear. This experiment is aimed at elucidating the ability of RNA transfected cDCs and mDCs to prime antigen specific T cells in the presence or absence of activated pDCs. To determine effects of pDCs on other DC subsets, KR158b bearing C57BI/6 and pDC knock out (KO) mice (BDCA2-DTR, B6 transgenic mice, Jackson labs) are vaccinated and T cell priming from cDCs and mDCs are assessed. FITC+cDC and mDC populations are sorted via FACSort within 24 h of i.v. TTRNA-NPs (FITC-labeled) and are evaluated for their ability to prime naïve T cell responses in vitro based on proliferation, functional and cytotoxicity assays. Resident and migratory cDCs are identified by CD11c+CD103+MHCII+cells and CD11c+CD11b+MHCII+cells respectively; mDCs are identified by CD11c+CD14+ MHCII+ cells. Cytokines, chemokines and activation markers are analyzed as described in Example 9.1. In vivo effects of these cDC/mDC are carried out in cell transfer experiments as described in Example 9.2. Briefly, FACSorted cDCs and mDCs from TTRNA-NP vaccinated C57BI/6 mice or pDC KO mice are adoptively transferred (250,000 cells/mouse) to tumor-bearing mice (once weekly x3) before harvesting spleens, tdLNs, and intracranial tumors one week later for assessment of antigen specific T cells by YFP expression in IFN-y reporter mice. Proliferation, functional and cytotoxicity assays are performed.

It is expected that ML RNA-NPs activate pDCs which enhance activation phenotype and direct priming of T cells from cDCs and mDCs.

If a lack of indirect effects from pDCs on cDCs and/or mDCs, pDC’s effects on NK cells are evaluated including their activation state, function, and cytotoxicity.

### Example 8.5

This example describes an experiment designed to determine how pDCs influence effector/regulatory T cells over time within the intratumoral microenvironment.

Recruitment of pDCs to tumors is typically associated with a regulatory phenotype characterized by increased IDO, FoxP3+Tregs and secretion of immunoregulatory cytokines. In this experiment, it is determined whether RNA-NP activated pDCs function distinctly by activating T cells over time in the tumor microenvironment. To determine intratumoral effects of pDCs, TTRNA-NPs are administered to KR158b bearing IFN-y reporter mice with and without pDC depleting mAbs (Bioxcell). Activated and regulatory T cells are assessed over time in the intratumoral microenvironment at serial time points (6h, 1d, 7d, and 21d). Effector T cells are characterized, and Tregs are phenotyped through expression of FoxP3, CD25, and CD4. pDCs from non-depleted animals will be FACSorted from these sites and are phenotyped for expression of cytokines, chemokines, activation markers (i.e., CD80, CD86, CD40), cytolytic markers (i.e., TRAIL, granzyme b) and regulatory markers (i.e., IL-10, TGF-β, IDO). Immunophenotypic changes by tumor cells are also assessed over time (i.e., MHC-I, PD-L1, SIRPα).

### Example 9

This example describes a study aimed at evaluating the role of type I interferons on RNA-NP activated T-cell egress, trafficking and function.

Statistical Analysis Tumor-bearing mice are randomized prior to receiving interventional treatments. The choice of 10 animals per group should yield adequate power for detecting effects of interest. As an example, within an ANOVA design with 7 treatment groups observed at a particular time, a pairwise contrast performed within the ANOVA framework can detect an effect size equal to 1.27 SD units with 80% power at a 2-sided significance level of 0.05. Immune parameter responses observed in experimental groups at several observation times are analyzed using generalized linear models (GLMs) with normal or negative binomial response errors. Responses are organized in a two-way ANOVA design with mutually exclusive groups distributed among treatments and observation times. Response variables for experiments that are completely replicated at least once are analyzed using GLMMs. Experimental replication is modeled as a random effect to account for “batch” or “laboratory day” variability. Treatment and control groups are modeled as fixed effects and compared using ANOVA-type designs nested within the mixed effect modeling framework.

### Example 9.1

This example describes an experiment designed to determine the chemokine receptor, S1P1, and VLA-4/LFA-1 expression profile of antigen specific T cells after RNA-NP vaccination.

IFN-I′s effects on sphingosine-1-phosphate receptor 1 (S1P1), which is necessary for T cell egress from lymphoid organs, and integrins (i.e., VLA-4, LFA-1) necessary for T cell traversion across the BBB are assessed. KR158b bearing IFN-γ reporter mice, or IFN-γ reporter mice receiving IFNAR1 blocking mAbs (Bioxcell) are implanted with TTRNA-NPs. RNA-NPs composed from 375 µg of DOTAP (Avanti) with 25 µg of TTRNA (extracted from KR158b and delivered iv) are administered once weekly (x3) and are begun 5 days after implantation. One week after the last vaccine, recipient mice are euthanized (humanely killed with CO₂) and spleens, tdLNs, bone marrow, and intracranial tumors are harvested. Organs are digested, and antigen specific T cells from spleens, lymph nodes, bone marrow and tumors are identified by YFP expression and by differential staining for effector and central memory T cells (i.e., of CD62L and CD44) at serial time points (7, 14 and 21 days). Th1-associated chemokine receptors (i.e., CCR2, CCR5, CCR7 and CXCR3), S1P1 expression, VLA-4, and LFA-1 expression (ebioscience) from CD4 and CD8 T cells are assessed by multi-para meter flow cytometry and IHC.

It is expected that LFA-1 and CCR2 are expressed on activated T cells following RNA-NP administration. If no changes in chemokine expression pattern, S1P1 and integrins on activated T cells after IFNAR1 mAbs, RNA-seq analysis is performed on FACS sorted T cells (YFP+cells) from mice treated with and without IFNAR1 mAbs and assess changes in immune related genes.

### Example 9.2

This example describes an experiment designed to determine the effects of IFN-I on in vitro and in vivo migration of RNA-NP activated T cells.

Based on our data demonstrating increased antigen specific T cells in peripheral organs but lack of anti-tumor efficacy after IFNAR1 blockade, IFN-γ′s effects on RNA-NP activated T cell migration are determined. KR158b bearing IFN-γ reporter mice, or IFN-γ reporter mice receiving IFNAR1, LFA-1 or CCR2 blocking antibodies are vaccinated with iv TTRNA-NPs once weekly (x3). In vivo traversion across the BBB is assessed from percentage and absolute numbers of T cells in intracranial tumors (relative to spleen, lymph nodes and bone marrow) at serial time points (5 d, 10 d, 15 d, 20 d post RNA-NPs).

The migratory capacity of T cells is also analyzed via in vitro cultures. KR158b tumor bearing naïve, INFAR1, LFA-1 or CCR2 KO animals (B6 transgenic, Jackson) are vaccinated with iv TTRNA-NPs. T cells are FACSorted via a BD Aria II Cell Sorter into a 50-100% FBS solution. These T cells are assessed for migratory capacity in transwell assays (ThermoFisher Scientific). Briefly, T cells are placed in the upper layer of a cell culture insert with a permeable membrane in between a layer of KR158b-GFP tumor cells. Migration is assessed by number of cells that shift between layers. T cells are plated in T cell media with and without IL-2 (1 microgram/mL) at a concentration of 4×10⁶ per mL for co-culture with tumor cells (4×10⁶/mL) (×48 hrs) before determination of IFN-γ by ELISA (ebioscience). Amount of GFP in each co-culture, as a surrogate for living tumor cells, is quantitatively measured by flow cytometric analysis.

It is anticipated that type IIFNs are necessary for activated T cell trafficking across the BBB. If there is an inability to adequately define antigen specific T cells, the response against a physiologically relevant GBM antigen, pp65, which will be spiked into our tumor mRNA cohort, is tracked in HLA-A2 transgenic mice by overlapping peptide pool re-stimulation assays and through analysis for pp65-HLA-A2 restricted epitope NTUDGDDNNDV by tetramer staining for CD8+ cells in spleens, tdLNs and intracranial tumors.

### Example 9.3

This example describes an experiment designed to delineate the contribution of IFN-I on antigen specific T cell function following RNA-NPs.

IFN-Is have been shown to promote Tregs and regulate effector and memory CD8+ cells (56), but they are also essential in promoting activated T cell responses following RNA-NP vaccination. Due to these distinct effects, the contribution of IFN-I on antigen specific T cell function following RNA-NP vaccines is determined. KR158b bearing IFN-y reporter mice, or IFN-y reporter mice receiving IFNAR1 mAbs, are vaccinated with iv TTRNA-NPs once weekly (x3). Antigen specific T cells are assessed by YFP+ cells. YFP+T cells from spleens, lymph nodes, bone marrow and tumor are assessed for their activation status (i.e. CD107a, perforin, granzyme), proliferation (through fluorescent dilution of adoptively transferred cells labeled with CellTrace Violet), differentiation (into effector and central memory subsets, and cytotoxicity. T cell cytotoxicity is determined in the presence of KR158b (stably transfected with GFP) or control tumor (B16F10). It is also expected that type IIFNs enhance T cell proliferation and function within the tumor microenvironment.

If no changes in migratory capacity or function of antigen specific T cells after blockade of type IIFN, the effects of type IIFN on modulating T cell exhaustion is assessed. the effects of type IIFNs on expression of immune checkpoints (i.e. PD-1, TIM-3, LAG-3) and their ligands on tumor cells and APCs (i.e. PD-L1, galectin-9) is also evaluated.

### Example 10

This example demonstrates non-antigen specific multilamellar (ML) RNA NPs mediate antigen-specific immunity long enough to confer memory and fend off re-challenge of tumor.

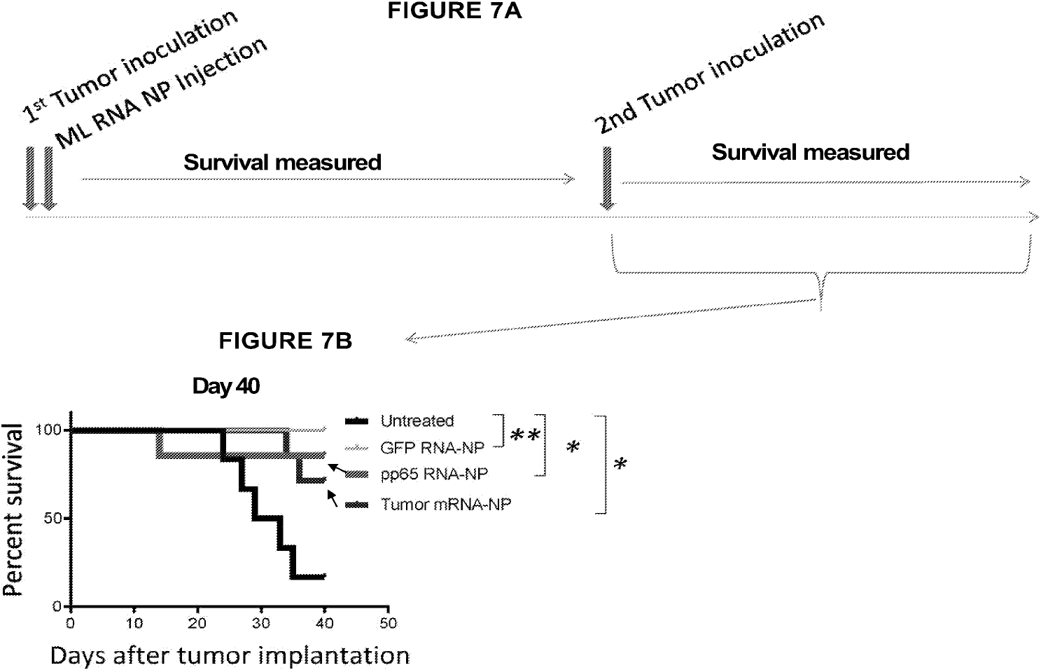

An experiment was carried out with long-term surviving mice (e.g., mice that survived for ~100 days) that were challenged a total of two times via tumor inoculation, but treated only once weekly (x3) with ML RNA NPs comprising GFP RNA or pp65 RNA (each of which were non-specific to the tumor) or with ML RNA NPs comprising tumor-specific RNA. The treatment occurred just after the first tumor inoculation and about 100 days before the second tumor inoculation. Because none of the control mice (untreated mice) survived to 100 days, a new control group of mice were created by inoculating the same type of mice with K7M2 tumors. The new control group like the original control mice did not receive any treatment. The long-time survivors also did not receive any treatment after the second time of tumor inoculation. A timeline of the events of this experiment are depicted in FIG. 7A.

Remarkably, mice in all three groups contained long-time survivors that survived the second tumor challenge. As shown in FIG. 7B (which shows only the time period following the 2^{nd} inoculation), mice in all three groups contained long-time survivors with survival to 40 days post tumor implantation (second instance of tumor inoculation). Interestingly, the percentage of long-time survivor mice that were previously treated with ML RNA NPs comprising non-specific RNA (GFP RNA or pp65 RNA) survived to 40 days post second tumor inoculation, comparable to the group treated with ML RNA NPs comprising tumor specific RNA (treated before second tumor challenge).

These data support that ML RNA NPs comprising RNA non-specific to a tumor in a subject provides therapeutic treatment for the tumor comparable to that provided by ML RNA NPs comprising RNA specific to the tumor, leading to increased percentage in animal survival.

### Example 11

This example demonstrates the ability of the nanoparticles of the present disclosure to elicit an immune response against SARS CoV-2.

Multilamellar RNA NPs are made as essentially described in Example 1. Naive mice are injected with the multilamellar RNA NPs. Mice are evaluated as essentially described in Example 5 to determine localization of the RNA NPs to the lungs. Blood obtained from the injected mice are assayed for neutralizing antibodies specific for the SARS CoV-2 S protein. Specifically, at 1, 6, 10, 14 and 21 days post administration of RNA NPs, blood samples are obtained from the mice. Samples are assayed for neutralizing antibodies against SARS-CoV-2 virus by following the technique described in Matsubara et al., PLoS One 8(7): e65281 (2013).

Binding antibodies also are assessed from human serum of vaccinated patients using a Coronavirus COVID-19 IgG ELISA Assay Kit (commercially available from Eagle Bioscience, Amherst NH, SKU: KT-1032). Neutralization is assessed using pseudoviruses. MLV-based pseudotypes (containing SARS-CoV-2 spike protein) are prepared as previously described in Millet and Whittaker, Bio Protoc. 2016 December 5; 6(23) (doi:10.21769/BioProtoc.2035). Briefly, HEK293T cells are co-transfected with a SARS-CoV-2 spike encoding-plasmid, an MLV Gag-Pol packaging construct, and a MLV transfer vector encoding a luciferase reporter. Cells are incubated for 5 hours at 37° C. with transfection medium. Cells are then washed with DMEM two times, and DMEM containing 10% FBS is added for 60 hours. The supernatants are harvested and filtered through 0.45-mm membranes, concentrated with a 30 kDa membrane for 10 min at 3,000 rpm, and then frozen at -80° C.

HEK293T cells stably transfected with ACE2 are then infected with prepared pseudovirus in the presence of human serum. ACE2 expressing 293T cells are seeded in 24-well plates at 2.5 × 10⁵ cells/well until confluent. Wells are then inoculated with 200 µl of pseudotyped virus solution, or maintained in non-infected control conditions (200 µl DMEM-C solution per well), with and without human serum from vaccinated patients. Cells are incubated at 37° C. 5% CO₂ for 1-2 hours before adding 300 µl DMEM-C. The cells are then incubated at 37° C. 5% CO₂ for 72 hours. Supernatants are aspirated. Both supernatants and cells are assessed for luciferase expression using a bioluminometer after addition of luciferin substrate. Successful neutralization is determined based on statistically significant decrease in luciferase expression with addition of human serum relative to control conditions.

### Example 12

This example describes a method of administering the nanoparticles of the present disclosure to a human subject.

Multilamellar RNA-NPs are made as described in Example 1 under GMP conditions. The NPs are administered to human subjects at 25 mcg, 100 mcg, or 250 mcg by injection. The following primary outcome measures are determined:
* Frequency of solicited local reactogenicity adverse events (AEs) [Time Frame: Through 7 days post-vaccination]
* Frequency of any medically-attended adverse events (MAAEs) [Time Frame: Day 1 to Day 394]
* Frequency of any new-onset chronic medical conditions (NOCMCs) [Time Frame: Day 1 to Day 394]
* Frequency of any serious adverse events (SAEs) [Time Frame: Day 1 to Day 394]
* Frequency of any unsolicited adverse events (AEs) [Time Frame: Through 28 days post-vaccination]
* Frequency of solicited systemic reactogenicity adverse events (AEs) [Time Frame: Through 7 days post-vaccination]
* Grade of any unsolicited adverse events (AEs) [Time Frame: Through 28 days post-vaccination]
* Grade of solicited local reactogenicity adverse events (AEs) [Time Frame: Through 7 days post-vaccination]
* Grade of solicited systemic reactogenicity adverse events (AEs) [Time Frame: Through 7 days post-vaccination]

This study is designed to assess the safety, reactogenicity and immunogenicity of the ML RNA NPs of the present disclosure.

### Example 13

This example describes a method of administering multilamellar RNA NPs of the present disclosure to induce in vivo an immune response against SARS-CoV-2.

To confirm the functional activity of the SARS-CoV-2 full-length spike mRNA, ML RNA NPs comprising mRNA encoding for SARS-CoV-2 full-length spike were administered to naïve C57BI/6 mice. Three vaccines were administered to the mice within a week.

About ten days after vaccine administration, peripheral blood mononuclear cells (PBMCs) were harvested from the mice and were assessed for memory recall response to ML RNA NPs by measuring ex vivo effector memory T cell expansion upon re-stimulation with overlapping SARS-CoV-2 spike peptides. Briefly, PBMCs were re-stimulated with 200 ng of overlapping peptide mix (PepMix™ SARS-CoV-2 (Spike Glycoprotein), JPT peptides) of 15-mers with 11-aa overlap from SARS-CoV-2 spike glycoprotein. Control PBMCs were unstimulated. PBMCs harvested from untreated mice (unvaccinated mice) served as another control. After culturing for 36 hours, PBMCs were harvested and stained for effector memory cells by staining for CD3+CD8+CD44+ subsets. The stained subsets of PBMCs were compared to those of PBMCs harvested from untreated mice (*p<0.05, **p<0.01, ***p<0.001, Mann-Whitney Test). The results are shown in FIGS. 21A and 21B.

As shown in FIG. 21A, mice that received the SARS-CoV-2 spike ML RNA NPs had more effector memory T cells after vaccination, with significant memory recall expansion, after in vitro re-stimulation with overlapping peptide mix for SARS-CoV-2 spike protein, compared to mice that did not receive the ML RNA NPs. (See dark circles on right in FIG. 21A.) The number of effective memory T cells increased upon re-stimulation with SARS-CoV-2 Spike protein compared to when not stimulated with the protein, suggesting the increase in T cells is a SARS-CoV-2-specific response. The supernatants of the co-culture assay wells also were sampled. After multiplex analysis, increased release of MIP-1-alpha (i.e., CCL3) was observed in re-stimulated cells, which is highly suggestive of Th1 mediated memory recall. See FIG. 21B. MIP-1-alpha is implicated in vaccination induced memory recall. In summary, mice receiving SARS-CoV-2 spike RNA-LPs had more effector memory T cells after vaccination, with significant memory recall expansion after in vitro re-stimulation with overlapping peptide mix for SARS-CoV-2 spike protein.

### Example 14

This example describes a method of administering the nanoparticles of the present disclosure to a human subject. In particular, this example describes a study characterizing the safety and immunologic activity of SARS-CoV-2 spike RNA-LP vaccines in adults (e.g., presence of SARS-CoV-2 specific neutralizing antibodies, SARS-CoV-2 specific T cells, and memory recall response over time in vaccinated patients).

This study represents a first in human Phase I study of SARS-CoV-2 spike/tumor RNA-LP vaccines for newly diagnosed adult MGMT unmethylated glioblastoma (GBM). The study involves a dose-escalation study using the BOIN design with an initial embedded accelerated titration design (ATD) to efficiently identify the maximally tolerated dose (MTD). One objective is to demonstrate the manufacturing feasibility and safety, and to determine the MTD of RNA-LP vaccines in adult patients with newly diagnosed MGMT unmethylated GBM. Patients with glioblastoma (GBM) are at increased risk of COVID-19 infection given significant lymphopenia seen with this disease and its treatment. A vaccine that both protects against COVID-19 while mediating therapeutic effects against the disease would provide a significant benefit.

The trial will consist of three parts: Surgery, Radiation, and Immunotherapy.

Surgery: Potentially eligible subjects will be enrolled on a screening consent for the sterile collection of tumor material in a manner suitable for RNA extraction, amplification, and loading of lipid particles (LPs). Tumor material will be sent to the University of Florida (UF) overnight. Following surgical resection with confirmatory pathologic diagnosis (at local institution), patients will be enrolled in the trial after informed consent has been obtained. Meanwhile investigators will generate tumor specific RNA-LP vaccines that will be shipped back to the enrolling site.

Radiation: Radiotherapy should begin within 4 weeks (+/- 14 days) of surgery or sooner based on institutional preference. Standard external beam RT will be administered concomitantly with TMZ (Stratum 1 only). Institutional practices for administration of external beam RT for subjects with GBM may be followed. Forty-two doses of temozolomide (TMZ) 75 mg/m²/day will be given continuously during radiation for up to 49 days to account for delays in radiation treatment. Delays or discontinuations of TMZ may be necessary due to ongoing clinical and laboratory assessment and tolerability of concomitant TMZ. Since eligibility is restricted to MGMT unmethylated primary adult type GBM, patients will not receive adjuvant cycles of temozolomide.

Standard external beam RT will be administered. Institutional practices for administration of external beam RT for subjects with adult MGMT unmethylated GBM may be followed. Otherwise the following guidelines should be used:

Target volume definition: (a) Gross Tumor Volume (GTV): The GTV includes all gross residual tumor and/or the tumor bed as defined by MR imaging and operative report. The GTV in many cases will involve a contracted or collapsed tumor bed. Tissue defects resulting from surgical approaches will not be included as part of the GTV when not previously involved by tumor, (b) Clinical Target Volume (CTV): The CTV is meant to treat subclinical microscopic disease and will be an anatomically constrained 5-10 mm margin on the GTV, with additional expansion as necessary to encompass areas of T2/FLAIR change suspicious for tumor involvement prior to surgery. The CTV is limited to the confines of the bony calvarium, falx and tentorium where applicable and extends up to but not beyond neuroanatomic structures through which tumor extension or invasion is certain not to have occurred. When the GTV approaches the boundary of an anatomic compartment, the CTV will extend up to and include the boundary.

Planning target volume 1 (PTV1): CTV +3 mm; Planning target volume 2 (PTV2): GTV + 3 mm.

Total dose: PTV1- 46 Gy at 2 Gy/fraction or 45-50.4 Gy at 1.8 Gy/fraction; PTV2- 59.4 - 60 Gy.

Dose per fraction: 1.8-2.0 Gy/fx daily, five days per week. PTV1 and PTV2 should be delivered in sequential phases over 30-33 fractions.

Immunotherapy: RNA-LP administration will begin within four weeks following radiation pending recovery of peripheral blood counts (i.e., ANC > 1500/฀L, platelets > 150/฀L), and after assessment of post-radiation MRI (for baseline). Higher thresholds are set since repeated RNA-NP administrations may elicit peripheral blood cytopenias. After radiation, patients will receive three RNA-LP vaccines every two weeks before beginning 12 cycles of adjuvant monthly RNA- LP vaccines for a total of 15 administrations.

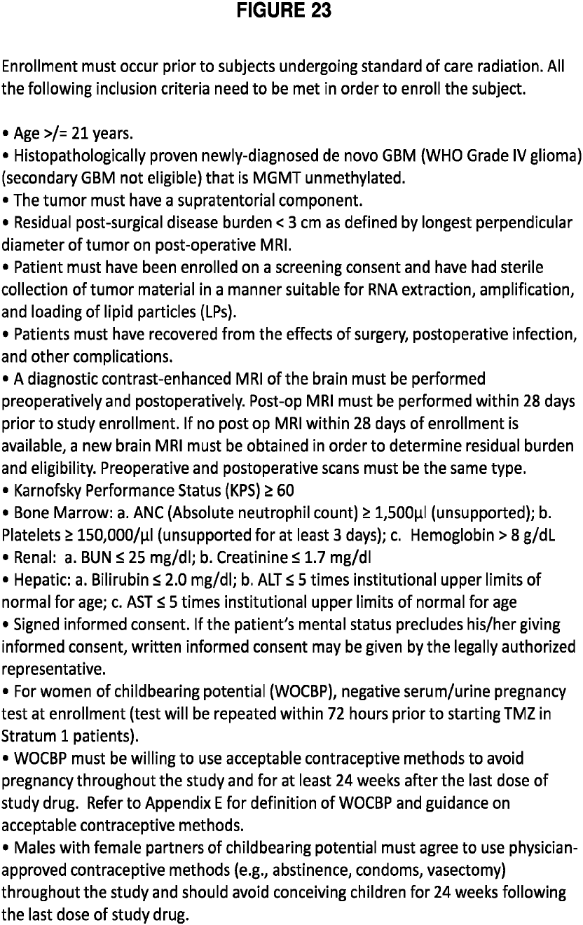

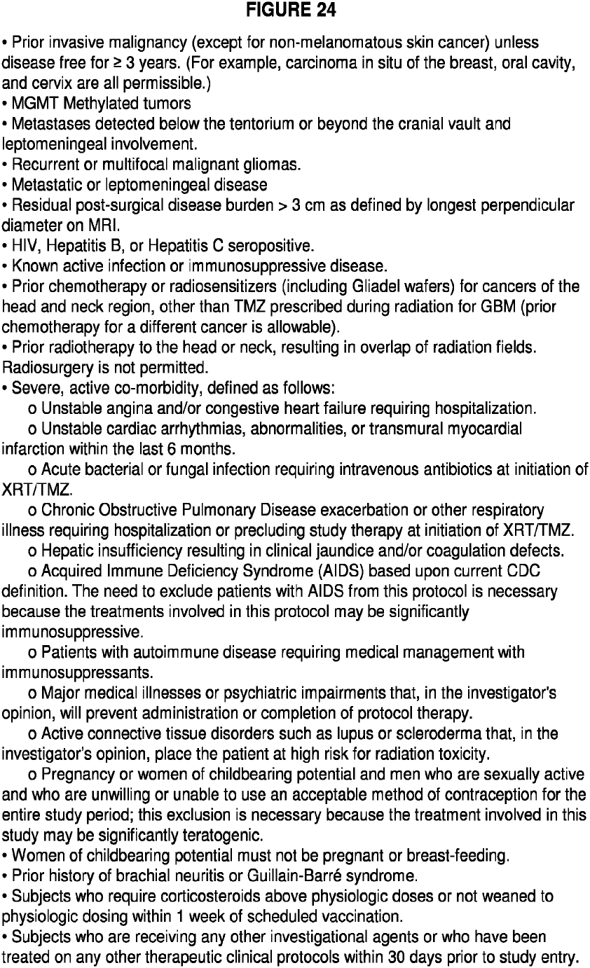

Subjects: A maximum of 28 adult patients will be enrolled in the dose-escalation study using the Bayesian optimal interval (BOIN) design with an initial embedded accelerated titration design (ATD). Inclusion and exclusion criteria are provided in FIGS. 23 and 24.

### Agent Administration

TMZ: Subjects will be administered (orally) temozolomide (TMZ), 75 mg/m²/day, during radiation (Stratum 1 only). Administration occurs on day 1 radiation therapy and proceeds for a maximum of 42 days. Generally, TMZ will be taken on an empty stomach (at least one hour before or two hours after food) at approximately the same time each day of radiation and weekends during radiation. Indeed, TMZ absorption is affected by food and, therefore, consistency of administration with respect to food is suggested. Preferably, administer at bedtime on an empty stomach (at least 1 hour before or 2 hours after food) to decrease nausea and vomiting and improve absorption. The whole dose, even if comprised of several capsule sizes, should be taken at one time at approximately the same time each day. Temozolomide dosing should be performed following institutional practice (e.g. +/- 5 to 10% of the calculated dose). It is recommended that antiemetics be given 30 minutes prior to each temozolomide dose. If emesis occurs within 20 minutes of taking a given dose, then the dose may be repeated once. If emesis occurs after 20 minutes, the dose should not be repeated. Oral suspension may be compounded if unable to swallow capsules. While receiving TMZ, subjects should receive PCP prophylaxis per institutional guidelines.

If radiotherapy has to be temporarily interrupted for technical or medical reasons unrelated to the temozolomide administration, then treatment with daily temozolomide should continue. If radiotherapy has to be permanently interrupted, then treatment with daily temozolomide should stop.

The 42 days of temozolomide should be given regardless of the end date of RT. If a dose of temozolomide is given and radiation therapy is NOT administered due to sedation or technical issues, the temozolomide doses should not be made up (i.e., no more than 42 doses of temozolomide should be given).

RNA-LP: The active immunotherapy components for RNA-LP vaccines are personalized tumor mRNA, SARS-CoV-2 spike mRNA, and DOTAP liposomes. These are referred to herein as RNA-LP. RNA-LP are administered intravenously (IV). A dose of RNA-LPs is administered every two weeks for an initial period of treatment lasting six weeks (three doses over six weeks, optionally on days 1, 15, and 29 of treatment), which is followed by 12 cycles of adjuvant monthly RNA-LP doses for a total of 15 administrations. A dose of RNA-LPs is administered IV at a rate of 0.04 mg/kg/hr, with appropriate flush volume.

Subjects will receive one of the doses of RNA-LP in Table 3 per administration.

TABLE 3

| | |
|---|---|
| Dose | Dose |
| Dose -4 | 0.000625 mg/kg of mRNA encapsulated in 0.009375 mg/kg LPs |
| Dose -3 | 0.00125 mg/kg mRNA encapsulated in 0.01875 mg/kg LPs |
| Dose -2 | 0.0025 mg/kg mRNA encapsulated in 0.0375 mg/kg LPs |
| Dose -1 | 0.005 mg/kg mRNA encapsulated in 0.075 mg/kg LPs |
| Dose 0 | 0.01 mg/kg mRNA encapsulated in 0.15 mg/kg LPs |
| Dose +1 | 0.02 mg/kg mRNA encapsulated in 0.3 mg/kg LPs |
| Dose +2 | 0.04 mg/kg mRNA encapsulated in 0.6 mg/kg LPs |
| Dose +3 | 0.08 mg/kg mRNA encapsulated in 1.2 mg/kg LPs |

At dose 0 (or at a lower dose at which a dose limiting toxicity (DLT) was observed), three adult patients will be treated with a total of three RNA-LP administrations, administered at an interval of every 2 weeks, after chemo-radiation. After the completion of the DLT period (two weeks after the completion of the first three RNA-LP administrations), if there are no safety concerns, enrollment will be opened for a pediatric cohort at the dose 0. If there are safety concerns (i.e., one or more DLTs observed), de-escalation to dose -1 will occur and, barring any safety concerns, that dose will be used as the starting dose for Stratum 2 (pediatric subjects). If toxicity assessments allow the pediatric stratum to open, the pediatric and adult phase I studies will follow separate dose escalations using the BOIN design and will have separate assessments of MTDs and toxicity.

While the initial DLT period will be evaluated two weeks after completion of the first three RNA-LP administration, before dose escalation (within the individual stratums), DLTs are monitored throughout the course of administrations.

Supportive care may include, but is not limited to, antibiotics, antiemetics, antidiarrheals, topical treatments, blood products, intravenous or oral fluids, electrolyte repletion, and will be used as clinically indicated. For signs and symptoms of pseudo-progression, treatment with bevacizumab per institutional guidelines is allowed to minimize swelling.

### Dose-Limiting Toxicity

A DLT will be defined as any immunotherapy-related (possible, probable or definite) per CTCAE 5.0 1) Grade 3 or greater non-neurologic, non-hematologic toxicity; 2) Grade 3 neurologic toxicity that does not improve to Grade II or better within 7 days or Grade 3-4 hematologic toxicity that does not improve to Grade 2 or better within 14 days; 3) Grade 3 autoimmune encephalomyelitis; or 4) Grade 4 neurologic toxicity. If neurologic toxicity returns to baseline prior to next administration, the next administration can be administered on schedule. Administration can be withheld for up to four weeks if neurologic symptoms have improved to grade 2 or below within 7 days (thus not a DLT) but have not returned to baseline by time of next scheduled vaccine. If the event cannot be reversed within 7 days of its onset (improving to Grade 2 or better), a DLT will be declared for that patient and no further administration will be performed (the DLT window is two weeks after the completion of the first three administrations). If the event is reversed, but toxicities defined above are again seen with subsequent vaccinations, patients will have further vaccinations withheld until discussion with DSMC.

Cerebral edema toxicity exception: CTCAE 5.0 criteria categorize cerebral edema as grade 3 (New onset; worsening from baseline), grade 4 (Life-threatening consequences; urgent intervention indicated) and grade 5 (death). Cerebral edema normally presents in patients with malignant gliomas as part of the disease process and can be exacerbated by standard of care chemotherapy and radiation. Furthermore, an effective anti-tumor immune response may involve inflammatory response and edema in infiltrative tumor cells. Therefore, cerebral edema toxicity, although ranked grade 3-4 by CTCAE 5.0 criteria, will be not be considered a DLT if patient is stable or improved clinically. If a cerebral edema is observed in a patient in clinical decline, the event will be considered a DLT if it is clearly attributable to the investigational drug and patient does not show improvement within 7 days of clinical management. Tumor progression will not be considered a DLT.

Grade 3 or greater toxicities associated with RNA-loaded DC-based immunotherapy have been rare. Adoptive T cell therapy using tumor-infiltrating lymphocytes and high-dose IL-2 in patients with melanoma and treatment with anti-CLTA-4 monoclonal antibody blockade have been the immunotherapy regimens most often associated with treatment related toxicities. To take the most conservative approach to assessing possible toxicities associated with this treatment, investigators should be vigilant for any similar toxicity associated with this therapy, in addition to autoimmune toxicity specific to the CNS. Possible immune-mediated disorders that have been observed in patients who have received immunotherapy in early phase trials have involved the skin (vitiligo and cutaneous leukocytoclastic vasculitis), the thyroid gland (autoimmune thyroiditis), the liver (autoimmune hepatitis) and the pituitary (hypophysitis). Abnormal lab results, which may be immune-mediated, include elevations of serum lipase and amylase and liver function tests. If a patient has an AE that is thought to be possibly related to autoimmune antibodies (e.g., thyroiditis, hepatitis, thrombocytopenia) the site PI will send a blood sample for appropriate autoimmune antibody testing per institutional standards. If specific autoantibodies are present, the serum sample taken at baseline will be tested for the presence of those autoantibodies.

### Assessments

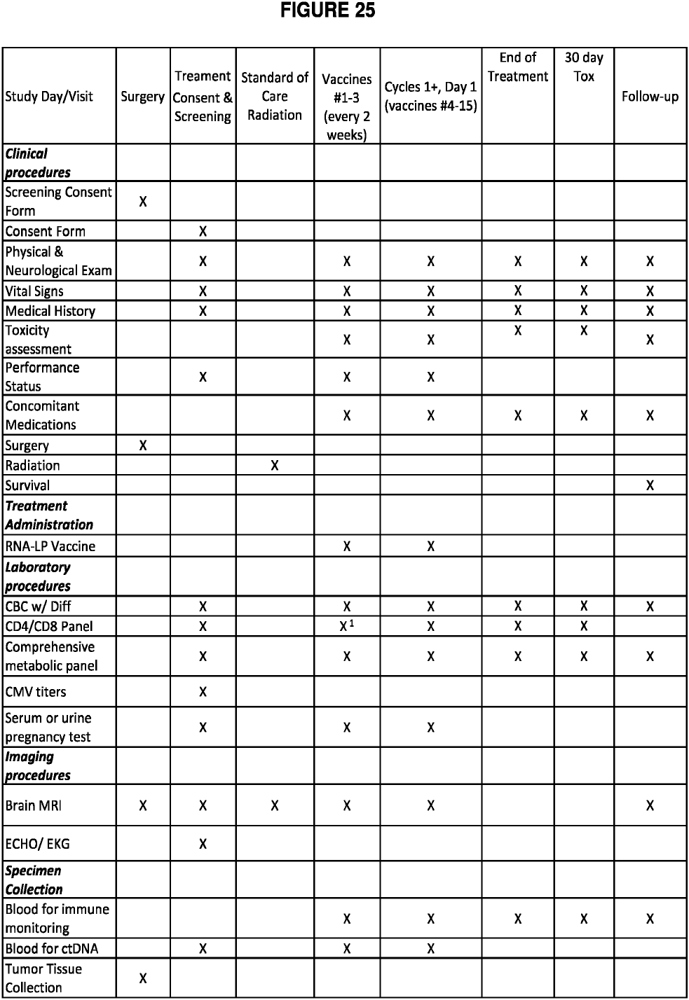

All assessments are allowed a +/- 3 day window unless otherwise noted. One cycle is 4 weeks (28 days). For additional details refer to the Observations and Procedures and Long Term Follow-up sections. A calendar of assessments is provided as FIG. 25, and a listing of assessments is provided below.

Standard of Care Resection/Biopsy: Screening informed consent ((Pre-surgery; within 28 days of standard of care resection/biopsy); Surgical resection/biopsy with tissue collection; MRI of brain (pre-op MRI must be performed within 28 days of surgery & a post-op MRI must be performed per institutional standard of care)

Screening (post-surgery) prior to Enrollment to Treatment (within 28 days of registration unless otherwise indicated): Confirmation of disease by pathology; Treatment Informed consent prior to radiation therapy; Physical & Neurological exam; Vital signs (including height & weight); Complete medical history including baseline symptoms, history of prior treatments and any residual toxicity relating to prior treatment; Performance status assessment; Brain MRI (does not need to be repeated if the post-op MRI was completed within 28 days of registration); Laboratory procedures (Serum pregnancy test for women of child bearing age, CBC with differential and platelet count, CD4/CD8 panel and ratio, PT/INR and PTT, Complete metabolic panel (CMP) including AST/ALT, BUN, Total bilirubin and creatinine, Infectious Disease testing including HIV, Hepatitis B, Hepatitis C, and CMV, Blood for circulating tumor DNA studies

Standard of Care Radiation (to begin 4 weeks (+/- 2 week after surgery) or sooner based on institutional preference): Administration of TMZ, Radiation per protocol or institutional guidelines for GBM

End of Radiation (+14 days): Post-radiation MRI

Administrations #1-3 (every 2 weeks +/- 3 days after hematologic recovery (ANC> 1500/฀L, platelets> 150/฀L): Administration of RNA-LP vaccine, Physical & Neurological exam, Vital signs (including height & weight), Performance Status, Adverse event assessment, Concomitant medications, Laboratory procedures (CBC with differential and platelet count, Complete metabolic panel (CMP) including AST/ALT, BUN, Total bilirubin and creatinine, CD4/CD8 panel and ratio (with administrations #1 and #3), Peripheral blood for immune-monitoring within 24 hours prior to vaccination, 2 and 6 hours post vaccination, Blood for circulating tumor DNA studies)

Cycles 1+, Day 1 (+/- 1 week), RNA-LP Administrations #4-15 (administration on Day 1 of each cycle following initial 3 administrations) (to begin 4 weeks after administration #3 once hematologic recovery is observed (ANC> 1500/฀L, platelets>150/DL): Administration of RNA-LP vaccine, Physical & Neurological exam, Vital signs (including height & weight), Adverse event assessment, Concomitant medications, Performance Status, Brain MRI (within 2 weeks of administration #4, 6, 8 10, 12, 14), Laboratory procedures (CBC with differential and platelet count, Complete metabolic panel (CMP) including AST/ALT, BUN, Total bilirubin and creatinine, CD4/CD8 panel and ratio (administration #5, 10 and 15), Peripheral blood for immune-monitoring within 24 hours prior to vaccination, 3 and 6 hours post vaccination, Blood for circulating tumor DNA studies)

End of treatment visit (+/-14 days if not previously assessed in this timeframe): Physical and neurological exam, Vital Signs, Adverse event assessment, Concomitant medications, Performance Status, Brain MRI

30 day toxicity check (+/- 7 days): Adverse event assessment- related AEs must continue to be followed until resolution or return to baseline, Concomitant medications

Long Term/Survival Follow-up Procedures: Patients will be followed until death due to any cause. As part of standard care for these patients, upon tumor progression, subjects may undergo stereotactic biopsy or resection. As this is not a research procedure, consent will be obtained separately. If tissue is obtained, it will be used to confirm tumor progression histologically and to assess immunologic cell infiltration and antigen expression profile in recurrent lesions.

Subjects will be followed every 3 months (+/- 14 days) for the first 12 months, and then every 6-12 months over the next 2 years until death due to any cause.

Year 1 (all procedures should be conducted every 3 months): Physical & Neurological exam, Vital signs (including height & weight), Medical history, MRI (+/- 14 days), Adverse event assessment, Concomitant medications; Optional- if feasible: CBC with differential and platelet count, CD4/CD8 panel and ratio, Complete metabolic panel including AST/ALT, BUN, Total bili and creatine, Immunotherapy labs and Peripheral blood for immune-monitoring

Year 2-3: MRI every 6-12 months, Adverse event assessment every 4-6 months, Concomitant medications every 4-6 months; Optional- if feasible: CBC with differential and platelet count, CD4/CD8 panel and ratio, Complete metabolic panel including AST/ALT, BUN, Total bili and creatine, Immunotherapy labs and Peripheral blood for immune-monitoring

Off-Treatment Criteria: For the purposes of this trial, “Off treatment” is defined as the day the last dose of investigational agent is administered. Treatment may continue for up to 14 months or until one of the following criteria applies: Disease progression, General or specific changes in the subject’s condition render the subject unacceptable for further treatment in the judgement of the investigator, Unacceptable adverse event(s), The subject, parent or legal guardian refuses further treatment on this protocol, Pregnancy

Off Study Criteria: Subjects will be considered Off Study for the following reasons: Subject determined to be ineligible, Subject, parent or legal guardian withdraws consent for continued participation, Subject death while on study, Completion of protocol specific follow up period

All subjects enrolled on the study, even if taken off study prior to treatment, will be included in the data analysis on an intent to treat basis. However, subjects will be replaced for safety assessments for the following reasons: (i) If RNA-LP vaccines are generated but do not meet release criteria or targeted dose then the patient may remain on study and receive the qualified product but will be replaced for the purposes of safety assessment. (ii) Subjects requiring an increase in corticosteroids, with the exception of nasal or inhaled steroid, such that at the time of first vaccination they require a dose above physiologic levels, may remain in the study but will be replaced for assessment of safety as increased steroid usage may mask risks of autoimmune toxicity. (iii) Subjects receiving less than 3 vaccines without toxicity will be replaced for safety assessments.

### Example 15

This Example describes additional studies characterizing the RNA-LPs of the disclosure.

An effective COVID-19 vaccine preferably elicits near-immediate protective responses (in the setting of a global pandemic) that overcomes both the SARS-CoV-2 cellular reservoir and its genomic heterogeneity (i.e., mutations). Current prophylactic strategies against COVID-19 utilize mRNA vaccines targeting small fragments of the SARS-CoV-2 genome, but these may not be strong enough or induce immunity quickly enough. These vaccines rely on generation of neutralizing antibodies, which require several boosts; however, viral reservoirs exist in host cells, which may necessitate new technologies that confer both T cell immunity to eliminate these reservoirs, and antibodies that neutralize active viral particles.

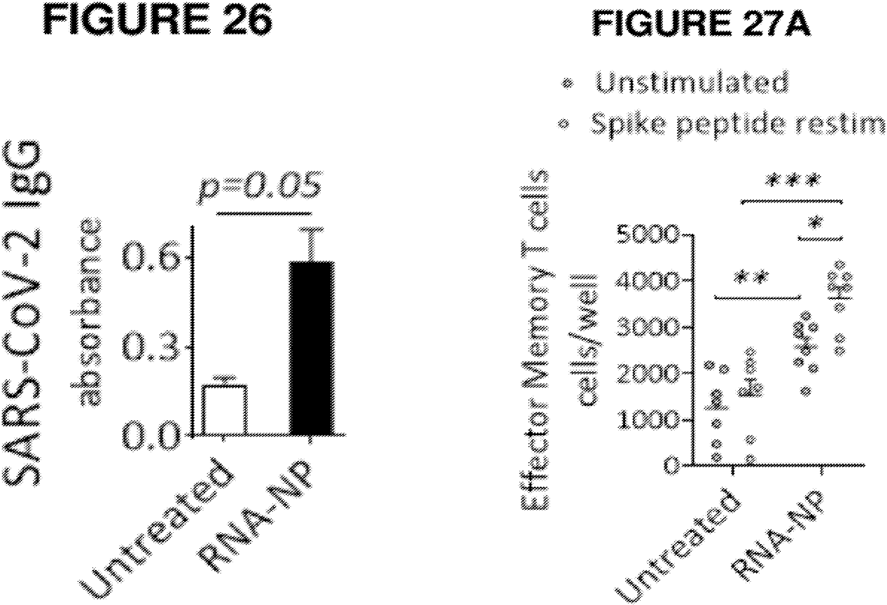

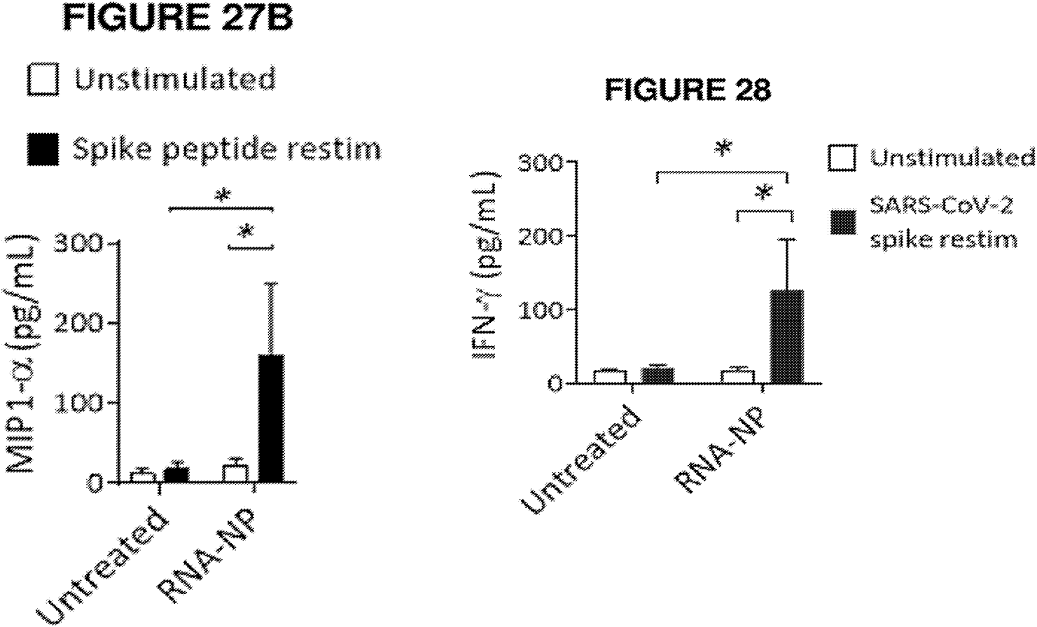

RNA-NP technology can address each of these concerns by, e.g., targeting full-length SARS-CoV-2 structural genes and overcoming mutational heterogeneity; inducing bidirectional adaptive immunity; and eliciting near immediate immune activation (within hours). Mice receiving SARS-CoV-2 full-length spike RNA-NPs had more SARS-CoV-2 specific antibodies (FIG. 26), and effector T cells after vaccination with significant memory recall expansion after in vitro re-stimulation with overlapping SARS-CoV-2 spike peptide mix (FIGS. 27A, 28B, and 28). RNA-NP activity is associated with IFN-y dependent innate immunity from plasmacytoid DCs (pDCs) in a non-TLR7 dependent manner.

Further studies were performed with human lung explants, demonstrating that the explants can be infected with coronaviruses including HCoV-OC43 and SARS-CoV-2. Lung tissue exposed to coronavirus before 48h culture shows evidence of dose-dependent viral transcription that can be attenuated with anti-viral drugs like remdesivir.

Additional information regarding studies suitable for characterizing RNA-LPs are described below.

### Neutralizing Ab Study

DNA sequence of SARS-CoV-2 spike, membrane, envelope, and nucleocapsid proteins is cloned into pGEM-4Z plasmid backbone embedded with a T7 promoter and polyAAA tail flanking regions of gene insert. Plasmids are transformed into E.coli and grown in presence of antibiotic (pGEM4z is ampicillin resistant) to select transformed E. coli, which is harvested for DNA extraction, linearization, and purification. DNA is then transcribed into mRNA, which is purified for loading into multi-lamellar RNA-NPs. Lipid-NPs composed of a DOTAP (Avanti) backbone is layered into multi-lamellar vesicles. Briefly, rotary vacuum evaporation is used to remove organic solvents, before resuspension in aqueous solution for rotational heating, bath sonication, extrusion and layering with SARS-CoV-2 specific mRNA in specific mass ratios of 1:15 (µg dosing, RNA to NP); 375 µg of lipid-NP formulation is complexed with 25 µg of SARS-CoV-2 specific mRNA, or control (non-specific) pp65 mRNA. C57BI/6 mice is i.v. vaccinated once weekly (x3); serum is harvested two weeks after the third vaccine for assessment of binding and neutralizing antibodies. Binding antibodies are assessed from serum of vaccinated mice using a customized coronavirus COVID-19 IgG ELISA kit. Neutralization is assessed using MLV-based pseudotypes (containing SARS-CoV-2 spike protein) as described below.

Pseudovirus System: Briefly, HEK293T cells are co-transfected with a SARS-CoV-2 spike, membrane, or envelope encoding-plasmid, a MLV Gag-Pol packaging construct, and a MLV transfer vector encoding a luciferase reporter. Cells are incubated for 5 h at 37° C. with transfection medium. Cells are then washed with DMEM two times and then DMEM containing 10% FBS will be added for 60 h. The supernatants are harvested and filtered through 0.45-mm membranes, concentrated with a 30 kDa membrane for 10 min at 3,000 rpm and then frozen at -80° C. HEK293T cells stably transfected with ACE2 (creative-biogene.com) are infected with prepared pseudovirus in the presence of murine serum. ACE2 expressing 293T cells are seeded to 24-well plates at 2.5 × 10⁵ cells/well until confluent and inoculate wells with 200 µl of pseudotyped virus solution or non-infected control conditions (200 µl DMEM-C solution per well) with and without serum from vaccinated animals. Incubation occurs at 37° C. 5% CO₂ for 1-2 h before adding 300 µl DMEM-C for 72 h incubation. Supernatants are aspirated and both supernatants and cells are assessed/quantified for luciferase expression using a bioluminometer after addition of luciferin substrate. Successful neutralization is determined based on statistically significant decrease in luciferase expression following addition of mouse sera relative to control conditions. Results may be confirmed in 3D culture system using pulmonary explants co-cultured with serum from animals vaccinated with RNA-NPs.

### Responsive Memory T Cell Analysis

SARS-CoV-2 specific RNA-NPs encoding for spike, membrane protein, envelope and nucleocapsid genes are given separately to C57BI/6 mice and humanized HLA-A2 transgenic mice. RNA-NPs are administered by intravenous injection on day 0, 7, 14, followed by weekly assessment of blood/serum. Serum is analyzed for detection of Th1 cytokines (i.e., CCL3, TNF-α, and IFN-y) by bead array (BD), and T cells will be assessed for presence of activation markers (i.e., CD107a, CD69, CD154, PD-1). Antigen specificity against spike and nucleocapsid structural proteins is determined by HLA*2 MHC- Class I pentamer staining (Pro-Immune), and ex vivo memory recall response. Recall responsive T cells are identified based on ex vivo restimulation assays of PBMCs from vaccinated animals with bone marrow derived dendritic cells (from naïve mice) pulsed with mRNA encoding for SARS-CoV-2 specific structural proteins versus dendritic cells pulsed with control mRNA encoding for pp65 (human CMV matrix protein). These DCs are co-cultured with magnetically separated CD4 and CD8 T cells from vaccinated mice, and assessment of T cells is performed for proliferation, phenotype (effector versus central memory by differential staining for CD44 and CD62L), function and cytotoxicity. Ex vivo co-cultures are performed in triplicate, for 48 h in a 96 well plate (4×10⁴ DCs will be ex vivo co-cultured with 4×10⁵ T cells per well). T cells are labeled with CFSE (Celltrace) for assessment of proliferation by flow cytometry, and re-stimulated in culture before supernatants and cells are harvested. To determine cytotoxicity following co-culture, T cells are incubated in the presence of ACE2 expressing 293T cells containing a luciferase bioreporter (infected with pseudovirus as described above) or control cells cultured in a 10:1 effector/target ratio × 48 h and assessed for bioluminescence. Bioluminescence from each co-culture, as a surrogate for living cells, is quantitatively measured by IVIS imaging.

### Study of Innate Signaling Mechanisms

Activity from RNA-NPs is dependent on IFN-y (type I interferon) from plasmacytoid DCs (pDCs). Interestingly, the innate immunomodulating effects of RNA-NPs do not appear to be driven by toll-like receptor (TLR) stimulation, as efficacy is intact in TLR7 KO mice. This study explores whether SARS-CoV-2 specific RNA-NPs elicit bidirectional adaptive responses in an IFN-y dependent manner, and identifies cell types and signaling pathways involved. RNA-NPs may reset IFNAR1 signaling through non-TLR dependent intracellular PRRs from pDCs. RIG-I, MDA-5 or STING may be involved in IFN-γ signaling.

SARS-Co-V-2 specific RNA-NPs, pp65 RNA-NPs or NPs alone are administered to mice as described above. Vaccines are administered once weekly (x3). Mice are vaccinated 3 times at 7-day intervals. Blood is collected and assessed for DC subpopulations. pDCs are stained for CD11c, B220 and Gr-1 (ebioscience); distinct pDC subsets are identified by differential staining for CCR9, SCA1, and Ly49q. Resident/migratory classical DCs (cDCs) are identified by CD11c+CD103+MHCII+cells and CD11c+CD11b+MHCII+cells; myeloid DCs (mDCs) are identified by CD11c+CD14+MHCII+cells. Activation state is assessed based on expression of cytokines (i.e., IFN-I/II, IL-12) and co-stimulatory molecules (i.e., CD40, CD80, CD86). Analyses are conducted by multi-parameter flow cytometry (LSR, BD Biosciences). Innate pathways (i.e., RIG-I, MDA-5 and STING) of interest are explored in these cell subsets by PCR. Results may be correlated with SARS-CoV-2 neutralization and adaptive recall as described above. To confirm if results are dependent on IFN-I (i.e., IFN-α/IFN-β) from intracellular PRRs, wild-type and knock-out (KO) mice are vaccinated for pathways of interest (i.e., IFNAR1, RIG-I, MDA-5 and STING, all available through Jackson labs). IFN-I (i.e. IFN-α/IFN-β) levels are measured from serum by ELISA 24 h post-vaccine and compared with results with anti-SARS-CoV-2 immunogenicity testing as described above.

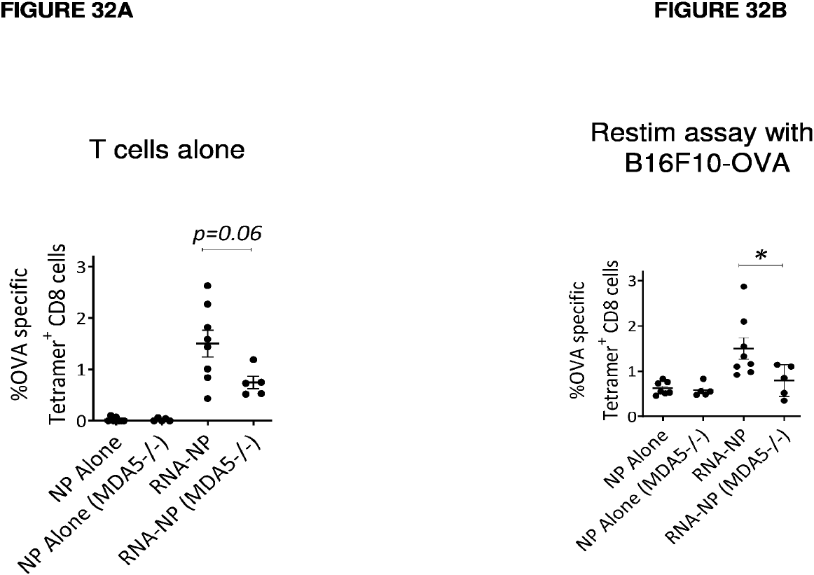

Immunogenicity of multi-lamellar RNA-NPs appears to be dependent on intracellular pathogen recognition receptors such as MDA-5, in contrast to existing mRNA nanolipid platforms. FIGS. 32A and 32B.

### Examining Relationship Between Gene Expression and Immunogenicity

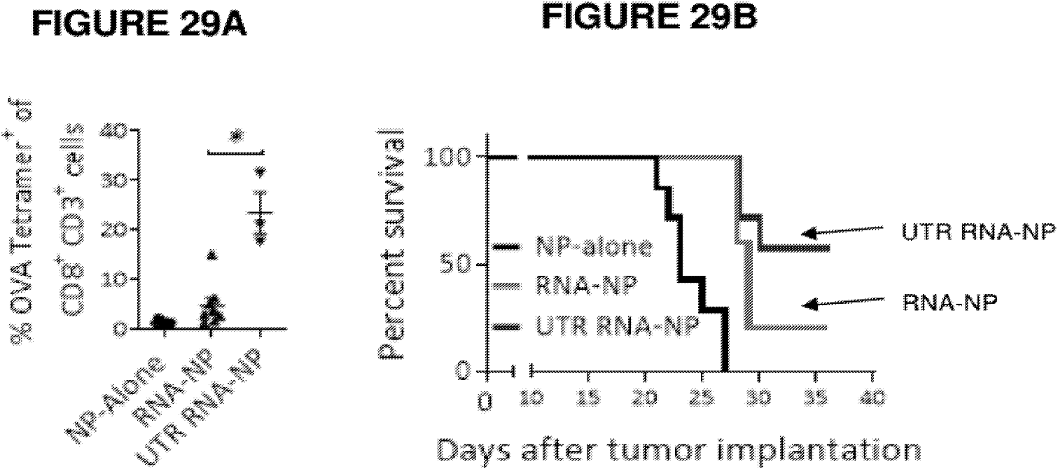

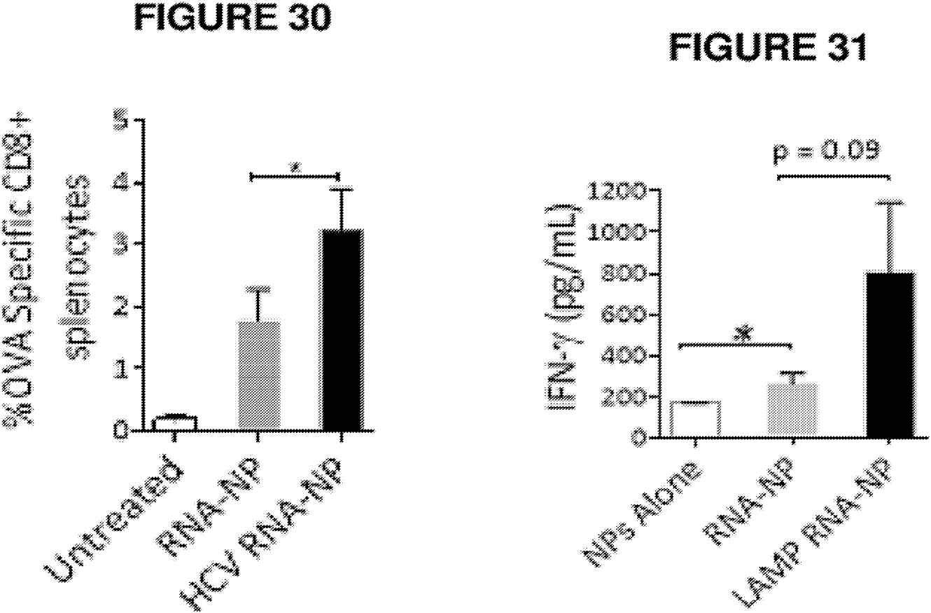

The 5′ internal ribosomal entry site (IRES) of hepatitis C virus (HCV) leads to cap-independent translation and very rapid and extensive translation of the RNA genome while its 3′ polyUUU tail is recognized by PRRs including RIG-I and MDA5 pathways lead to intense triggering of the innate immune system and production of type I interferons. Addition of alpha-globin UTRs (FIGS. 29A and 29B) and the 5′ and 3′ UTRs from HCV (FIG. 30) enhances activity of RNA-NPs. This study explores whether HCV’s 5′ UTR alone or in conjunction with the 3′ UTR enhances RNA stability/immunogenicity of SARS-CoV-2 RNA-NP constructs.

5′ alpha globin UTRs, HCV’s 5′ IRES and 3′ HCV polyUUU tail are incorporated through restriction digest and ligation into pGEM4z plasmid vector; these sequences were cloned into DNA plasmids for luciferase which will be used to determine if modifications enhance stable gene expression. RNA templates are made using PCR products isolated via gel electrophoresis and purified using the QIAquick Gel Extraction Kit. The resulting PCR fragments are digested with the restriction enzymes Hindlll and EcoRI and cloned into the Hindll and EcoRI sites of the plasmid which has a T7 promoter and 64T nucleotides that allow for the production of in vitro transcribed RNA with a polyA tail of 64 residues. The plasmids are linearized with Spel, in vitro transcribed (IVT) using mMessage mMachine T7 (Ambion), purified with RNeasy kit (Qiagen), quantified by spectrophotometry, and analyzed by an Agilent Bioanalyzer to confirm quality and synthesis of full-length mRNA.

Expression of luciferase based constructs with immunogenicity testing for SARS-CoV-2 RNA-NP constructs is compared with unmodified SARS-CoV-2 RNA-NP constructs using the assays described above.

### Examination of LAMP-Conjugated RNA-NPs

LAMP-1 and DC-LAMP targeting results in increased T cell responses, most notably increased CD4+ T cell ‘help’ through the channeling of antigens into the MHC class II presentation pathway. This results in a broader repertoire of CD4+ responses, culminating in enhanced humoral immunity and polyfunctional T cell responses. Since CD4+ immune responses are integral for induction and maintenance of active immunity against viral infections, LAMP conjugated antigens are expected to potentiate bidirectional response against SARS-CoV-2. LAMP incorporation into RNA-NPs has potential to significantly increase memory recall response (FIG. 31).

LAMP-1 and DC-LAMP constructs are made by inserting the SARS-CoV-2 specific genes between the luminal domain and the transmembrane domain and cytoplasmic tail of LAMP before cloning into pGEM-4z vector. The immunologic effects of LAMP modified is compared to unmodified RNA-NPs in: 1) control RNA-NPs; 2) RNA-NPs incorporating LAMP-1 targeted SARS-CoV-2antigens; 3) RNA-NPs incorporating DC-LAMP targeted SARS-CoV-2 antigens; and 4) RNA-NPs incorporating the combination of DC-LAMP and LAMP-1 targeted antigens (equimolar ratio) using the assays described above.

### Bidirectional Adaptive Immunity

SARS-CoV-2 structural mRNAs are compared with unmodified targets. Unmodified SARS-CoV-2 RNA-NPs are compared with UTR modified RNA-NPs, LAMP modified RNA-nanoparticle vaccine, and UTR + LAMP modified RNA-NPs. The effects of smaller and less frequent dosing is examined with respect to anti-SARS-CoV-2 adaptive responses. We will compare effects of decreasing doses (i.e., 10 µg, 5 µg 2.5 µg, 1 µg) versus standard 25 µg dose given as a single vaccine or given as multiple weekly vaccines (x3). Immunogenicity is examined as described above, and results may be corroborated in HLA-A2 and ACE2 transgenic animals. UTR and LAMP modified vaccines targeting multiple SARS-CoV-2 structural proteins may elicit more effective protection as a single administration at low doses.

### Formulation Study

RNA-NPs are re-suspended in chloroform before being evaporated off until a thin lipid layer remains. For in vivo preparations, SARS-CoV-2 specific mRNA is added to DOTAP NPs at a multilamellar ratio of 1:15 mg. This mixture is kept at room temperature for 15-20 minutes to allow RNA-NP complexes to form. After multi-lamellar complexation, contents are mixed with tertiary butanol, vortexed and frozen at -80° C. overnight before being lyophilized (Labconco) and stored at -20° C. Validation of lyophilized formulation is assessed based on anti-SARS-CoV-2 mediated humoral and cellular responses compared with freshly produced RNA-NPs. Particles are validated by zeta-potential measurements and size measurements.

### Additional in Vivo Studies

For a large animal study in felines, RNA-NPs are injected i.v. to define safety and toxicity. Starting (low) dose of RNA-NPs involves: RNA (0.05 mg/kg) encapsulated with 0.75 mg/kg of DOTAP nanoliposome. These are compared to medium doses (100% increase) of 0.1 mg/kg of RNA encapsulated with 1.5 mg/kg of DOTAP nanoliposome, which is the feline equivalent dose (based on body surface area) of the dosing in mice (1 mg/kg). High dose administrations (100% increase) will involve 0.2 mg/kg of RNA encapsulated with 3 mg/kg of DOTAP nanoliposome. Feasibility of SARS-CoV-2 specific mRNA production is determined based on RNA quality, concentration and integrity by gel electrophoresis, nanodrop spectrophotometry and bioanalysis. Animals randomized to receive multiple dosing receive three RNA-NPs one week apart. Peripheral labs are monitored from felines at enrollment, immediately before each RNA-NP, and during 1-month post-treatment follow-ups. Complete blood counts (with differentials), comprehensive chemistries, lipid panels and renal/liver function tests are monitored. Serum cytokines will be obtained post-infusion to assess for symptoms of cytokine release syndrome (i.e., IL-1, IL-6, TNF-a).Increasing doses of i.v. RNA-NPs are injected to define dose limiting toxicities (DLTs), such as grade 3-4 adverse events and maximal tolerated dose (MTD) in a standard 3+3 study design. There are three groups (three felines/group). If DLT attributable to RNA-NPs is experienced in 1 of 3 felines, an additional 3 felines will be enrolled at the same dose. If none of 3 felines or 1 of 6 felines experiences DLT, dose will be escalated in subsequently accrued felines. If ≥2 felines experience DLT attributable to the RNA-NPs, the accrual to that group will be halted and the immediate lower dose level will be considered the MTD. Based on immunologic response across subjects from 3+3 study across each arm, the lowest effective dose of SARS-CoV-2 RNA-NPs is selected to advance into an expansion cohort (up to 20 subjects per arm).

To confirm that SARS-CoV-2 specific RNA-NPs mediate increases in SARS-CoV-2 specific neutralizing antibodies and recall memory T cells that persist in large animals, immediately preceding each treatment and during post-treatment follow-up (30 days after last vaccine, at 6 mo and 12 mo), 10 mLs of peripheral feline blood is drawn into vacutainer tubes. PBMCs are separated by density gradient centrifugation via ficoll. DCs and T cells are assessed from the peripheral blood for determination of activation markers (i.e., CD80, CD86, and MHCII on CD11c+ cells and CD44 on CD4 and CD8+ cells). PBMCs electroporated with SARS-CoV-2-specific mRNA (or control pp65 mRNA) are co-cultured with CD3 selected T cells before assessment of IFN-y and MIP-1-α (i.e., CCL3) from supernatants by ELISA. T cell cytotoxicity against SARS-CoV-2 is assessed. Serum from animals is drawn one, six and 12 months after the last vaccine for assessment of neutralization in 3D pulmonary explants. Neutralization against SARS-CoV-2 and related coronaviruses such as HcoV-OC43 in 3D is assessed in pulmonary explants.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure. Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A nanoparticle comprising a positively-charged surface and an interior comprising (i) a core and (ii) at least two nucleic acid layers, wherein each nucleic acid layer is positioned between a cationic lipid bilayer, wherein the nanoparticle comprises RNA molecules encoding a SARS-CoV-2 protein.

2. (canceled)

3. The nanoparticle of claim 1, comprising at least four nucleic acid layers, each of which is positioned between a cationic lipid bilayer.

4. (canceled)

5. The nanoparticle of claim 1, wherein the outermost layer of the nanoparticle comprises a cationic lipid bilayer.

6. (canceled)

7. The nanoparticle of claim 1, wherein the core comprises a cationic lipid bilayer.

8. The nanoparticle of claim 1, wherein the core comprises less than about 0.5 wt% nucleic acid.

9. The nanoparticle of claim 1, wherein the diameter of the nanoparticle is about 50 nm to about 500 nm in diameter.

10. The nanoparticle of claim 1, comprising a zeta potential of about 40 mV to about 60 mV.

11. The nanoparticle of claim 10, comprising a zeta potential of about 50 mV.

12. The nanoparticle of claim 1, comprising nucleic acid molecules and cationic lipid at a ratio of about 1 to about 5 to about 1 to about 20.

13. The nanoparticle of claim 1, wherein the cationic lipid is DOTAP or DOTMA.

14. (canceled)

15. (canceled)

16. The nanoparticle of claim 1, wherein the nucleic acids are mRNAs encoding a SARS-CoV-2 Spike (S) protein or a fragment thereof.

17. The nanoparticle of claim 16, wherein the S protein comprises the amino acid sequence set forth in Figure 20.

18. The nanoparticle of claim 1, wherein the nucleic acids are mRNAs encoding a SARS-CoV-2 membrane protein, envelope protein, or a nucleocapsid protein.

19. The nanoparticle of claim 1, wherein the liposomes are prepared by mixing the nucleic acid molecules and the cationic lipid at a RNA: cationic lipid ratio of about 1 to about 5 to about 1 to about 20, optionally, about 1 to about 15.

20. A method of making a nanoparticle comprising a positively-charged surface and an interior comprising (i) a core and (ii) at least two nucleic acid layers, wherein each nucleic acid layer is positioned between a cationic lipid bilayer, wherein the nanoparticle comprises RNA molecules encoding a SARS-CoV-2 protein, said method comprising:
(A) mixing nucleic acid molecules encoding a SARS-CoV-2 protein and liposomes at a nucleic acid: liposome ratio of about 1 to about 5 to about 1 to about 20, optionally, about 1 to about 15, to obtain a nucleic acid-coated liposomes, wherein the liposomes are made by a process of making liposomes comprising drying a lipid mixture comprising a cationic lipid and an organic solvent by evaporating the organic solvent under a vacuum; and
(B) mixing the nucleic acid-coated liposomes with a surplus amount of liposomes.

21-31. (canceled)

32. A cell comprising a nanoparticle as described in claim 1.

33. (canceled)

34. (canceled)

35. A pharmaceutical composition comprising a plurality of nanoparticles according claim 1 and a pharmaceutically acceptable carrier, diluent, or excipient.

36. (canceled)

37. A method of inducing an immune response against a SARS-CoV-2 virus in a subject, the method comprising administering to the subject the pharmaceutical composition of claim 35.

38-44. (canceled)

45. The method of claim 37, wherein a single dose of the composition comprises about 0.00050 mg/kg to about 1.5 mg/kg of nucleic acid.

46-48. (canceled)

49. The method of claim 37, comprising administering to the subject multiple doses of pharmaceutical composition over a treatment period of about 18 months.

50. The method of claim 49, comprising administering (a) an initial set of doses of the pharmaceutical composition, each administration separated by two weeks, followed by (b) a subsequent set of doses of pharmaceutical composition, each administered once per month.

51. The method of claim 50, wherein the initial set of three doses are administered over an initial treatment period of about four weeks, and the subsequent set of doses are administered over a subsequent treatment period of about 12 months.
